(19) United States
(12) Patent Application Publication
(10) Pub. No.: US 2026/0008996 A1
(43) Pub. Date: Jan. 8, 2026

(54) Compositions and Methods for Modification of Cells

(71) Applicant: The Regents of the University of California, Oakland, CA (US)

(72) Inventors: Matthew B. Francis, Berkeley, CA (US); Jonathan Charles Maza, Berkeley, CA (US); Derek Garcia Almedina, Berkeley, CA (US)

(21) Appl. No.: 18/859,936

(22) PCT Filed: May 10, 2023
(86) PCT No.: PCT/US2023/066808

§ 371 Date: Oct. 24, 2024

Related U.S. Application Data
(60) Provisional application No. 63/340,691, filed on May 11, 2022.

(51) Int. Cl. C12N 5/00; C07K 16/44; C12N 9/02

(52) U.S. Cl. CPC C12N 5/0006; C07K 16/44; C12N 9/0071; C12Y 114/18001; C07K 2317/569; C07K 2319/60

(57) ABSTRACT

The present disclosure provides a method for chemoselective modification of a cell surface molecule on a target cell. A subject method includes contacting a target cell comprising cell surface molecule comprising a thiol, an amine, or an imidazole with a biomolecule comprising a reactive moiety, wherein the reactive moiety is generated by reaction of a biomolecule (e.g., an antibody) comprising a phenol moiety or a catechol with an enzyme capable of oxidizing the phenol or the catechol moiety. The contacting is carried out under conditions sufficient for conjugation of the cell surface molecule to the biomolecule, thereby producing a modified cell. The present disclosure provides kits for carrying out a subject method. The present disclosure also provides modified cells and methods for using same.

### CROSS-REFERENCE

This application claims the benefit of U.S. Provisional Patent Application No. 63/340,691, filed May 11, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “BERK-464WO_SEQ_LIST” created on Apr. 27, 2023 and having a size of 204,705 bytes. The contents of the Sequence Listing XML are incorporated by reference herein in their entirety.

### INTRODUCTION

Coupling biomolecules to target molecules on the surface of living cells, to generate conjugates, while preserving the function of the biomolecule and the target molecule, and while maintaining viability of the cell, has long been a goal of chemical biology and biopharmaceutical research. The ability to manipulate the cell surface has a broad range of applications in basic and applied science. For example, the introduction of cancer-antigen binding proteins to the surface of immune cells has led to the development of cell-based immunotherapies against a variety of cancers. While genetic engineering has been a key technology in many of these applications, new chemistries are emerging for the direct attachment of complex molecules to cell surfaces. Chemistries that modify cells must perform under a narrow set of conditions in order to maintain cell viability. They must proceed in buffered aqueous media at the optimal physiological pH—typically pH 7.4—and within a temperature range of 4-37° C. Furthermore, these reactions must have sufficiently rapid kinetics to achieve high conversion even when confronted with the limits of surface diffusion characteristics. Due to these requirements, few chemistries exist that can attach molecules and proteins to live cells.

There is a need for improved methods of attaching proteins to living cells.

### SUMMARY

The present disclosure provides a method for chemoselective modification of a cell surface molecule on a target cell. A subject method includes contacting a target cell comprising cell surface molecule comprising a thiol, an amine, or an imidazole with a biomolecule comprising a reactive moiety, wherein the reactive moiety is generated by reaction of a biomolecule (e.g., an antibody) comprising a phenol moiety or a catechol with an enzyme capable of oxidizing the phenol or the catechol moiety. The contacting is carried out under conditions sufficient for conjugation of the cell surface molecule to the biomolecule, thereby producing a modified cell. The present disclosure provides kits for carrying out a subject method. The present disclosure also provides modified cells and methods for using same.

### BRIEF DESCRIPTION OF THE DRAWINGS

The invention is best understood from the following detailed description when read in conjunction with the accompanying figures. It is emphasized that, according to common practice, the various features of the figures are not to-scale. On the contrary, the dimensions of the various features are arbitrarily expanded or reduced for clarity. Included in the drawings are the following figures. It is understood that the figures, described below, are for illustration purposes only. The figures are not intended to limit the scope of the present teachings in any way.

FIG. 1A-1C illustrate a general strategy for modifying cell surfaces with nanobodies.

FIG. 2A-2D depict modification of natural killer (NK) cell surfaces with nanobodies.

FIG. 3A-3C depict decoration of NK cells for nanobody-directed cell-cell interactions.

FIG. 4A-4C depict targeted cell killing elicited by tyrosinase-synthesized nanobody-NK cell conjugates.

FIG. 5A-5B depict the results of nanobody oxidation tests.

FIG. 6 depicts evaluation of NK-92MI cell viability following modification with nanobodies and abTYR.

FIG. 7 depicts synthesis of FITC-labeled nanobody.

FIG. 8A-8C depict synthesis of nanobody-Jurkat cell conjugates.

FIGS. 9 and 10 provide mushroom tyrosinase amino acid sequences (SEQ ID NOs:1-2, respectively).

FIGS. 11A-11Z and 11AA-11VV provide Bacillus megaterium tyrosinase amino acid sequences (SEQ ID NOs:3-50, respectively).

### DEFINITIONS

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a thiol group” includes a plurality of such thiol groups and reference to “the thiol group” includes reference to one or more thiol groups and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, the term “affinity tag” refers to a member of a specific binding pair, i.e. two molecules where one of the molecules through chemical or physical means specifically binds to the other molecule. The complementary member of the affinity tag may be immobilized (e.g., to a chromatography support, a bead or a planar surface) to produce an affinity chromatography support that specifically binds the affinity tag. Tagging a compound of interest with an affinity tag allows the compound to be separated from a mixture of untagged compounds by affinity, e.g., using affinity chromatography. Examples of specific binding pairs include biotin and streptavidin (or avidin), and antigen and antibody, although binding pairs, e.g., nucleic acid hybrids, polyhistidine and nickel, and azido and alkynyl (e.g., cyclooctynyl) or phosphino groups are also envisioned. The specific binding pairs may include analogs, derivatives and fragments of the original specific binding members.

As used herein, the term “biotin moiety” refers to an affinity tag that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10⁻⁸M. A biotin moiety may also include a linker, e.g., -LC-biotin, -LC-LC-Biotin, -SLC-Biotin or -PEGₙ¹-Biotin where n¹ is 3-12.

By “linking” or “linker” as in “linking group,” “linker moiety,” etc., is meant a linking moiety that connects two groups via covalent bonds. The linker may be linear, branched, cyclic or a single atom. Examples of such linking groups include alkyl, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), epidithio (—S—S—), carbonyldioxy (—O—CO—O—), alkyldioxy (—O—(CH₂)n-O—), epoxyimino (—O—NH—), epimino (—NH—), carbonyl (—CO—), etc. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. Any convenient orientation and/or connections of the linkers to the linked groups may be used.

“Alkyl” refers to monovalent saturated aliphatic hydrocarbyl groups having from 1 to 10 carbon atoms, e.g., from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ²— (where n² is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO— heteroaryl, —SO₂-alkyl, —SO₂-substituted alkyl, —SO₂-aryl, —SO₂-heteroaryl and trihalomethyl.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸¹R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O-M⁺, —OSO₂OR⁷⁰, —P(O)(O⁻)₂(M⁺)₂, —P(O)(OR⁷⁰)O M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷¹ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃-M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO2R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include C and all isotopes of carbon (such as ³C).

As used herein, the term “cleavable linker” or “cleavably linked” refers to a linker or a linkage that is selectively breakable using a stimulus (e.g., a physical, chemical or enzymatic stimulus) that leaves the moieties to which the linkages joins intact. Several cleavable linkages have been described in the literature (e.g., Brown (1997) Contemporary Organic Synthesis 4(3); 216-237). And Guillier et al (Chem. Rev. 2000 1000:2091-2157). A disulfide bond (which can be broken by DDT) and a photo-cleavable linker are examples of cleavable linkages.

The term “fluorophore” refers to any molecular entity that is capable of absorbing energy of a first wavelength and re-emit energy at a different second wavelength. In certain embodiments, the subject biomolecule includes a fluorophore attached to one end of the biomolecule or at a central position. In some embodiments, the fluorophore may be attached to one end of the biomolecule. The fluorophore attached to the biomolecule need not be a single molecule, but may include multiple molecules.

The fluorophore may be synthetic or biological in nature, as known to those of skill in the art. More generally, any fluorophore can be used that is stable under coupling conditions and that can be sufficiently suppressed when in close proximity to the quencher such that a significant change in the intensity of fluorescence of the fluorophore is detectable in response to target specifically binding the probe. Examples of suitable fluorophores include, but are not limited to Oregon Green 488 dye, rhodamine and rhodamine derivatives, fluorescein isothiocyanate, fluorescein, 6-carboxyfluorescein (6-FAM), coumarin and coumarin derivatives, cyanine and cyanine derivatives, Alexa Fluors, DyLight Fluors, and the like.

In certain embodiments, the biomolecule includes a metal-chelating agent. A “chelate” as used herein in reference to a complex between a metal and a chelating ligand, refers to a combination of a metallic ion bonded to one or more ligands to form a heterocyclic ring structure. Chelate formation through neutralization of the positive charge(s) of the metal ion may be through the formation of ionic, covalent or coordinate covalent bonding. In certain embodiments, the metal-chelating agent is includes, but are not limited to, 1,4,7,10-Tetraazacyclododecane-1,4,7,10-tetraacetic acid (also referred to as, DOTA, or tetraxetan).

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term “polypeptide” encompasses a chemically modified polypeptide. For example, the term “polypeptide” can include a monovalent radical of i) a chemical structure of a polypeptide or peptide or ii) a chemical structure of a polypeptide or peptide substituted with a chemical functional group. The term “fusion protein” or grammatical equivalents thereof is meant to include a protein composed of a plurality of polypeptide components, that while typically unjoined in their native state, typically are joined by their respective amino and carboxyl termini through a peptide linkage to form a single continuous polypeptide. Fusion proteins may be a combination of two, three or even four or more different proteins. Polypeptides include structural polypeptides and polypeptide enzymes.

As used herein, the term “a target protein” refers to all members of the target family, and fragments and enantiomers thereof, and protein mimics thereof. The target proteins of interest that are described herein are intended to include all members of the target family, and fragments and enantiomers thereof, and protein mimics thereof, unless explicitly described otherwise. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially, as well as fusion proteins containing a target molecule.

The term “physiological conditions” is meant to encompass those conditions compatible with living cells, e.g., predominantly aqueous conditions of a temperature, pH, salinity, etc. that are compatible with living cells.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

The term “nanobody” (Nb), as used herein, refers to the smallest antigen binding fragment or single variable domain (V_{HH}) derived from naturally occurring heavy chain antibody and is known to the person skilled in the art. A “nanobody” is a single-domain antibody, such as that generated from the variable heavy region of a human Ig or camelid Ig. A nanobody can be derived from heavy chain only antibodies, seen in camelids (Hamers-Casterman et al., (1993) Nature 363:446; Desmyter et al., (1996) Nature Struct. Biol. 3:803). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody. A “nanobody” is also referred to herein as a “single-domain antibody.”

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table 1 as a comparison.

TABLE 1
CDR Definitions

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of protein as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a target molecule” includes a plurality of such target molecules and reference to “the biomolecule” includes reference to one or more biomolecules and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a method for chemoselective modification of a cell surface molecule on a target cell. A subject method includes contacting a target cell comprising cell surface molecule comprising a thiol, an amine, or an imidazole with a biomolecule comprising a reactive moiety, wherein the reactive moiety is generated by reaction of a biomolecule (e.g., an antibody) comprising a phenol moiety or a catechol with an enzyme capable of oxidizing the phenol or the catechol moiety. The contacting is carried out under conditions sufficient for conjugation of the cell surface molecule to the biomolecule, thereby producing a modified cell. The present disclosure provides kits for carrying out a subject method. The present disclosure also provides modified cells and methods for using same. A modified cell of the present disclosure finds use in various research applications and various therapeutic applications.

### Methods

As summarized above, aspects of the present disclosure include a method for modification of a cell surface molecule (also referred to herein as a “target molecule”) on a target cell, e.g., on the surface of a target cell. The subject method includes contacting a target cell comprising a cell surface molecule (e.g., a cell surface polypeptide) comprising a thiol moiety, an amine moiety, or an imidazole moiety with a biomolecule comprising a reactive moiety, wherein the reactive moiety is generated by reaction of a biomolecule comprising a phenol moiety or a catechol moiety with an enzyme capable of oxidizing the phenol moiety or the catechol moiety. The contacting is carried out under conditions sufficient for conjugation of the cell surface molecule to the biomolecule, thereby producing a modified cell.

In some cases, a subject method for modification of a target cell comprises contacting: i) a cell comprising a cell surface molecule comprising a thiol moiety, an amine moiety, or an imidazole moiety; ii) a biomolecule comprising a phenol moiety or a catechol moiety; and iii) an enzyme capable of oxidizing the phenol or catechol moiety; wherein the enzyme oxidizes the phenol or catechol moiety of the biomolecule to generate a reactive moiety, thereby generating a biomolecule comprising the reactive moiety, and wherein the reactive moiety reacts with the thiol moiety, the amine moiety, or the imidazole moiety, thereby conjugating the cell surface molecule and the biomolecule to one another, thereby producing a modified cell. In some cases, the cell surface molecule comprises a single thiol moiety, a single amine moiety, or a single imidazole moiety. In some cases, the target molecule comprises two thiol moieties, two amine moieties, or two imidazole moieties.

The cell surface molecule can be any of a variety of molecules, but generally is a polypeptide. Similarly, the biomolecule can be any of a variety of molecules (e.g., polypeptides; nucleic acids; small molecules; etc.). In many instances, the biomolecule is an antibody.

Biomolecules of interest include, but are not limited to, polypeptides, polynucleotides, carbohydrates, lipids, fatty acids, steroids, purines, pyrimidines, derivatives, structural analogs thereof and combinations thereof. In certain instances, the biomolecule of interest is an antibody. In some instances, the biomolecule of interest is an antibody fragment or binding derivative thereof. In some cases, the antibody fragment or binding derivative thereof is selected from the group consisting of a Fab fragment, a F(ab′)2 fragment, a single-chain Fv (scFv), a diabody, a nanobody, and a triabody.

In certain embodiments, the biomolecule comprising a phenol or catechol moiety further comprises, one or more moieties selected from a fluorophore, an active small molecule, an affinity tag, and a metal-chelating agent (e.g., as described herein). In certain instances, the biomolecule of interest is a fluorescent protein. In certain cases, the fluorescent protein is a green fluorescent protein (GFP). In certain cases, the biomolecule is an enzyme. In certain cases, the biomolecule is a ligand for a receptor. In certain cases, the biomolecule is a receptor.

In some cases, the enzyme capable of oxidizing the phenol moiety or the catechol moiety is a phenol oxidase or a catechol oxidase. In certain cases, the enzyme is a tyrosinase.

The term “tyrosinase” is used herein to refer to monophenol monooxygenase (EC 1.14.18.1; CAS number: 9002-10-2), an enzyme that catalyzes the oxidation of phenols (such as tyrosine). It is a copper-containing enzyme present in plant and animal tissues that catalyzes the production of melanin and other pigments from tyrosine by oxidation. All tyrosinases have in common a binuclear type 3 copper center within their active site. Here two copper atoms are each coordinated with three histidine residues. Matoba et al., “Crystallographic evidence that the dinuclear copper center of tyrosinase is flexible during catalysis,” J Biol Chem. 2006 Mar. 31; 281(13):8981-90. Epub 2006 Jan. 25, disclose a three dimensional model of a tyrosinase catalytic center.

In certain embodiments, the subject enzyme is affixed to a solid support system, such as beads, resins, gels, microspheres, or other geometric configurations. In certain cases, the solid support is a glass bead. In some cases, the solid support is a resin bead. Use of an enzyme affixed to a solid support system can allow for multiple use of the subject enzyme, and can facilitate purification of modified cell by allowing for the enzyme to be easily removed from the reaction mixture. In certain embodiments, the subject enzyme affixed to a solid support system can allow for the subject methods to be carried out in a continuous flow system. In certain embodiments, the subject enzyme affixed to a solid support system can facilitate large batch processing of the subject methods.

In certain cases, the subject phenol moiety is present in a tyrosine residue. In certain cases, the tyrosine residue is part of the biomolecule of interest. In certain cases, the tyrosine residue is synthetically introduced into the biomolecule of interest. In some other cases, the tyrosine residue is linked to the biomolecule of interest via a linker (e.g., as described herein). A tyrosine residue can be introduced using standard recombinant techniques, e.g., by modifying a nucleotide sequence encoding a polypeptide biomolecule such that a tyrosine residue is introduced into the polypeptide biomolecule.

In some cases, a phenol or catechol moiety is part of an unnatural (non-genetically encoded) amino acid that is introduced into a biomolecule of interest. For example, amber codon (TAG) suppression can be used to incorporate a non-genetically encoded amino acid residue that comprises a phenol moiety or a catechol moiety. See, e.g., Chin et al. (2002) J. Am. Chem. Soc. 124:9026; Chin and Schultz (2002) Chem. Biol. Chem. 3:1135; Chin et al. (2002) Proc. Natl. Acad. Sci. USA 99:11020; U.S. 2015/0240249; and US 2018/0171321. As another example, an orthogonal RNA synthetase and/or an orthogonal tRNA can be used for introducing a non-genetically encoded amino acid into a biomolecule, where the non-genetically encoded amino acid comprises a phenol moiety or a catechol moiety.

In some embodiments of the subject methods, the thiol moiety present in the cell surface molecule is part of a cysteine residue. In certain cases, the cysteine residue is a native cysteine residue. In certain cases, the cysteine residue is a cysteine residue synthetically introduced into the cell surface molecule. In some embodiments of the subject methods, the amine moiety present in the cell surface molecule is part of a lysine residue. In certain cases, the lysine residue is a native lysine residue. In certain cases, the lysine residue is a lysine residue synthetically introduced into the cell surface molecule. In some embodiments of the subject methods, the imidazole moiety present in the cell surface molecule is part of a histidine residue. In certain cases, the histidine residue is a native histidine residue. In certain cases, the histidine residue is a residue synthetically introduced into the cell surface molecule.

In certain embodiments, the reactive moiety is an orthoquinone or a semi-quinone radical, or a combination thereof. In certain embodiments, the subject methods provide a reaction between an orthoquinone reactive intermediate and a thiol moiety, as depicted in Scheme 1 below:

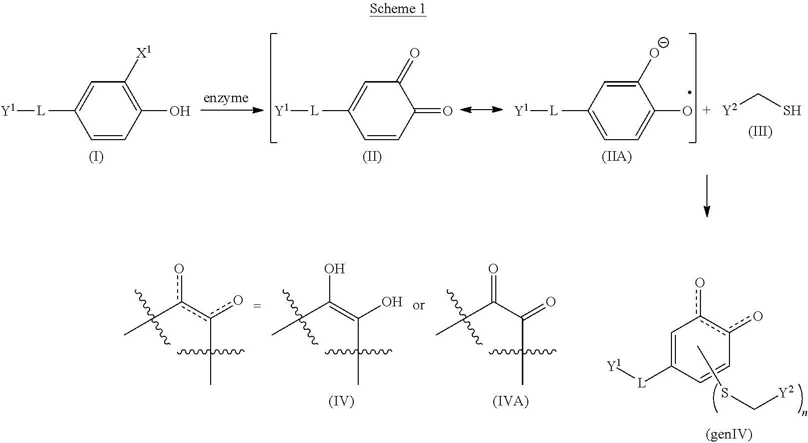

where Y¹ is any convenient biomolecule optionally comprising one or more moieties selected from, an active small molecule, a cleavable probe, a fluorophore, and a metal chelator; L is an optional linker (e.g., as described herein); X¹ is selected from hydrogen and hydroxyl; Y² is any convenient biomolecule; and n is an integer from 1 to 3.

In certain embodiments, the subject methods provide a reaction between an orthoquinone reactive intermediate and an amine moiety (e.g., a lysine residue present in a cell surface polypeptide), as depicted in Scheme 2 below:

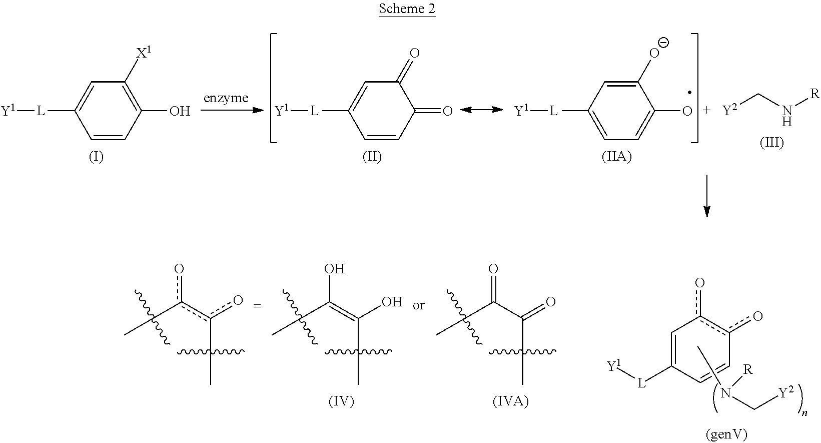

where Y¹ is any convenient biomolecule optionally comprising one or more moieties selected from, an active small molecule, a cleavable probe, a fluorophore, and a metal chelator; L is an optional linker (e.g., as described herein); X¹ is selected from hydrogen and hydroxyl; Y² is any convenient biomolecule; R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and n is an integer from 1 to 3. In certain instances, R is an amino acid selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine.

In certain embodiments, the subject methods provide a reaction between an orthoquinone reactive intermediate and an imidazole moiety (e.g., a histidine residue present in a cell surface molecule), as depicted in Scheme 3 below:

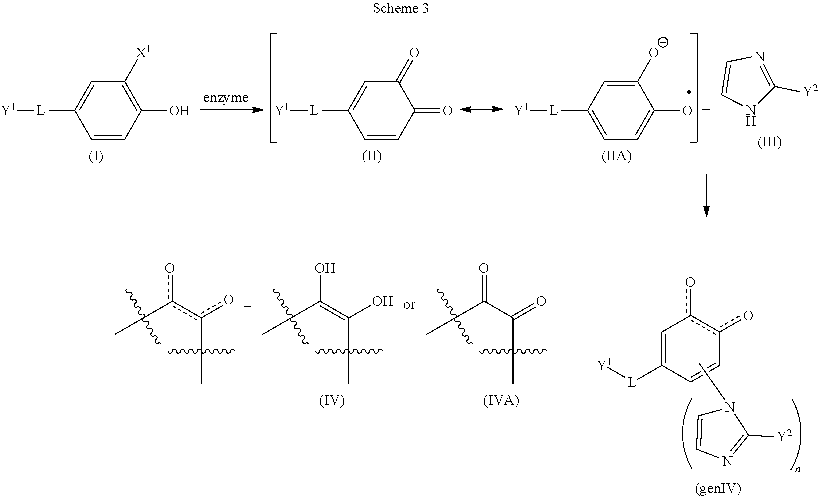

where Y¹ is any convenient biomolecule optionally comprising one or more moieties selected from, an active small molecule, a cleavable probe, a fluorophore, and a metal chelator; L is an optional linker (e.g., as described herein); X¹ is selected from hydrogen and hydroxyl; Y² is any convenient biomolecule; and n is an integer from 1 to 3.

In certain embodiments, the subject methods provide a reaction between an orthoquinone reactive intermediate and a nitrogen containing moiety (e.g., an amine or heterocyclic nitrogen-containing moiety such as an imidazole), as depicted in Scheme 4 below:

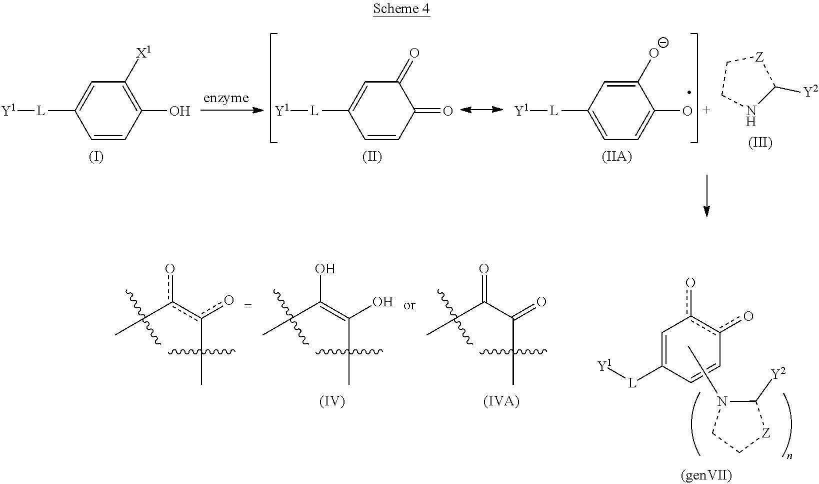

where Y¹ is any convenient biomolecule optionally comprising one or more moieties selected from, an active small molecule, a cleavable probe, a fluorophore, and a metal chelator; L is an optional linker (e.g., as described herein); X¹ is selected from hydrogen and hydroxyl; Y² is any convenient biomolecule; Z is selected from C, N, O or S and n is an integer from 1 to 3.

As depicted in Scheme 1, in certain embodiments, a biomolecule comprising a phenol or catechol moiety (e.g., of formula (I)), undergoes activation with an enzyme capable of oxidizing the phenol or catechol moiety. In some cases, activation is achieved with a tyrosinase enzyme in the presence of oxygen to generate an intermediate comprising a reactive moiety (e.g., orthoquinone of formula (II) and/or semi-quinone radical of formula (IIA)), and the said reactive moiety reacts with a cell surface molecule comprising a thiol based nucleophile (e.g., of formula (III)), to result in conjugation of the cell surface molecule to the biomolecule, thereby producing a modified cell surface molecule (e.g., of formula (genIV)). In certain embodiments, a target molecule of formula (III) may comprise any convenient biomolecule, e.g., as described herein. In certain cases, Y² in formula (III) is a polypeptide. In certain cases, the modified molecule is described by the formula (IV). In some cases, the modified cell surface molecule is described by the formula (IVA). In certain cases, the modified cell surface molecule is described by any one of formulae (IV)-(IVL), as described herein.

In certain embodiments, the subject methods provide a reaction between an orthoquinone reactive intermediate and a thiol moiety, as depicted in Scheme 5 below:

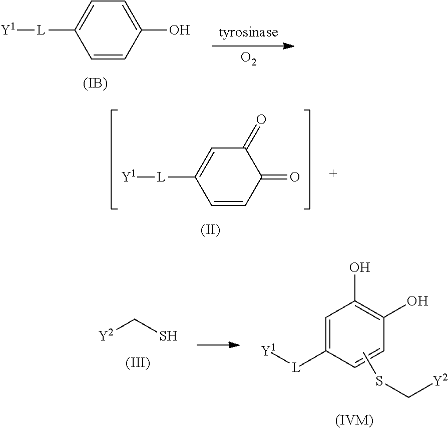

As depicted in Scheme 2, in certain embodiments, a biomolecule comprising a phenol moiety (e.g., of formula (IB)) undergoes activation with a tyrosinase enzyme in the presence of oxygen to generate an intermediate comprising a reactive moiety (e.g., orthoquinone of formula (II)), and the said reactive moiety reacts with a cell surface molecule comprising a thiol based nucleophile (e.g., of formula (III)), to result in conjugation of the cell surface molecule to the biomolecule, thereby producing a modified cell surface molecule (e.g., of formula (IVM). In certain embodiments, a target molecule of formula (III) may comprise any convenient biomolecule, e.g., as described herein. In certain cases, Y² in formula (III) is a polypeptide. In certain cases of the modified molecule of formula (IVM), the thiol group is at the 3-position of the catechol ring. In certain cases of the modified molecule of formula (IVM), the thiol group is at the 5-position of the catechol ring. In certain cases of the modified molecule of formula (IVM), the thiol group is at the 6-position of the catechol ring.

In certain embodiments, the subject methods provide a reaction between an orthoquinone reactive intermediate and an amine moiety, as depicted in Scheme 6 below:

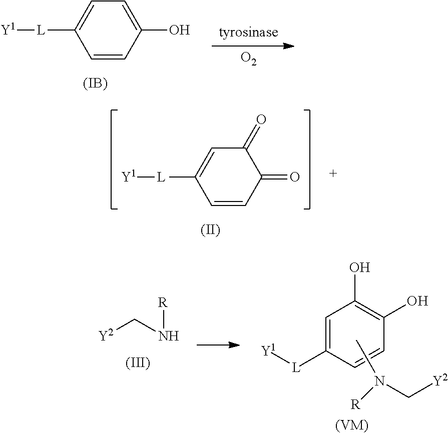

In certain embodiments, the subject methods provide a reaction between an orthoquinone reactive intermediate and an imidazole moiety, as depicted in Scheme 7 below:

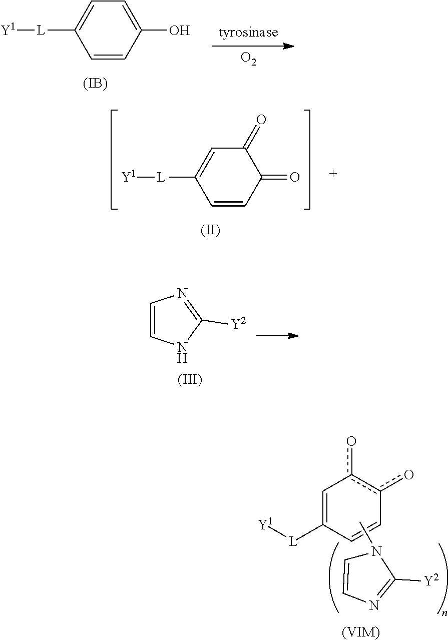

In certain embodiments, the biomolecule of formula (I) may be any one of formulae (IA)-(IDb), e.g., as described herein and discussed in more detail below. In certain embodiments, the modified cell surface molecule may be of any one of formulae (IV)-(IVL), e.g., as described herein and discussed in more detail below. In certain embodiments, the modified cell surface molecule is a product of a single conjugation, e.g., as shown in formulae (IV1)-(IV3), (IVA1)-(IVA3), (IVB1)-(IVB3), (IVC1)-(IVC3), (IVD1)-(IVD3), (IVE1)-(IVE3), (IVF1)-(IVF3), (IVG1)-(IVG3), (IVH1)-(IVH3) and (IVJ1)-(IVJ3). In certain cases, the modified cell surface molecule is a product of double conjugation, e.g., as shown in formulae (IV4)-(IV5), (IVA4)-(IVA5), (IVB4)-(IVB5), (IVC4)-(IVC5), (IVD4)-(IVD5), (IVE4)-(IVE5), (IVF4)-(IVF5), (IVG4)-(IVG5), (IVH4)-(IVH5) and (IVJ4)-(IVJ5).

In certain embodiments, the modified cell surface molecule may be of any one of formulae (V)-(VL), e.g., as described herein and discussed in more detail below. In certain embodiments, the modified cell surface molecule is a product of a single conjugation, e.g., as shown in formulae (V1)-(V3), (VA1)-(VA3), (VB1)-(VB3), (VC1)-(VC3), (VD1)-(VD3), (VE1)-(VE3), (VF1)-(VF3), (VG1)-(VG3), (VH1)-(VH3) and (VJ1)-(VJ3). In certain cases, the modified cell surface molecule is a product of double conjugation, e.g., as shown in formulae (V4)-(V5), (VA4)-(VA5), (VB4)-(VB5), (VC4)-(VC5), (VD4)-(VD5), (VE4)-(VE5), (VF4)-(VF5), (VG4)-(VG5), (VH4)-(VH5) and (VJ4)-(VJ5).

In certain embodiments, the modified cell surface molecule may be of any one of formulae (VI)-(VIIL), e.g., as described herein and discussed in more detail below. In certain embodiments, the modified cell surface molecule is a product of a single conjugation, e.g., as shown in formulae (VI)-(VI3), (VIA1)-(VIA3), (VIB1)-(VIB3), (VIC1)-(VIC3), (VID1)-(VID3), (VIE1)-(VIE3), (VIF1)-(VIF3), (VIG1)-(VIG3), (VIH1)-(VIH3) and (VIJ1)-(VIJ3). In certain cases, the modified cell surface molecule is a product of double conjugation, e.g., as shown in formulae (V14)-(VI5), (VIA4)-(VIA5), (VIB4)-(VIB5), (VIC4)-(VIC5), (VID4)-(VID5), (VIE4)-(VIE5), (VIF4)-(VIF5), (VIG4)-(VIG5), (VIH4)-(VIH5) and (VIJ4)-(VIJ5).

In certain embodiments, the subject method is carried out at a pH from 4 to 9, such as 4.2, 4.5, 4.8, 5.0, 5.2, 5.5, 5.8, 6.0, 6.2, 6.5, 6.8, 7.0, 7.2, 7.5, 7.8, 8.0, 8.2, 8.5, 8.8 or 9. In certain embodiments, the subject method is carried out at a pH of from 5 to 8, such as 5.2, 5.5, 5.8, 6.0, 6.2, 6.5, 6.8, 7.0, 7.2, 7.5, 7.8 or 8.0. In certain cases, the subject method is carried out at a pH of 6 to 7.5, such as 6.0, 6.3, 6.4, 6.5, 6.6, 6.8, 7.0, 7.2, 7.4, or 7.5. In certain embodiments, the subject method is carried out at neutral pH. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain embodiments, the subject methods may be carried out under physiological conditions. In some cases, the method is carried out on living cells in vitro. In other instances, the method is carried out on living cells ex vivo.

In certain embodiments, the subject methods may be carried out in aqueous media in the presence of one or more buffers. Buffers of interest include, but are not limited to, a phosphate buffer, 2-amino-2-(hydroxymethyl)propane-1,3-diol (TRIS), 4-[4-(2-hydroxyethyl)piperzin-1-yl]ethanesulfonic acid (HEPES), and the like. In certain embodiments, the subject methods may be carried out in an organic solvent. In certain cases, the organic solvent is a water miscible solvent. In certain cases, the organic solvent is a dipolar aprotic solvent. In certain cases, the organic solvent is selected from acetonitrile, dimethyl formamide, methanol and acetone. In certain cases, the organic solvent is present in an amount from 1 to 20%, relative to water, such as 2%, 5%, 10%, 15% or 20%. In some cases, the subject method is carried out in from 1% to 20% acetonitrile, such as 5%, 10%, 15% or 20%. In some cases, the subject method is carried out in from 1% to 20% dimethyl formamide, such as 5%, 10%, 15%, or 20%. In some cases, the subject method is carried out in from 1% to 20% methanol, such as 5%, 10%, 15%, or 20%. In some cases, the subject method is carried out in from 1% to 20% acetone, such as 5%, 10%, 15%, or 20%.

In certain embodiments of the subject methods, the modified cell surface molecule is a product of double or triple conjugation (e.g., referring to formula (IV), when n is 2 or 3, referred to collectively herein as “multiple conjugation products”). In certain embodiments of the subject methods, multiple conjugation products are present in less than 1 part in 10 by weight of one or more multiple conjugation products relative to the single conjugation product (e.g., referring to formula (IV), when n is 1), such as less than 1 part in 20, less than 1 part in 25, less than 1 part in 50, less than 1 part in 75, less than 1 part in 100, or even less. In certain embodiments of the subject methods, no multiple conjugation products are observed.

In certain embodiments of the methods, the modified cell surface molecule is stable at a range of pH and temperature values and in the presence of a number of additional molecules. In some cases, the modified target molecule is stable from 0° C. to 50° C., such as 4° C. to 40° C., such as 4° C. to 37° C. In certain cases, the modified target molecule is stable over a pH range of 4 to 9, such as at pH 4, 4.5, 5, 5.5, 6, 6.5, 7, 7.5, 8, 8.5 or 9. In certain cases, the modified cell surface molecule is stable in the presence of biologically relevant molecules. In some cases, the modified cell surface molecule is stable in physiological conditions; for example, in some cases, the modified target molecule is stable in human serum. In some cases, the modified target molecule remains on the surface of a modified target cell for at least 2 hours, at least 4 hours, at least 8 hours, at least 12 hours, at least 24 hours, at least 2 days, at least 3 days, at least 4 days, at least 5 days, at least 6 days, at least 7 days, at least 10 days, or at least 14 days.

In some cases, a method of the present disclosure for modifying a target cell comprises contacting, in vitro, from about 10² to about 10⁸ unmodified target cells (e.g., from about 10² to 10³, from about 10³ to about 10⁴, from about 10⁴ to about 10⁵, from about 10⁵ to about 10⁶, from about 10⁶ to about 10⁷, or from about 10⁷ to about 10⁸ unmodified target cells) with a biomolecule (e.g., an antibody, such as a scFv or a nanobody) comprising a tyrosine, where the biomolecule is present in a concentration of from about 1 μM to about 25 μM (e.g., from about 1 μM to about 5 μM, from about 5 μM to about 10 μM, from about 10 μM to about 15 μM, or from about 15 μM to about 25 μM; and b) a tyrosinase polypeptide, where the tyrosinase polypeptide is present in a concentration of from about 100 nM to about 500 nM (e.g., from about 100 nM to about 200 nM, from about 200 nM to about 300 nM, from about 300 nM to about 400 nM, or from about 400 nM to about 500 nM). The reaction can be carried out in a liquid medium under standard cell culture conditions, for a period of time of from about 1 minute to about 15 minutes (e.g., from about 1 minute to about 5 minutes, from about 5 minutes to about 10 minutes, or from about 10 minutes to about 15 minutes).

In some cases, a method of the present disclosure provides for conjugation of from about 10² to about 5×10⁵ copies of an antibody (e.g., a nanobody or a scFv) to a cell. For example, in some cases, a modified cell generated using a method of the present disclosure comprises from about 10² to about 10³, from about 10³ to about 10⁴, from about 10⁴ to about 5×10⁴, from about 5×10⁴ to about 10⁵, from about 10⁵ to about 2×10⁵, or from about 2×10⁵ to about 5×10⁵, copies of an antibody per cell. In some cases, a modified cell generated using a method of the present disclosure comprises about 100,000 copies of an antibody per cell. In some cases, a modified cell generated using a method of the present disclosure comprises about 120,000 copies of an antibody per cell.

### Tyrosinase Polypeptides

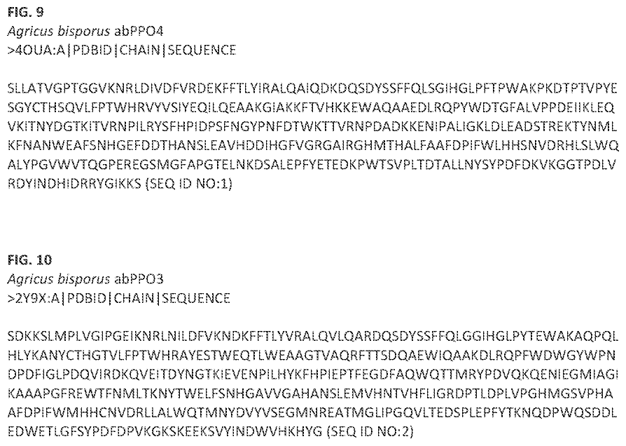

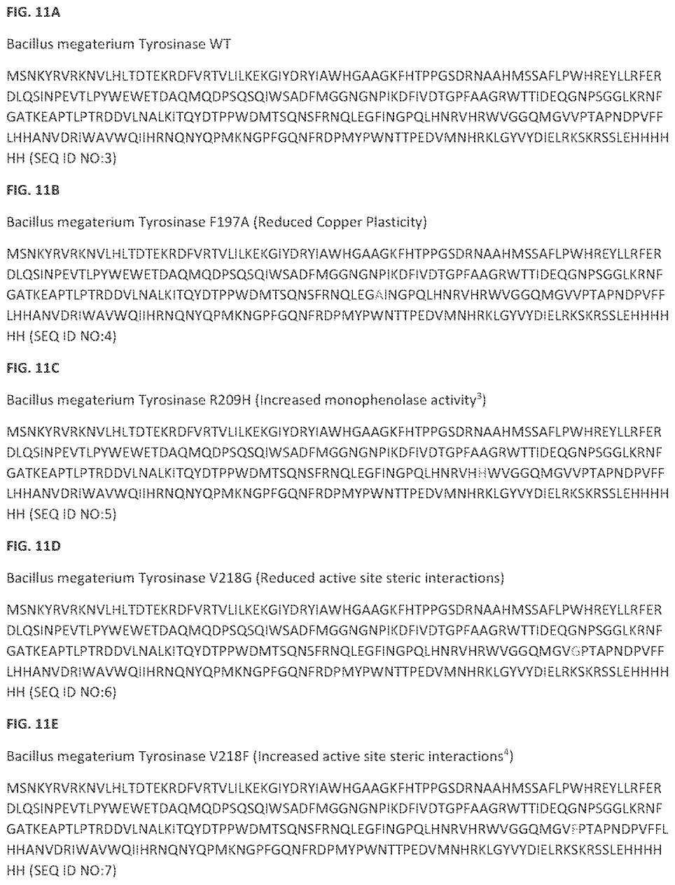

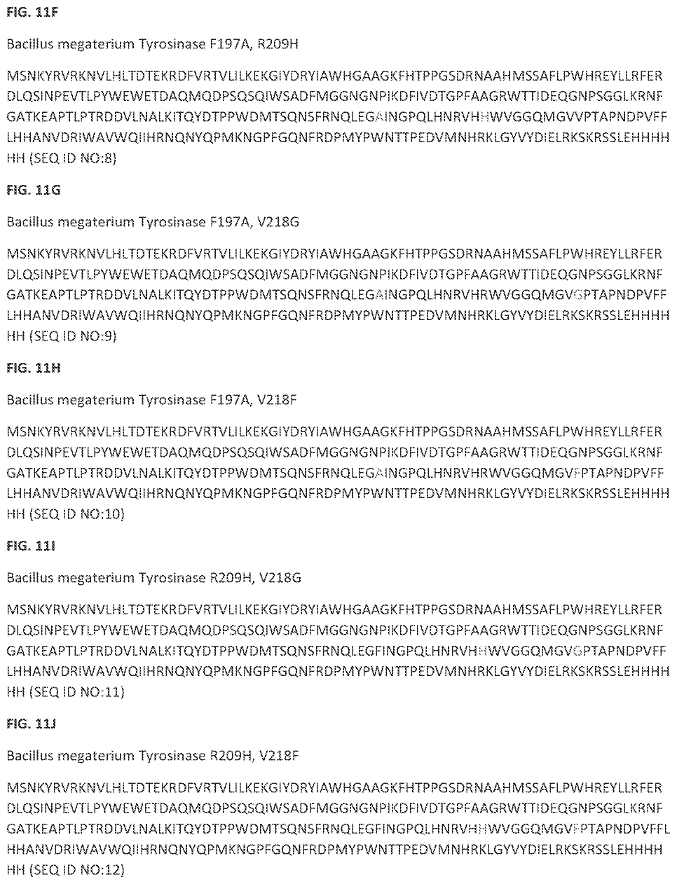

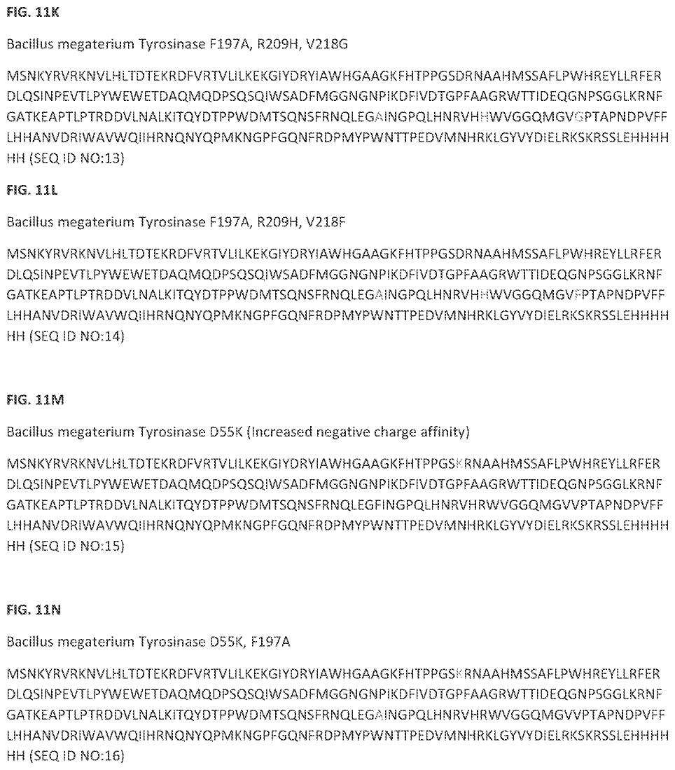

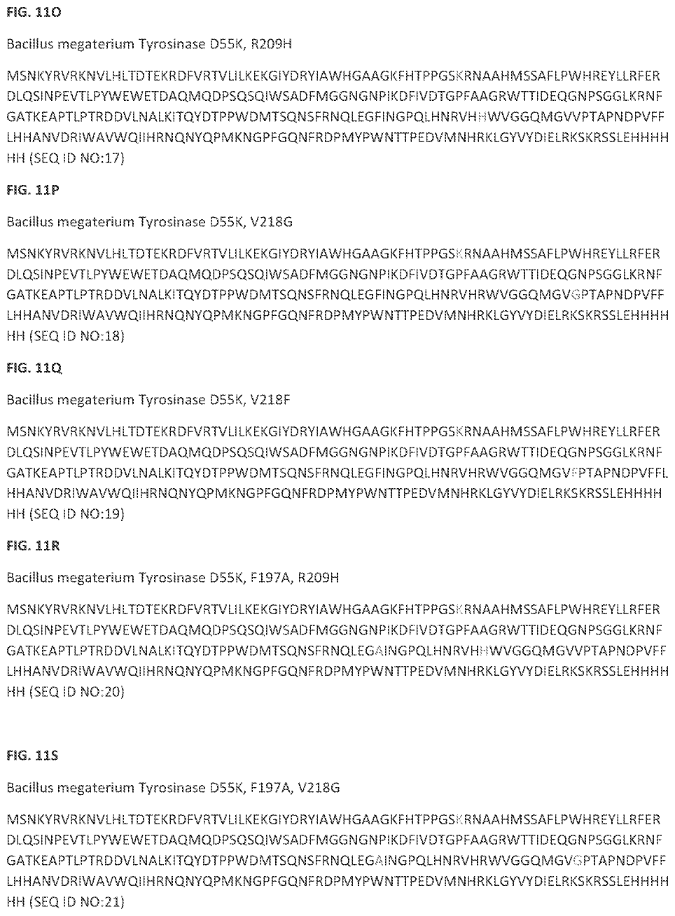

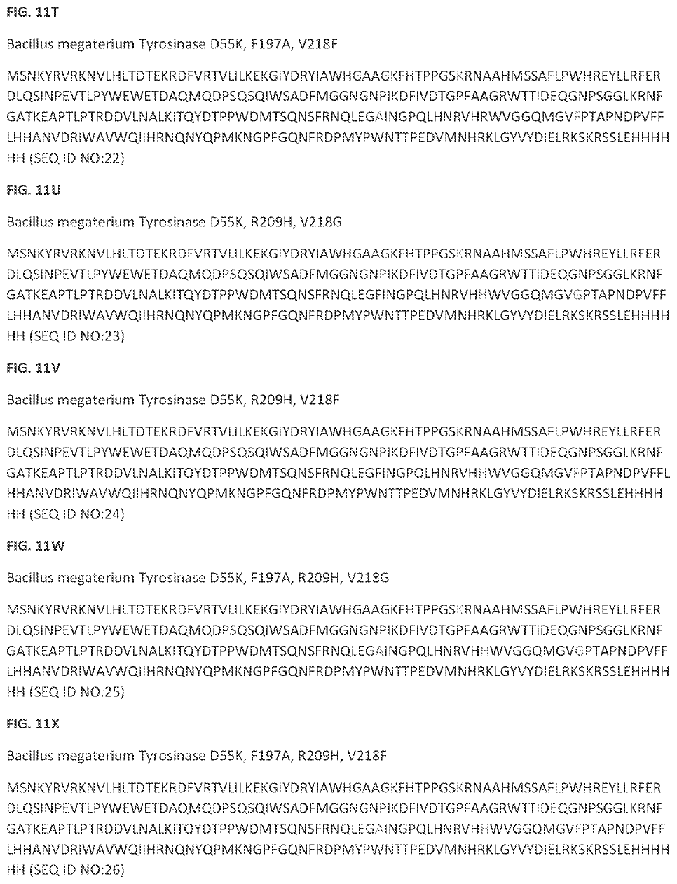

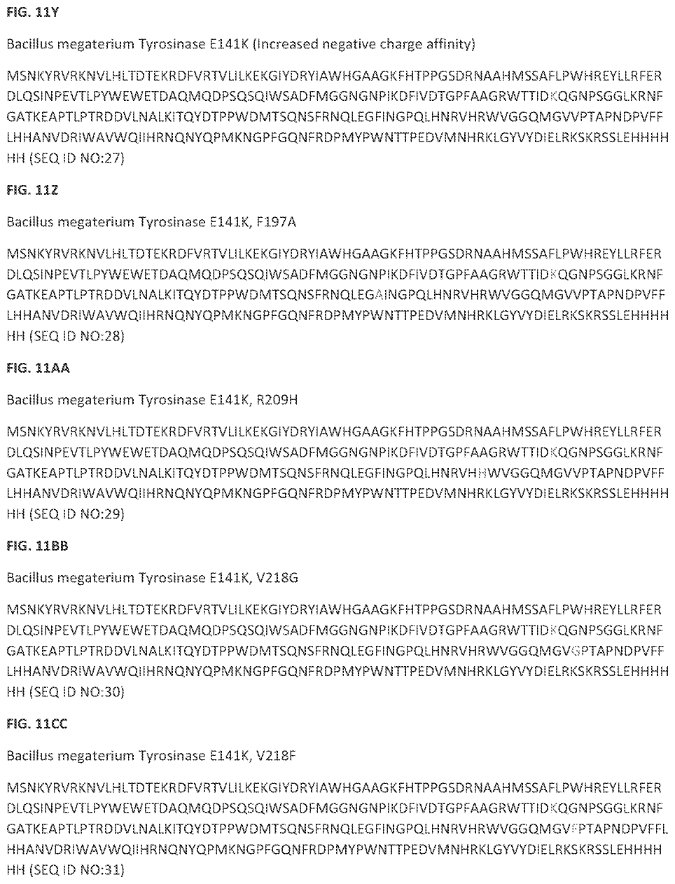

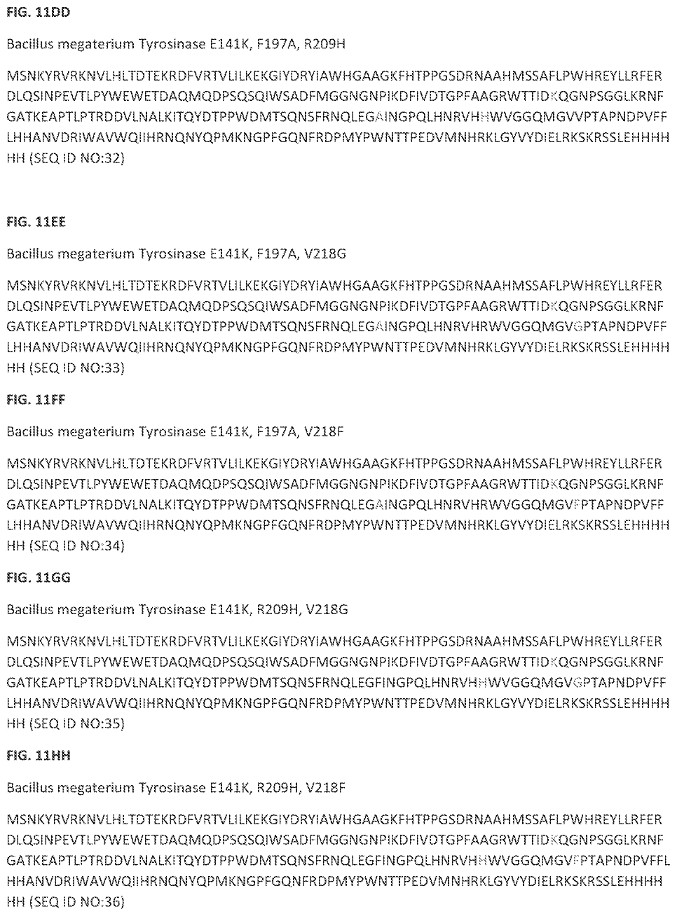

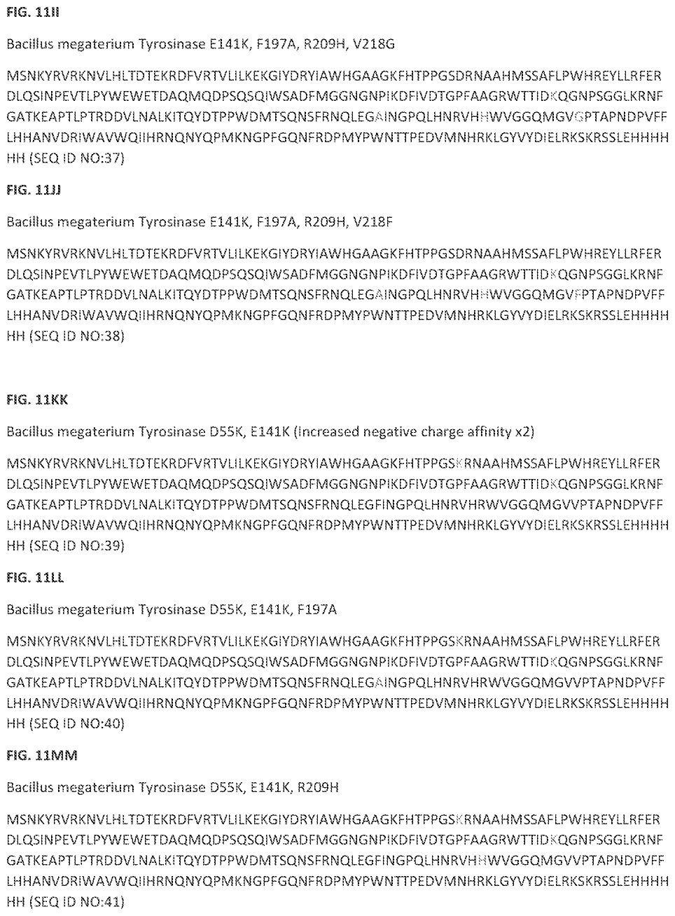

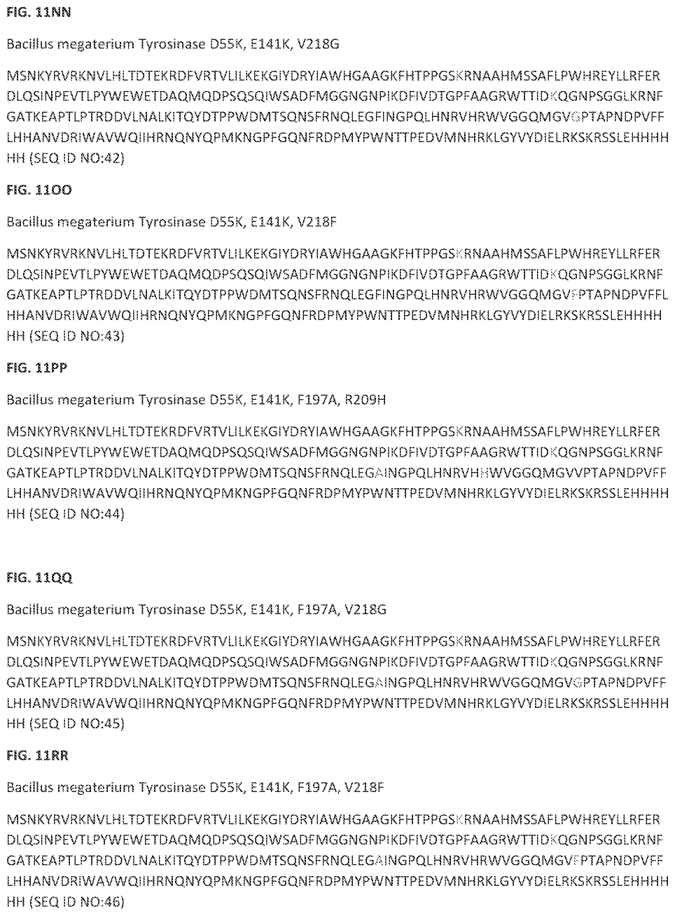

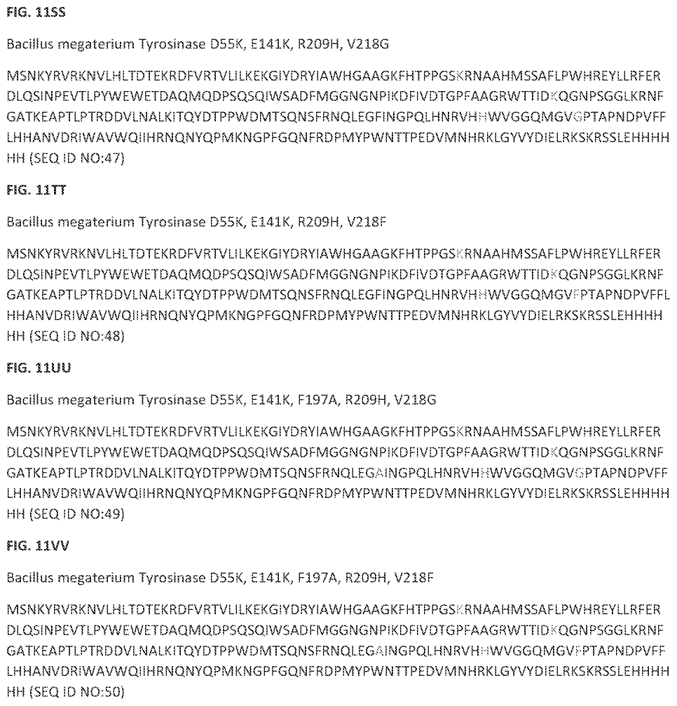

Tyrosinase polypeptides that are suitable for use in generating a reactive moiety (e.g., an orthoquinone) include a tyrosinase polypeptide having at least 75%, at least 80%, at least 85%, at least 90%, at least 95%, at least 98%, at least 99%, or 100%, amino acid sequence identity to any one of the tyrosinase amino acid sequences set forth in FIG. 9, FIG. 10, FIG. 11A-11Z, and FIG. 11AA-11VV. In some cases, the tyrosinase polypeptide is an Agricus bisporus tyrosinase polypeptide. In some cases, the tyrosinase polypeptide is a Bacillus megaterium tyrosinase polypeptide. In some cases, the tyrosinase polypeptide is a Streptomyces castaneoglobisporus tyrosinase polypeptide. In some cases, the tyrosinase polypeptide is a Citrobacter freundii tyrosinase polypeptide. In some cases, the tyrosinase polypeptide is a Homo sapiens tyrosinase polypeptide. In some cases, the tyrosinase polypeptide is a Malus domestica tyrosinase polypeptide. In some cases, the tyrosinase polypeptide is an Aspergillus oryzae tyrosinase polypeptide. In some cases, the tyrosinase polypeptide is a Solanum lycopersicum tyrosinase polypeptide. In some cases, the tyrosinase polypeptide is a Burkholderia thailandensis tyrosinase polypeptide. In some cases, the tyrosinase polypeptide is a Juglans regia tyrosinase polypeptide. See, e.g., Pretzler et al. Sci. Rep. 2017, 7 (1), 1810; Ren et al. BMC Biotechnol. 2013, 13, 18; Faccio et al. Process Biochem. 2012, 47 (12), 1749-1760; Fairhead et al. FEBS J. 2010, 277 (9), 2083-2095; Do et al. Sci. Rep. 2017, 7 (1), 17267; Elsayed and Danial J. Appl. Pharm. Sci. 2018, 8 (09), 93-101; Lopez-Tejedor and Palomo Protein Expr. Purif. 2018, 145, 64-70; and Fairhead et al. Nature Biotechnol. 2012, 29 (2), 183-191.

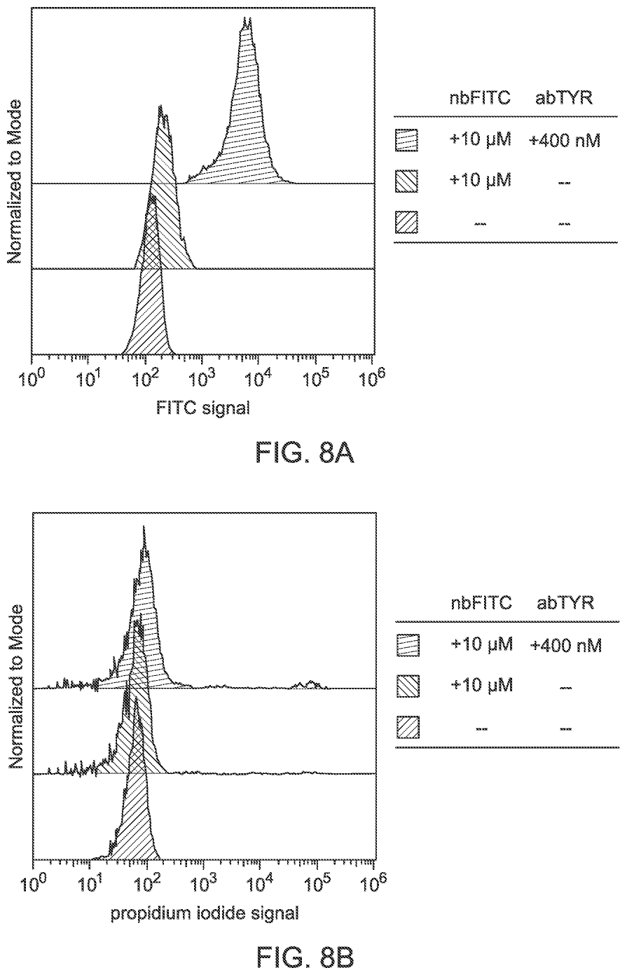

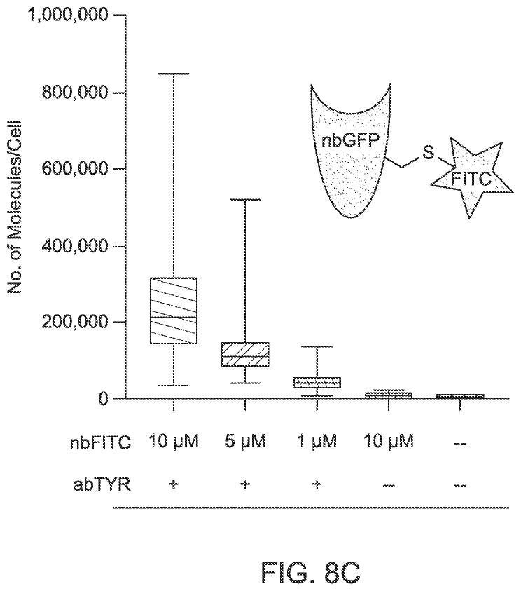

In some cases, the tyrosinase polypeptide selectively acts on (e.g., generates a reactive moiety such as an orthoquinone) a substrate (a biomolecule) comprising a phenol moiety (e.g., a tyrosine) or a catechol moiety, where the substrate is neutral or positively charged within 50 Å (e.g., within 50 Å, within 40 Å, within 30 Å, or within 20 Å) of the phenol or the catechol moiety. For example, a tyrosinase having at least 75%, at least 80%, at least 85%, at least 90%, at least 95%, at least 98%, at least 99%, or 100%, amino acid sequence identity to any one of the tyrosinase amino acid sequences set forth in FIG. 8 or FIG. 9 can selectively modify a phenol or catechol moiety on a substrate, where the substrate is neutral or positively charged within 50 Å (e.g., within 50 Å, within 40 Å, within 30 Å, or within 20 Å) of the phenol or the catechol moiety. For example, where the biomolecule is a polypeptide, in some cases, the biomolecule comprises at least 2 neutral or positively charged amino acids within 10 amino acids of the phenol moiety (e.g., a tyrosine) or a catechol moiety. For example, where the biomolecule is a polypeptide, in some cases, the biomolecule comprises 2, 3, 4, 5, 6, 7, 8, 9, or 10 neutral or positively charged amino acids within 10 amino acids of the phenol moiety (e.g., a tyrosine) or a catechol moiety.

In some cases, the tyrosinase polypeptide selectively acts on (e.g., generates a reactive moiety such as an orthoquinone) a substrate (a biomolecule) comprising a phenol moiety (e.g., a tyrosine) or a catechol moiety, where the substrate is negatively charged within 50 Å (e.g., within 50 Å, within 40 Å, within 30 Å, or within 20 Å) of the phenol or the catechol moiety. For example, a tyrosinase having at least 75%, at least 80%, at least 85%, at least 90%, at least 95%, at least 98%, at least 99%, or 100%, amino acid sequence identity to any one of the tyrosinase amino acid sequences set forth in FIG. 11A-11Z and FIG. 11AA-11VV can selectively modify a phenol or catechol moiety on a substrate, where the substrate is negatively charged within 50 Å (e.g., within 50 Å, within 40 Å, within 30 Å, or within 20 Å) of the phenol or the catechol moiety. For example, where the biomolecule is a polypeptide, in some cases, the biomolecule comprises at least 2 negatively charged amino acids within 10 amino acids of the phenol moiety (e.g., a tyrosine) or a catechol moiety. For example, where the biomolecule is a polypeptide, in some cases, the biomolecule comprises 2, 3, 4, 5, 6, 7, 8, 9, or 10 negatively charged amino acids within 10 amino acids of the phenol moiety (e.g., a tyrosine) or a catechol moiety.

In some cases, the tyrosinase polypeptide comprises an amino acid sequence having at least 75%, at least 80%, at least 85%, at least 90%, at least 95%, at least 98%, at least 99%, or 100%, amino acid sequence identity to the tyrosinase amino acid sequence depicted in FIG. 11M, where the tyrosinase polypeptide comprises an amino acid substitution of D55, e.g., where D55 is substituted with a Lys.

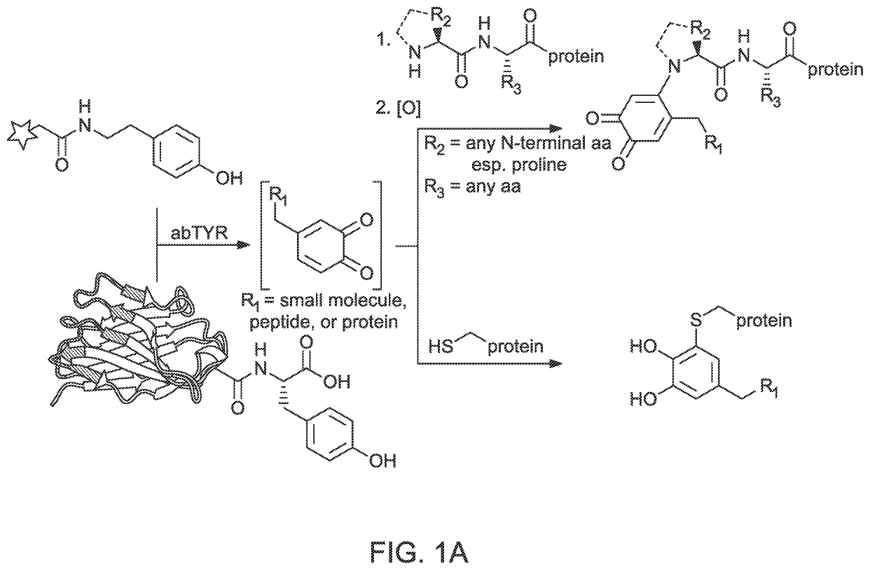

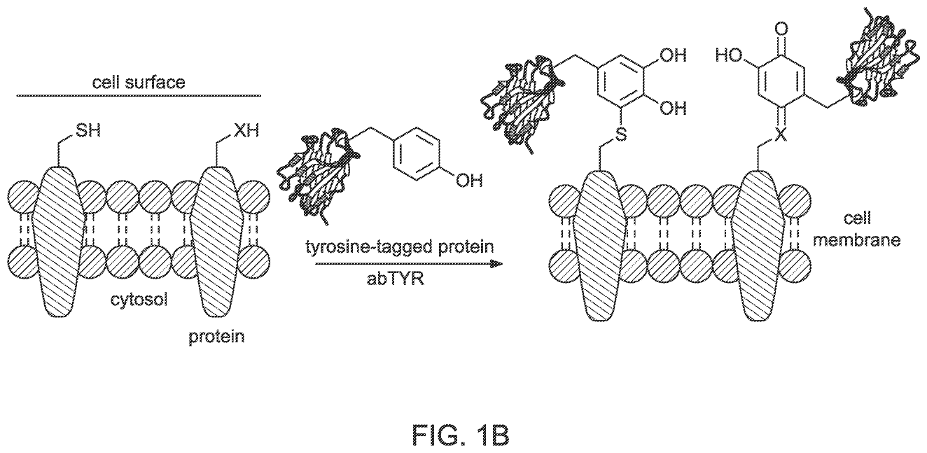

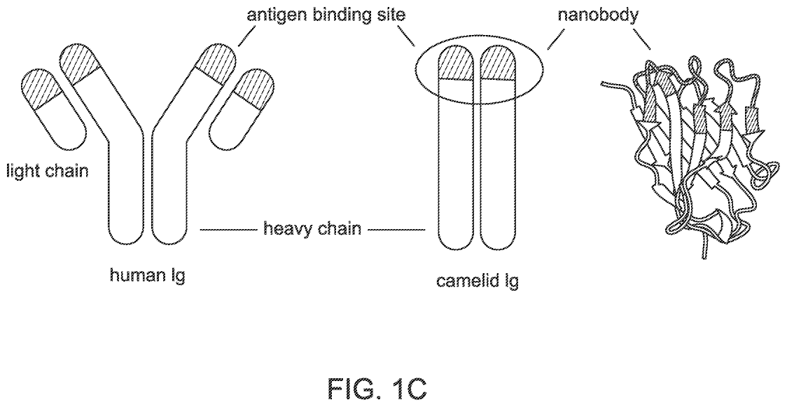

In some cases, the tyrosinase polypeptide comprises an amino acid sequence having at least 75%, at least 80%, at least 85%, at least 90%, at least 95%, at least 98%, at least 99%, or 100%, amino acid sequence identity to the tyrosinase amino acid sequence depicted in FIG. 1 IC, where the tyrosinase polypeptide comprises an amino acid substitution of R²⁰⁹, e.g., where R²⁰⁹ is substituted with a His.

### Cell Surface Modification

In some embodiments, a subject method is used to modify the surface of a cell with a biomolecule (e.g., an antibody, such as a scFv or a nanobody). Thus, in one aspect, the present disclosure features a method of modifying the surface of cell in vitro. The method generally involves reacting a thiol moiety (e.g., a thiol moiety present in a cysteine residue), an amine moiety (e.g., an amine moiety present in a lysine residue), or an imidazole moiety (e.g., an imidazole moiety present in a histidine residue) present in a cell surface molecule on the surface of an unmodified target cell with a biomolecule comprising a reactive moiety to provide for conjugation of the biomolecule (e.g., antibody) at the cell surface.

Suitable cells include immune cells, e.g., natural killer (NK) cells, T cells (e.g., cytotoxic T cells, such as CD8⁺ T cells), and the like. In some cases, the immune cells are obtained from an individual having a cancer. Suitable cells include, but are not limited to, a macrophage, a dendritic cell, a mast cell, an cosinophil, a basophil, a neutrophil, and a monocyte. In some cases, the cll is a T cell. In some cases, the cell is a B cell. In some cases, the T cell is selected from the group consisting of a CD4⁺ cell, CD8⁺ cell, a helper T cell, a cytotoxic T cell, and a regulatory T cell.

In some cases, the cell is a stem cell. In some cases, the cell is a hematopoietic stem cell. In some cases, the cell is a pluripotent stem cell. In some cases, the cell is a differentiated stem cell. In some cases, the cell is an induced pluripotent stem cell.

### Attachment of Biomolecules for Delivery to a Target Site

The biomolecule comprising a reactive moiety will in some embodiments comprise a small molecule drug, toxin, or other molecule for delivery to a cell. The small molecule drug, toxin, or other molecule will in some embodiments provide for a pharmacological activity. The small molecule drug, toxin, or other molecule will in some embodiments serve as a target for delivery of other molecules.

Small molecule drugs may be small organic or inorganic compounds having a molecular weight of more than 50 and less than about 2,500 daltons. Small molecule drugs may comprise functional groups necessary for structural interaction with proteins, particularly hydrogen bonding, and may include at least an amine, carbonyl, hydroxyl or carboxyl group, and may contain at least two of the functional chemical groups. The drugs may comprise cyclical carbon or heterocyclic structures and/or aromatic or polyaromatic structures substituted with one or more of the above functional groups. Small molecule drugs are also found among biomolecules including peptides, saccharides, fatty acids, steroids, purines, pyrimidines, derivatives, structural analogs or combinations thereof.

In another embodiment, a subject biomolecule comprising a reactive moiety comprises one of a pair of binding partners (e.g., a ligand; a ligand-binding portion of a receptor; an antibody; an antigen-binding fragment of an antibody; an antigen; a hapten; a lectin; a lectin-binding carbohydrate; etc.). For example, the biomolecule can comprise a polypeptide that serves as a viral receptor and, upon binding with a viral envelope protein or viral capsid protein, facilitates attachment of virus to the cell surface on which the biomolecule is displayed. Alternatively, the biomolecule comprises an antigen that is specifically bound by an antibody (e.g., monoclonal antibody), to facilitate detection and/or separation of host cells displaying the antigen on the cell surface. In another example, the biomolecule comprises a ligand binding portion of a receptor, or a receptor-binding portion of a ligand.

### Compounds

### Biomolecule Comprising a Phenol Moiety or a Catechol Moiety

In certain embodiments of the subject methods, the biomolecule comprising a phenol moiety or a catechol moiety is described by the formula (I).

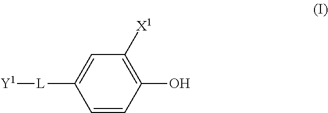

where Y¹ is a biomolecule, optionally comprising one or more moieties selected from, an active small molecule, an affinity tag, a fluorophore, and a metal-chelating agent; X¹ is selected from hydrogen and hydroxyl; and L is an optional linker.

In certain embodiments of formula (I), X¹ is hydrogen, such that the biomolecule comprises a phenol moiety. In other embodiments of formula (I), X¹ is hydroxyl, such that the biomolecule comprises a catechol moiety.

In some embodiments of formula (I), the phenol moiety is present in a tyrosine residue. In certain cases, the biomolecule comprising a phenol moiety of formula (I), is of the formula (IB) or (IC):

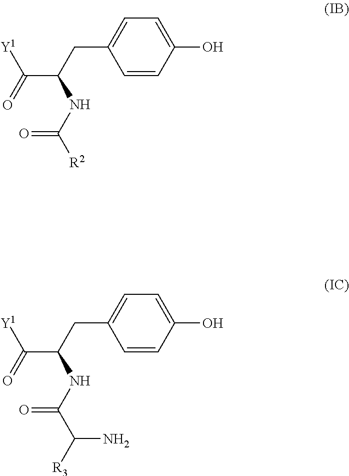

wherein R² is selected from alkyl, and substituted alkyl; and R³ is selected from hydrogen, alkyl, substituted alkyl, a peptide, and a polypeptide.

In certain embodiments of the subject methods, the biomolecule comprising a phenol moiety or a catechol (e.g., of formula (I)) includes a linker (e.g., as described herein). Suitable linkers include, but are not limited to, a carboxylic acid, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone, a halosulfonyl, a nitrile, a nitro, a PEG, and a peptide linker.

Exemplary linkers for use in linking the phenol moiety to the subject biomolecule of interest will in some embodiments include a PEG linker. As used herein the term “PEG” refers to a polyethylene glycol or a modified polyethylene glycol. Modified polyethylene glycol polymers include a methoxypolyethylene glycol, and polymers that are unsubstituted or substituted at one end with an alkyl, a substituted alkyl or a functional group (e.g., as described herein). Any convenient linking groups may be utilized at the terminal of a PEG to connect the group to a moiety of interest including but not limited to, alkyl, aryl, hydroxyl, amino, acyl, acyloxy, carboxyl ester and amido terminal and/or substituent groups. In certain instances, the linker includes more than 1 PEG unit, such as 2, 3, 4, 5, 6, 7, 8, 9 or 10 PEG units. In certain instances, the linker includes less than 10 PEG units, such as 9, 8, 7, 6, 5, 4, 3, 2 or 1 PEG unit. In certain cases, linker is composed of 4 or fewer PEG units.

In certain cases, the biomolecule comprising a phenol moiety is described by the formula (IA):

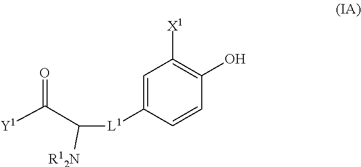

wherein:
Y¹ is a biomolecule optionally comprising one or more groups selected from, an active small molecule, an affinity tag, a fluorophore, and a metal-chelating agent;
each R¹ is independently selected from hydrogen, acyl, substituted acyl, alkyl, and substituted alkyl;
X¹ is selected from hydrogen and hydroxyl; and
L¹ is a linker selected from a straight or branched alkyl, a straight or branched substituted alkyl, a polyethylene glycol (PEG), a substituted PEG, and one or more peptides.

In certain embodiments, X¹ is hydrogen, such that the compound of formula (IA) is of the formula (IAa):

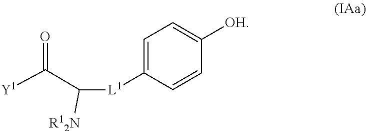

In certain embodiments of any of formulae (IA)-(IAa), at least one R¹ is hydrogen. In certain cases, both R¹ groups are hydrogen. In certain cases, one R¹ group is hydrogen, and the other R¹ group is selected from alkyl, substituted alkyl, acyl and substituted acyl. In certain cases, one R¹ group is hydrogen and the other R¹ group is alkyl. In some cases, one R¹ group is hydrogen and the other R¹ group is substituted alkyl. In some cases, one R¹ group is hydrogen and the other R¹ group is acyl. In some cases, one R¹ group is hydrogen, and the other R¹ group is substituted acyl. In some cases the acyl group is of the formula —C(O)R⁴, wherein R⁴ is a lower alkyl group, e.g., methyl, ethyl, propyl, butyl, pentyl, or hexyl. In some cases, the substituted acyl group is of the formula —C(O)R⁴NH₂, wherein R⁴ is a lower alkyl group, e.g., methyl, ethyl, propyl, butyl, pentyl or hexyl. In some cases, the substituted acyl group is of the formula —C(O)CH₂NH₂.

In certain embodiments of any of formulae (IA)-(IAa), L¹ is a straight or branched alkyl. In certain cases, L¹ is a lower alkyl group, e.g., methyl, ethyl, propyl, butyl, pentyl, or hexyl. In certain cases, L¹ is a substituted alkyl group. In certain cases, L¹ is a substituted lower alkyl group. In certain cases, L¹ is a PEG or substituted PEG (e.g., as described herein). In certain other cases, L¹ is a peptide. In certain other cases, L¹ is a polypeptide. In certain cases, L¹ is a linear linker of 1-12 atoms in length, such as 1-10, 1-8 or 1-6 atoms in length, e.g., 1, 2, 3, 4, 5 or 6 atoms in length. The linker L¹ can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl). In certain cases, the linker L¹ can include a keto (C═O) group. In certain cases, the keto group together with an amino, thiol or ether group in the linker chain can provide an amido, an ester or thioester group linkage.

In certain embodiments, the linking group L or L¹ is a cleavable linker, e.g., as described herein.

In certain embodiments, the biomolecule comprising a phenol or catechol moiety is described by the formula (ID):

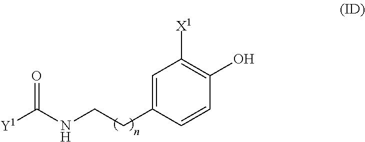

where Y¹ is a biomolecule optionally comprising one or more groups selected from, an active small molecule, an affinity tag, a fluorophore, and a metal-chelating agent; X¹ is selected from hydrogen and hydroxyl; and n is an integer from 0 to 20. In certain cases, n is 10 or less, such as 9, 8, 7, 6, 5, 4, 3, 2, 1 or 0. In certain cases, n is 5. In certain cases, n is 4. In certain cases, n is 3. In certain cases, n is 2. In certain cases, n is 1. In certain cases, n is 0.

In certain embodiments, n is 1, such that the compound of formula (ID) is of the formula (IDa):

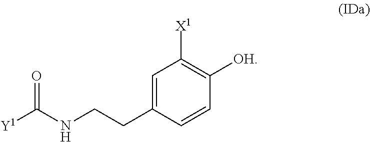

In certain cases of formula (ID) or (IDa), X¹ is hydrogen, such that the biomolecule comprises a phenol moiety. In other embodiments of formula (ID) or (IDa), X¹ is hydroxyl, such that the biomolecule comprises a catechol moiety.

In certain cases, the compound of formula (IDa) is of the formula (IDb):

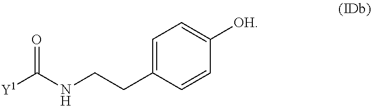

Compounds of any of formula (ID)-(IDb) may be prepared by reacting tyramine, or a corresponding phenol or catechol containing amine, to a biomolecule including a N-hydroxysuccinimide (NHS) ester or maleimide group in a suitable solvent. For example, a compound of formula (IDb) may be prepared by reaction of NHS-ester (Y¹—NHS) with tyramine in dry dimethylformamide (DMF) to provide compound (IDb), as depicted in Scheme 3 below:

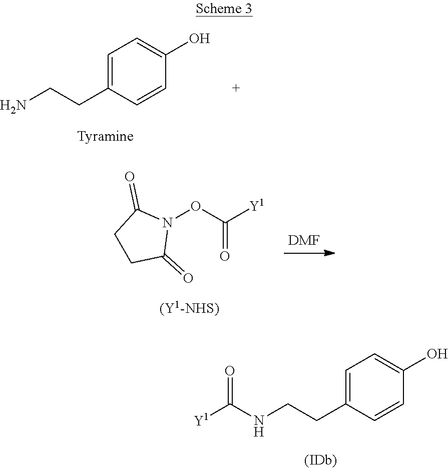

It will be understood that the biomolecule comprising a phenol moiety or a catechol moiety (e.g., of any of formulae (I)—(IDb)) may be prepared by any convenient methods. Many general references providing commonly known chemical synthetic schemes and conditions useful for synthesizing the subject phenol and catechol containing moieties are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978). As disclosed herein, in certain cases the subject phenol moiety is present in a tyrosine residue. The tyrosine residue may be part of the biomolecule of interest. In other cases, the tyrosine moiety may be synthetically introduced into the biomolecule of interest. For example, where the biomolecule is a peptide or a polypeptide, the tyrosine residue may be introduced by standard solid-phase Fmoc peptide chemistry (Fields G B, Noble R L. Solid phase peptide synthesis utilizing 9-fluorenylmethoxycarbonyl amino acids. Int J Pept Protein Res 35: 161-214, 1990). In some cases, a phenol or catechol moiety is part of an unnatural (non-genetically encoded) amino acid that is introduced into a biomolecule of interest. For example, amber codon (TAG) suppression can be used to incorporate a non-genetically encoded amino acid residue that comprises a phenol moiety or a catechol moiety. See, e.g., Chin et al. (2002) J. Am. Chem. Soc. 124:9026; Chin and Schultz (2002) Chem. Biol. Chem. 3:1135; Chin et al. (2002) Proc. Natl. Acad. Sci. USA 99:11020; U.S. 2015/0240249; and US 2018/0171321. As another example, an orthogonal RNA synthetase and/or an orthogonal tRNA can be used for introducing a non-genetically encoded amino acid into a biomolecule, where the non-genetically encoded amino acid comprises a phenol moiety or a catechol moiety.

In some embodiments of any one of formulae (I)—(IDb), the biomolecule of interest comprises one or more groups selected from an active small molecule, an affinity tag, a fluorophore, and a metal-chelating agent. In certain cases, the fluorophore is a rhodamine dye. In certain cases, the fluorophore is a xanthene dye. In certain cases, the fluorophore is Oregon Green 488. In certain cases, the metal-chelating agent is 1,4,7,10-Tetraazacyclododecane-1,4,7,10-tetraacetic acid (also referred to as, DOTA, or tetraxetan). In certain cases, the affinity tag is a biotin moiety (e.g., as described herein).

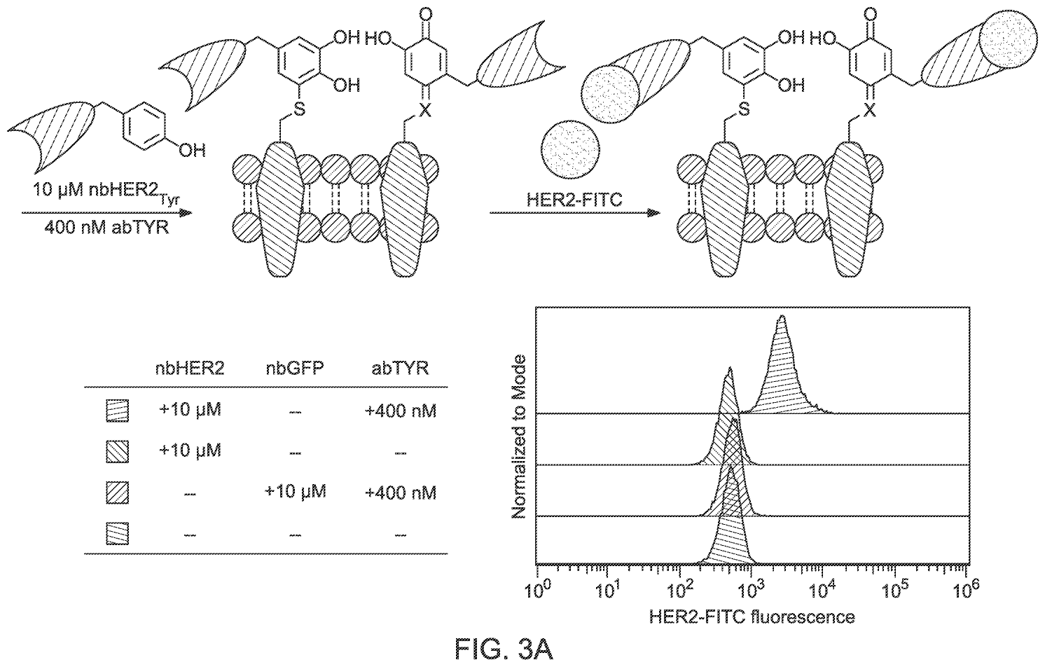

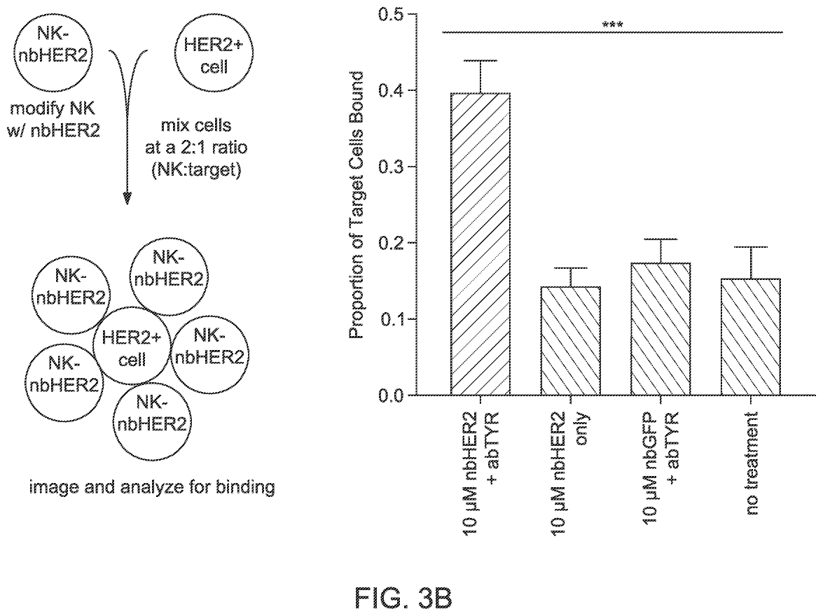

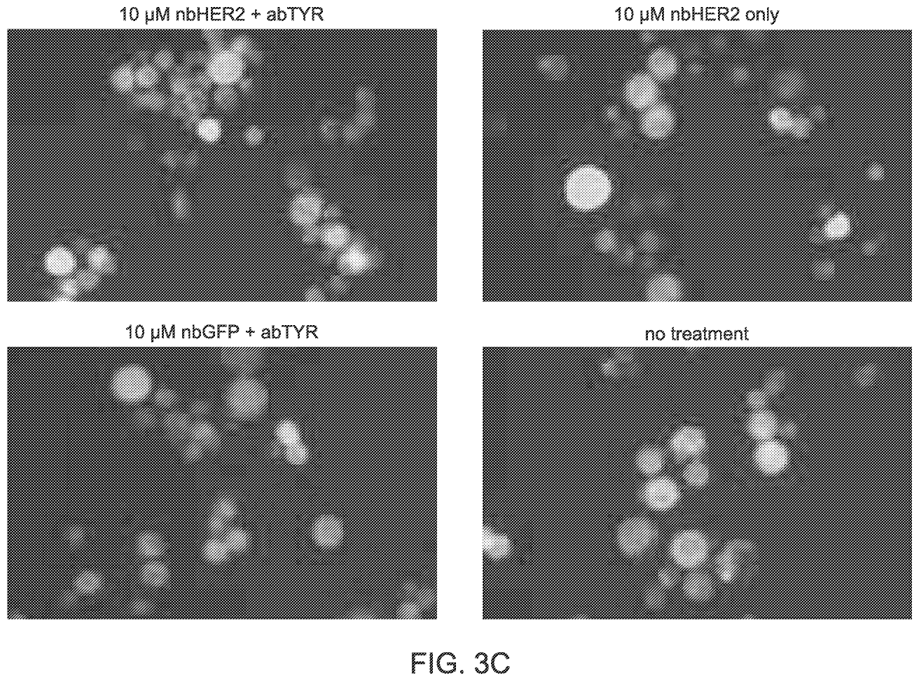

In certain cases, the biomolecule comprising a phenol moiety is described by a structure as depicted in FIG. 3.

### Target Molecule Comprising a Thiol Moiety

Molecules comprising a thiol moiety and suitable for use in the subject methods, as well as methods for producing thiol-comprising molecules suitable for use in the subject methods, are well known in the art.

The target molecules can be naturally occurring, or may be synthetically or recombinantly produced, and may be isolated, substantially purified, or present within the native milieu of the unmodified molecule upon which the thiol-containing target molecule is based (e.g., on a cell surface or within a cell, including within a host animal, e.g., a mammalian animal, such as a murine host (e.g., rat, mouse), hamster, canine, feline, bovine, swine, and the like). In some embodiments, the target molecule is present in vitro in a cell-free reaction. In other embodiments, the target molecule is present in a cell and/or displayed on the surface of a cell. In many embodiments of interest, the target molecule is in a living cell; on the surface of a living cell; in a living organism, e.g., in a living multicellular organism. Suitable living cells include cells that are part of a living multicellular organism; cells isolated from a multicellular organism; immortalized cell lines; and the like.

The target molecule may be composed of D-amino acids, L-amino acids, or both, and may be further modified, either naturally, synthetically, or recombinantly, to include other moieties. For example, the target molecule may be a lipoprotein, a glycoprotein, or other such modified protein.

In general, the target molecule comprises at least one thiol moiety for reaction with a biomolecule comprising a reactive moiety according to the invention, but may comprise 2 or more, 3 or more, 5 or more, 10 or more thiol moieties. The number of thiol moieties that may be present in a target molecule will vary according to the intended application of the modified target molecule of the reaction, the nature of the target molecule itself, and other considerations which will be readily apparent to the ordinarily skilled artisan in practicing the methods as disclosed herein.

The target molecule can be modified to comprise a thiol moiety at the point at which linkage to the biomolecule comprising a reactive moiety is desired. For example, when the target molecule is a peptide or a polypeptide, the target molecule substrate may be modified to contain an N-terminal thiol moiety, thereby producing a subject target peptide or polypeptide comprising a thiol moiety. It will be understood that any convenient location on a peptide or a polypeptide substrate may be modified to contain a thiol moiety and thereby produce a target peptide or polypeptide for use in the subject methods.

In certain embodiments, the target molecule comprising a thiol moiety is a CRISPR-Cas effector polypeptide.

In certain cases, the thiol moiety is present in a cysteine residue. In certain cases, the cysteine residue is native to the CRISPR-Cas effector polypeptide. In other cases, the cysteine residue is introduced into the CRISPR-Cas effector polypeptide. For example, the cysteine residue may be introduced by standard solid-phase Fmoc peptide chemistry (Fields G B, Noble R L. Solid phase peptide synthesis utilizing 9-fluorenylmethoxycarbonyl amino acids. Int J Pept Protein Res 35: 161-214, 1990).

### Modified Target Molecule

In certain embodiments of the subject methods, the modified target molecule produced is of the formula (IV) or (IVA), or a combination thereof. Accordingly, aspects of the disclosure include a compound of formula (IV) or (IVA):

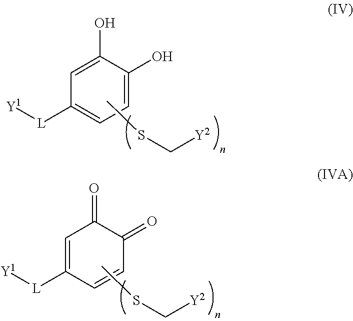

where Y1 is a biomolecule, optionally comprising one or more moieties selected from, an active small molecule, an affinity tag, a fluorophore, and a metal-chelating agent; L is an optional linker; Y2 is a second biomolecule; and n is an integer from 1 to 3.

In certain embodiments of formula (IV) or (IVA), n is less than 3, such as 2 or 1. In certain cases, n is 2. In certain cases, n is 1. In certain cases, the subject modified target molecule is a compound of formula (IV). In certain cases, the subject modified target molecule is a compound of formula (IVA).

In some embodiments, the modified target molecule of formula (IV), n is 1 and the compounds is described by any of formulae (IV1)-(IV3):

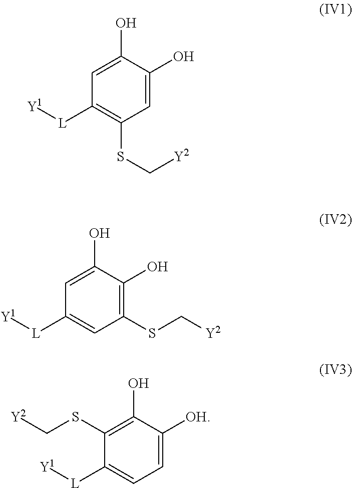

In some embodiments, the modified target molecule of formula (IV), n is 2 and the compounds is described by any of formulae (IV4)-(IV5):

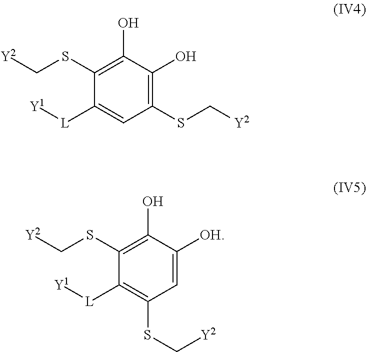

In some embodiments, the modified target molecule is of formula (IVA), n is 1, and the compound is described by any of formulae (IVA1)-(IVA3):

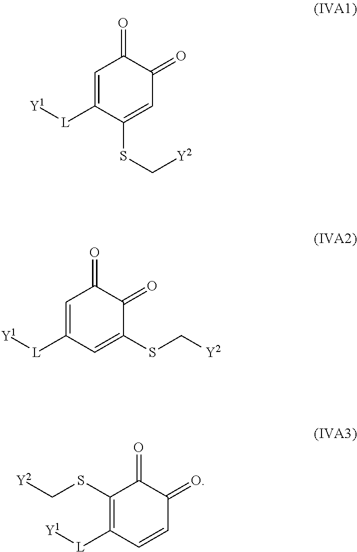

In some embodiments, the modified target molecule is of formula (IVA), n is 2, and the compound is described by any of formulae (IVA4)-(IVA5):

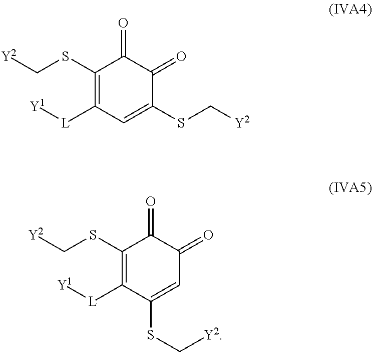

In certain embodiments, the modified target molecule includes a linker (e.g., as described herein). Suitable linkers include, but are not limited to, a carboxylic acid, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone, a halosulfonyl, a nitrile, a nitro, and a peptide linker.

Exemplary linkers for use in linking the orthoquinone to the biomolecule (Y′) will in some embodiments include an amide, such as —(CR¹)NHC(O)—, wherein R¹ is selected from hydrogen, or a substituent (e.g., as described herein) and m is an integer from 1 to 20. Exemplary linkers may also include a PEG or a substituted PEG linker, e.g., as described herein.

In certain embodiments, the linker is a cleavable linker, e.g., as described herein.

In certain embodiments, the modified target molecule is described by the formula (LVB) or (IVC):

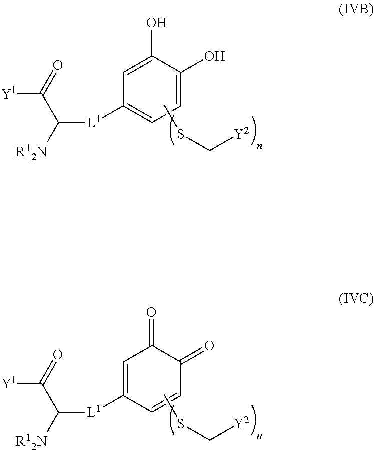

where Y¹ is a biomolecule optionally comprising one or more groups selected from, an active small molecule, an affinity tag, a fluorophore, and a metal-chelating agent; each R¹ is independently selected from hydrogen, acyl, substituted acyl, alkyl, and substituted alkyl; Y² is a second biomolecule; L¹ is a linker selected from a straight or branched alkyl, a straight or branched substituted alkyl, a polyethylene glycol (PEG), a substituted PEG, and one or more peptides; and n is an integer from 1 to 3.

In certain embodiments of formula (IVB) or (IVC), n is less than 3, such as 2 or 1. In certain cases, n is 2. In certain cases, n is 1. In certain cases, the subject modified target molecule is a compound of formula (IVB). In certain cases, the subject modified target molecule is a compound of formula (IVC).

In some embodiments, the modified target molecule of formula (IVB), n is 1 and the compounds is described by any of formulae (IVB1)-(IVB3):

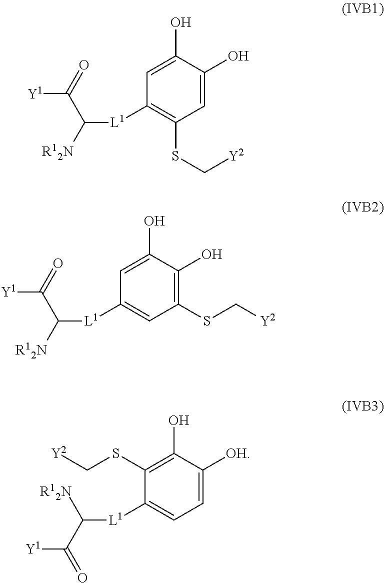

In some embodiments, the modified target molecule of formula (IVB), n is 2 and the compounds is described by any of formulae (IVB4)-(IVB5):

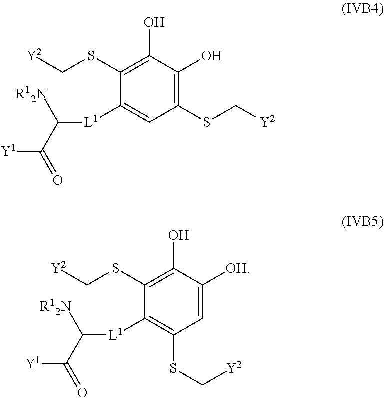

In some embodiments, the modified target molecule is of formula (IVC), n is 1, and the compound is described by any of formulae (IVC1)-(IVC3):

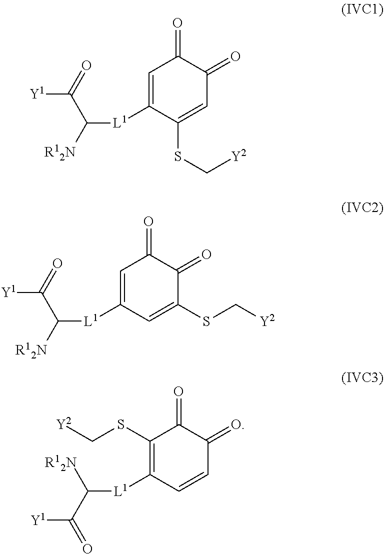

In some embodiments, the modified target molecule is of formula (IVC), n is 2, and the compound is described by any of formulae (IVC4)-(IVC5):

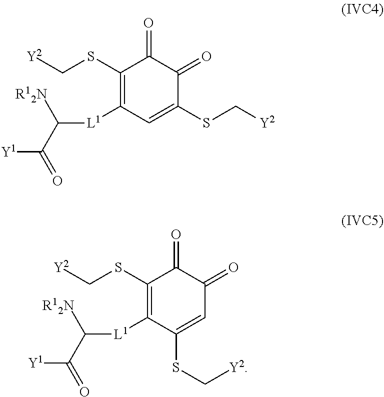

In certain embodiments of any of formulae (IVB)—(IVC5), at least one R¹ is hydrogen. In certain cases, both R¹ groups are hydrogen. In certain cases, one R group is hydrogen, and the other R¹ group is selected from alkyl, substituted alkyl, acyl and substituted acyl. In certain cases, one R¹ group is hydrogen and the other R¹ group is alkyl. In some cases, one R¹ group is hydrogen and the other R¹ group is substituted alkyl. In some cases, one R¹ group is hydrogen and the other R¹ group is acyl. In some cases, one R¹ group is hydrogen, and the other R¹ group is substituted acyl. In some cases the acyl group is of the formula —C(O)R⁴, wherein R⁴ is a lower alkyl group, e.g., methyl, ethyl, propyl, butyl, pentyl, or hexyl. In some cases, the substituted acyl group is of the formula —C(O)R⁴NH₂, wherein R⁴ is a lower alkyl group, e.g., methyl, ethyl, propyl, butyl, pentyl or hexyl. In some cases, the substituted acyl group is of the formula —C(O)CH₂NH₂.

In certain embodiments of any of formulae (IVB)—(IVC5), L¹ is a straight or branched alkyl. In certain cases, L¹ is a lower alkyl group, e.g., methyl, ethyl, propyl, butyl, pentyl, or hexyl. In certain cases, L¹ is a substituted alkyl group. In certain cases, L¹ is a substituted lower alkyl group. In certain cases, L¹ is a PEG or substituted PEG (e.g., as described herein). In certain other cases, L¹ is a peptide. In certain other cases, L¹ is a polypeptide. In certain cases, L¹ is a linear linker of 1-12 atoms in length, such as 1-10, 1-8 or 1-6 atoms in length, e.g., 1, 2, 3, 4, 5 or 6 atoms in length. The linker L¹ can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl). In certain cases, the linker L¹ can include a keto (C═O) group. In certain cases, the keto group together with an amino, thiol or ether group in the linker chain can provide an amido, an ester or thioester group linkage.

In certain embodiments, the linking group L¹ is a cleavable linker, e.g., as described herein.

In certain embodiments, the modified target molecule is described by any of the formulae (IVD)-(IVG):

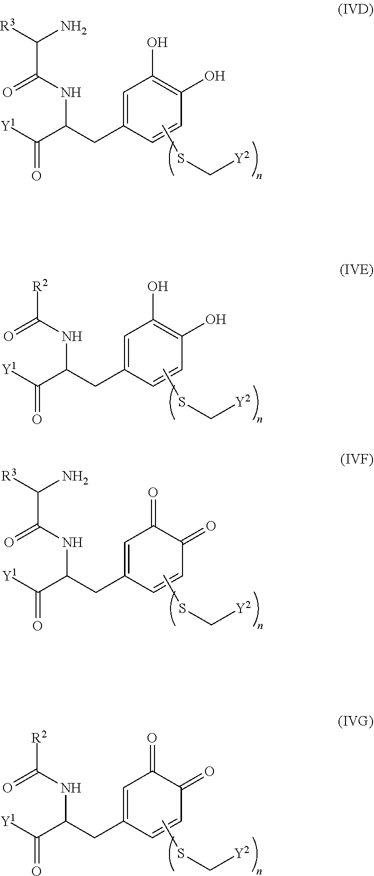

where R² is selected from alkyl, and substituted alkyl; R³ is selected from, hydrogen, alkyl substituted alkyl, a peptide, and a polypeptide; and n is an integer from 1 to 3.

In certain embodiments of any of formulas (IVD)-(IVG), n is less than 3, such as 2 or 1. In certain cases, n is 2. In certain cases, n is 1. In certain cases, the subject modified target molecule is a compound of formula (IVD). In certain cases, the subject modified target molecule is a compound of formula (IVE). In certain cases, the subject modified target molecule is a compound of formula (IVF). In certain cases, the subject modified target molecule is a compound of formula (IVG).

In certain embodiments, any of formulae (IVD)-(IVG) may have relative stereochemistry as shown in the following structures:

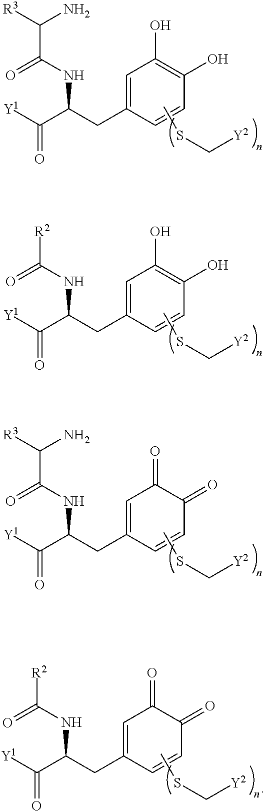

In some embodiments, the modified target molecule of formula (IVD), n is 1 and the compounds is described by any of formulae (IVD1)-(IVD3):

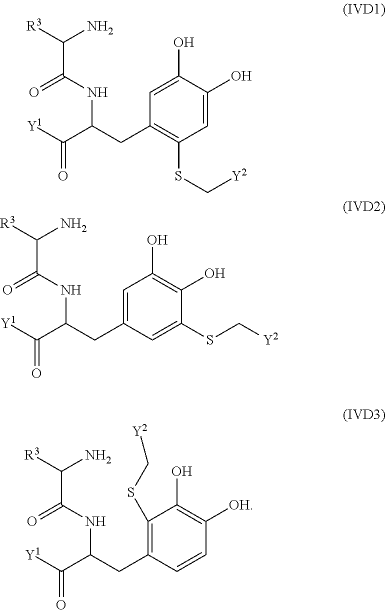

In some embodiments, the modified target molecule of formula (IVD), n is 2 and the compounds is described by any of formulae (IVD4)-(IVD5):

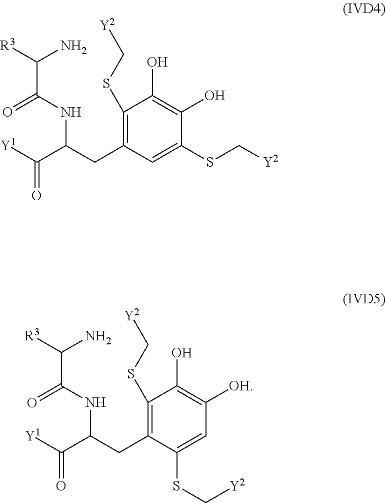

In some embodiments, the modified target molecule of formula (IVE), n is 1 and the compounds is described by any of formulae (IVE1)-(IVE3):

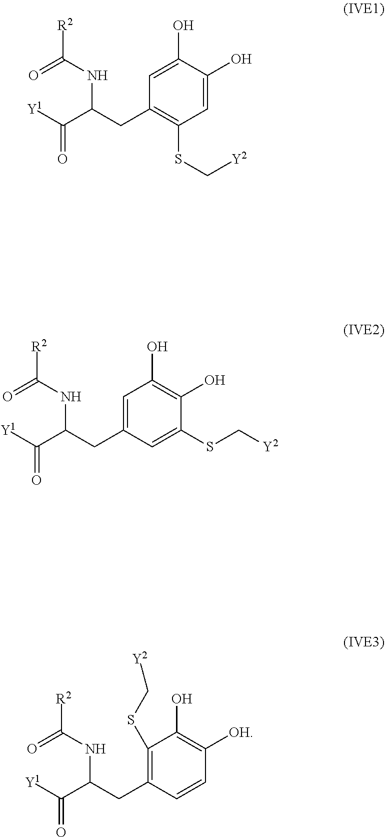

In some embodiments, the modified target molecule of formula (IVE), n is 2 and the compounds is described by any of formulae (IVE4)-(IVE5):

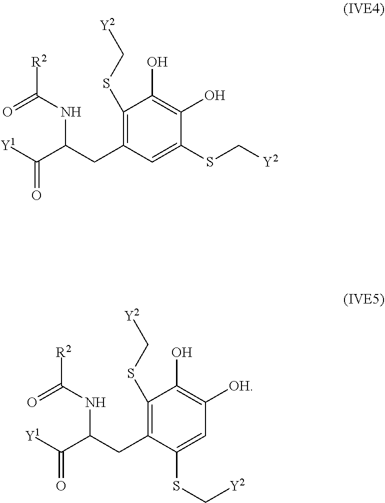

In some embodiments, the modified target molecule of formula (IVF), n is 1 and the compounds is described by any of formulae (IVF1)-(IVF3):

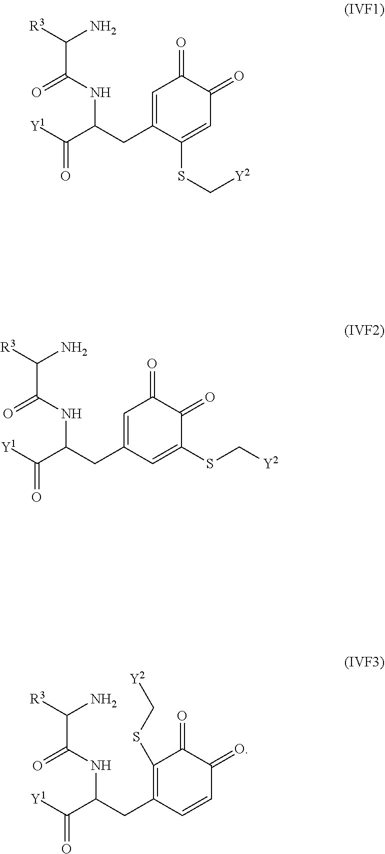

In some embodiments, the modified target molecule of formula (IVF), n is 2 and the compounds is described by any of formulae (IVF4)-(IVF5):

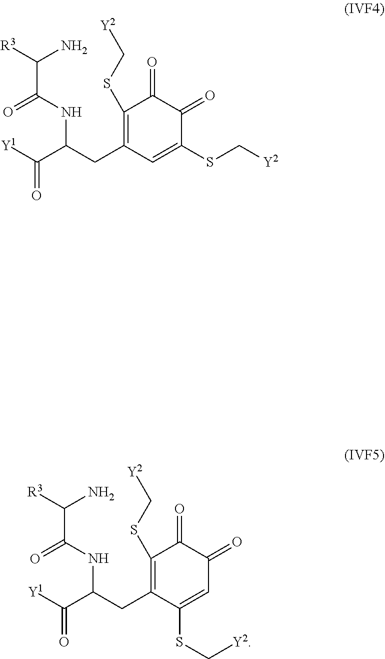

In some embodiments, the modified target molecule of formula (IVG), n is 1 and the compounds is described by any of formulae (IVG1)-(IVG3):

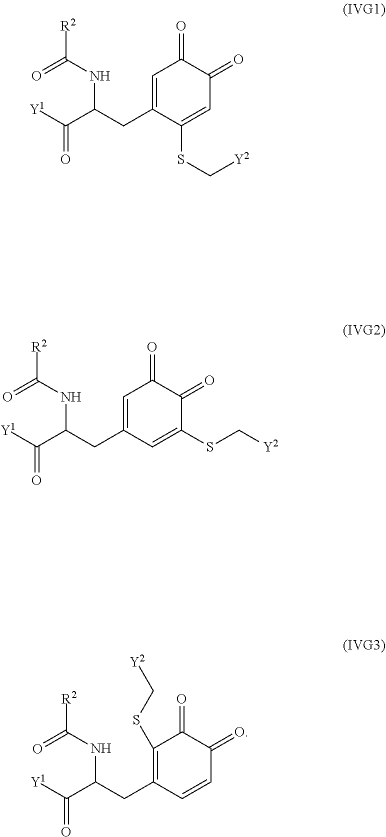

In some embodiments, the modified target molecule of formula (IVG), n is 2 and the compounds is described by any of formulae (IVG4)-(IVG5):

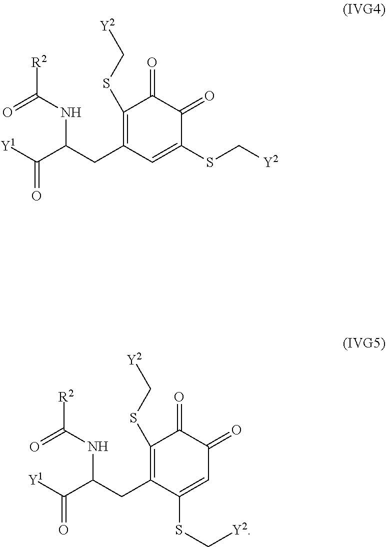

In certain embodiments of the target molecules described herein, R² is an alkyl group. In certain cases, R² is a substituted alkyl group. In certain cases, the alkyl group is a lower alkyl group, e.g., methyl, ethyl, propyl, butyl, pentyl or hexyl.

In certain embodiments of the target molecules described herein, R³ is hydrogen. In certain cases, R³ an alkyl group. In certain cases, R³ is a substituted alkyl group. In certain cases the alkyl group is a lower alkyl group, e.g., methyl, ethyl, propyl, butyl, pentyl or hexyl. In certain cases, R³ is a peptide. In certain cases, R³ is a polypeptide.

In certain embodiments, the modified target molecule is described by the formula (IVH)

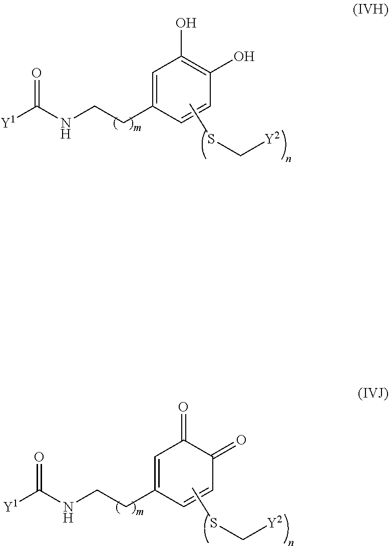

where Y¹ is a biomolecule optionally comprising one or more groups selected from, an active small molecule, an affinity tag, a fluorophore, and a metal-chelating agent; Y² is a second biomolecule; n is an integer from 1 to 3; and m is an integer from 0 to 20. In certain cases, m is 10 or less, such as 9, 8, 7, 6, 5, 4, 3, 2, 1 or 0. In certain cases, m is 5. In certain cases, m is 4. In certain cases, m is 3. In certain cases, m is 2. In certain cases, m is 1. In certain cases, m is 0.

In certain embodiments of the subject methods, the modified target molecule is described by the formula (IVK) or IVL):

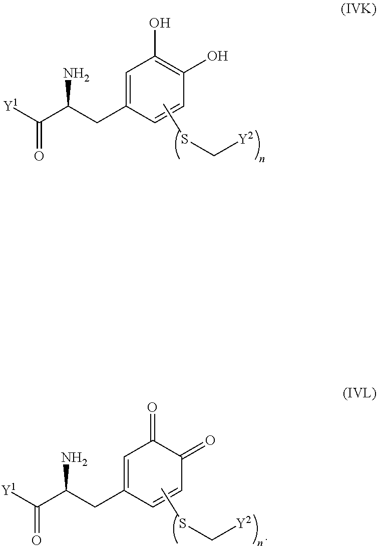

In certain embodiments of any of formulae (IVH)—(IVJ), n is less than 3, such as 2 or 1. In certain cases, n is 2. In certain cases, n is 1.

In some embodiments, the modified target molecule of formula (IVH), n is 1 and the compounds is described by any of formulae (IVH1)-(IVH3):

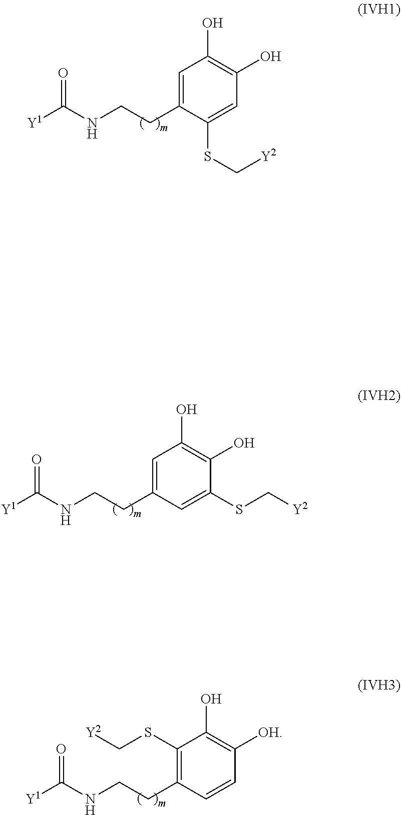

In some embodiments, the modified target molecule of formula (IVH), n is 2 and the compounds is described by any of formulae (IVH4)-(IVH5):

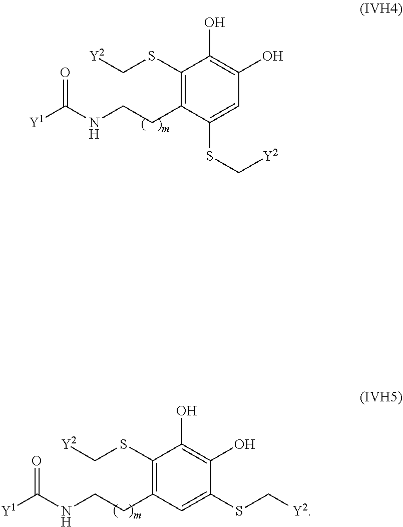

In some embodiments, the modified target molecule of formula (IVJ), n is 1 and the compounds is described by any of formulae (IVJ1)-(IVJ3):

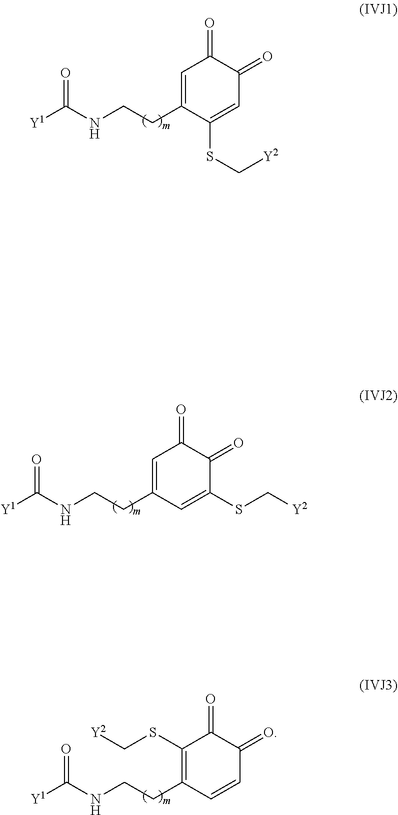

In some embodiments, the modified target molecule of formula (IVJ), n is 2 and the compounds is described by any of formulae (IVJ4)-(IVJ5):

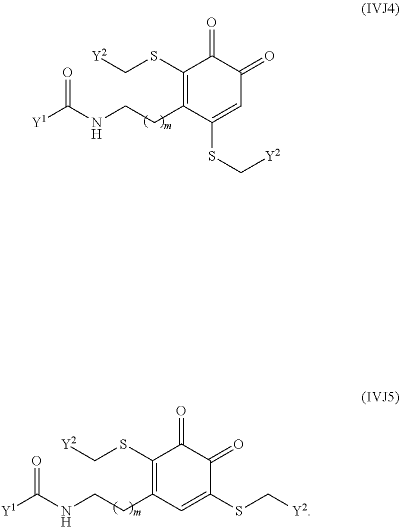

In certain embodiments, the target molecule comprising a thiol group is a CRISPR-Cas effector polypeptide (e.g., as described herein).

In certain embodiments of any one of formulae (V) to (VJ5), Y¹ is a polypeptide. In certain cases, the Y¹ polypeptide is selected from a fluorescent protein, an antibody, and an enzyme. In certain cases, the fluorescent protein is a green fluorescent protein. Other suitable polypeptides are described elsewhere herein.

In certain embodiments of the subject methods, the modified target molecule produced is of the formula (V) or (VA), or a combination thereof. Accordingly, aspects of the disclosure include a compound of formula (V) or (VA):

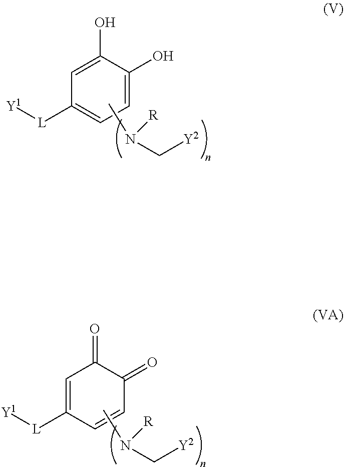

where Y1 is a biomolecule, optionally comprising one or more moieties selected from, an active small molecule, an affinity tag, a fluorophore, and a metal-chelating agent; L is an optional linker; Y2 is a second biomolecule; R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl and n is an integer from 1 to 3.

In certain embodiments of formula (V) or (VA), n is less than 3, such as 2 or 1. In certain cases, n is 2. In certain cases, n is 1. In certain cases, the subject modified target molecule is a compound of formula (V). In certain cases, the subject modified target molecule is a compound of formula (VA).

In some embodiments, the modified target molecule of formula (V), n is 1 and the compounds is described by any of formulae (V1)-(V3):

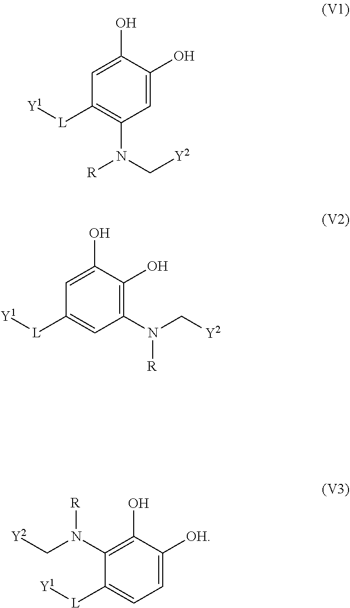

In some embodiments, the modified target molecule of formula (IV), n is 2 and the compounds is described by any of formulae (V4)-(V5):

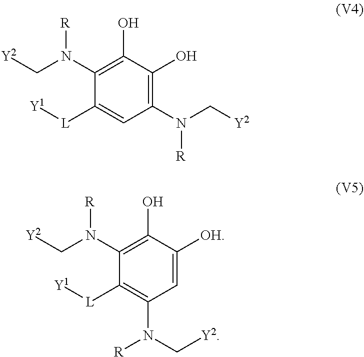

In some embodiments, the modified target molecule is of formula (VA), n is 1, and the compound is described by any of formulae (VA1)-(VA3):

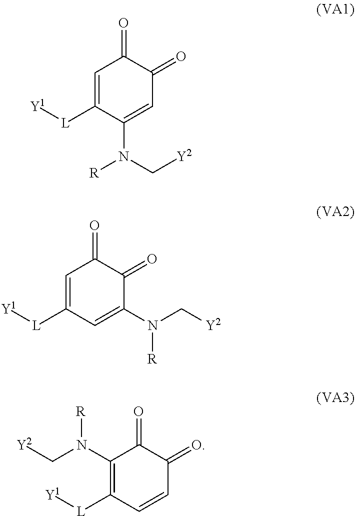

In some embodiments, the modified target molecule is of formula (VA), n is 2, and the compound is described by any of formulae (VA4)-(VA5):

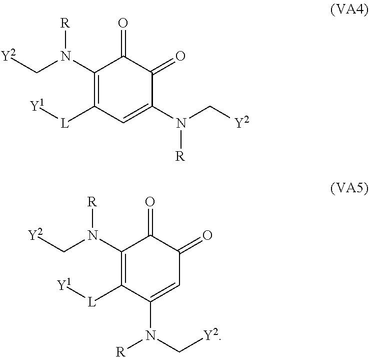

In certain embodiments, the modified target molecule includes a linker (e.g., as described herein). Suitable linkers include, but are not limited to, a carboxylic acid, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone, a halosulfonyl, a nitrile, a nitro, and a peptide linker.

Exemplary linkers for use in linking the orthoquinone to the biomolecule (Y′) will in some embodiments include an amide, such as —(CR¹₂)ₘNHC(O)—, wherein R¹ is selected from hydrogen, or a substituent (e.g., as described herein) and m is an integer from 1 to 20. Exemplary linkers may also include a PEG or a substituted PEG linker, e.g., as described herein.

In certain embodiments, the linker is a cleavable linker, e.g., as described herein.

In certain embodiments, the modified target molecule is described by the formula (VB) or (VC):

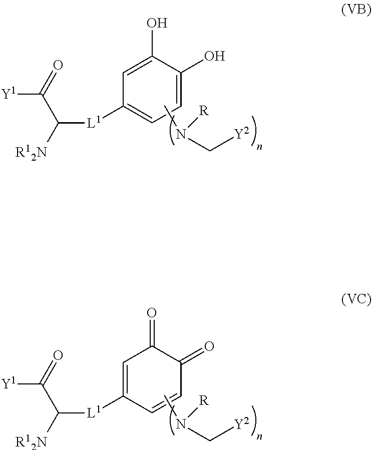

where Y¹ is a biomolecule optionally comprising one or more groups selected from, an active small molecule, an affinity tag, a fluorophore, and a metal-chelating agent; R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; each R¹ is independently selected from hydrogen, acyl, substituted acyl, alkyl, and substituted alkyl; Y² is a second biomolecule; L¹ is a linker selected from a straight or branched alkyl, a straight or branched substituted alkyl, a polyethylene glycol (PEG), a substituted PEG, and one or more peptides; and n is an integer from 1 to 3.

In certain embodiments of formula (VB) or (VC), n is less than 3, such as 2 or 1. In certain cases, n is 2. In certain cases, n is 1. In certain cases, the subject modified target molecule is a compound of formula (VB). In certain cases, the subject modified target molecule is a compound of formula (VC).

In some embodiments, the modified target molecule of formula (VB), n is 1 and the compounds is described by any of formulae (VB1)-(VB3):

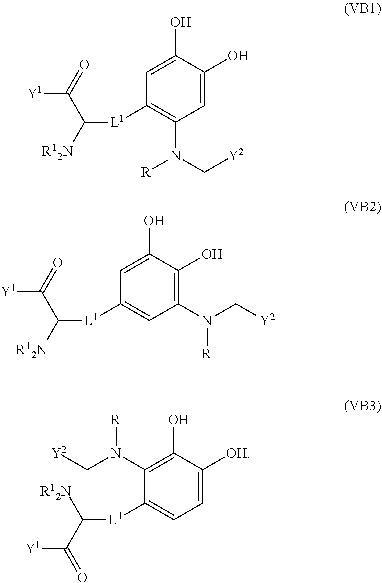

In some embodiments, the modified target molecule of formula (VB), n is 2 and the compounds is described by any of formulae (VB4)-(VB5):

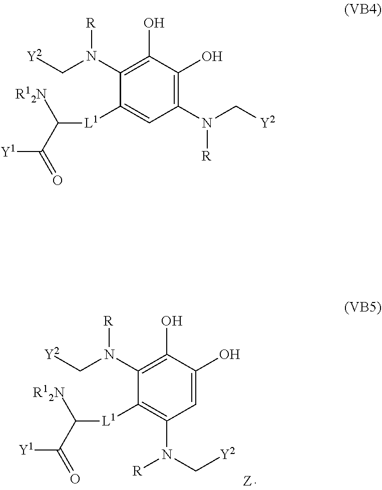

In some embodiments, the modified target molecule is of formula (VC), n is 1, and the compound is described by any of formulae (VC1)-(VC3):

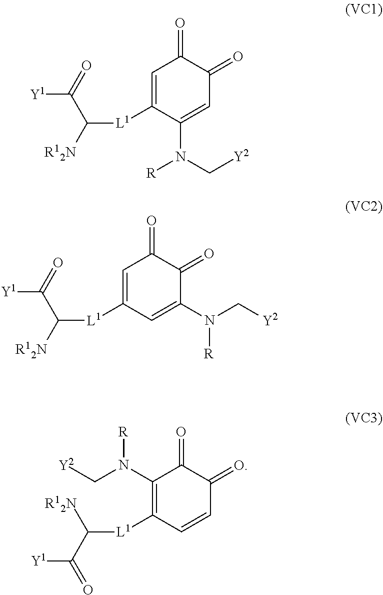

In some embodiments, the modified target molecule of formula (VB), n is 2 and the compounds is described by any of formulae (VB4)-(VB5):

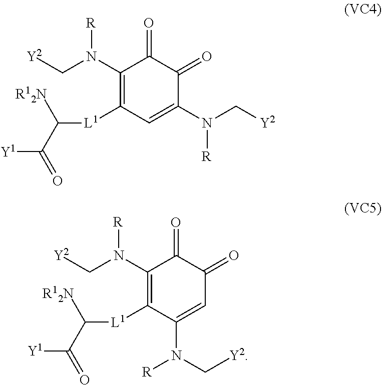

In certain embodiments of any of formulae (VB)—(VC5), at least one R¹ is hydrogen. In certain cases, both R¹ groups are hydrogen. In certain cases, one R group is hydrogen, and the other R¹ group is selected from alkyl, substituted alkyl, acyl and substituted acyl. In certain cases, one R¹ group is hydrogen and the other R¹ group is alkyl. In some cases, one R¹ group is hydrogen and the other R¹ group is substituted alkyl. In some cases, one R¹ group is hydrogen and the other R¹ group is acyl. In some cases, one R¹ group is hydrogen, and the other R¹ group is substituted acyl. In some cases the acyl group is of the formula —C(O)R⁴, wherein R⁴ is a lower alkyl group, e.g., methyl, ethyl, propyl, butyl, pentyl, or hexyl. In some cases, the substituted acyl group is of the formula —C(O)R⁴NH₂, wherein R⁴ is a lower alkyl group, e.g., methyl, ethyl, propyl, butyl, pentyl or hexyl. In some cases, the substituted acyl group is of the formula —C(O)CH₂NH₂.

In certain embodiments of any of formulae (VB)—(VC5), L¹ is a straight or branched alkyl. In certain cases, L¹ is a lower alkyl group, e.g., methyl, ethyl, propyl, butyl, pentyl, or hexyl. In certain cases, L¹ is a substituted alkyl group. In certain cases, L¹ is a substituted lower alkyl group. In certain cases, L¹ is a PEG or substituted PEG (e.g., as described herein). In certain other cases, L¹ is a peptide. In certain other cases, L¹ is a polypeptide. In certain cases, L¹ is a linear linker of 1-12 atoms in length, such as 1-10, 1-8 or 1-6 atoms in length, e.g., 1, 2, 3, 4, 5 or 6 atoms in length. The linker L¹ can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl). In certain cases, the linker L¹ can include a keto (C═O) group. In certain cases, the keto group together with an amino, thiol or ether group in the linker chain can provide an amido, an ester or thioester group linkage.

In certain embodiments, the linking group L¹ is a cleavable linker, e.g., as described herein.

In certain embodiments, the modified target molecule is described by any of the formulae (VD)-(VG):

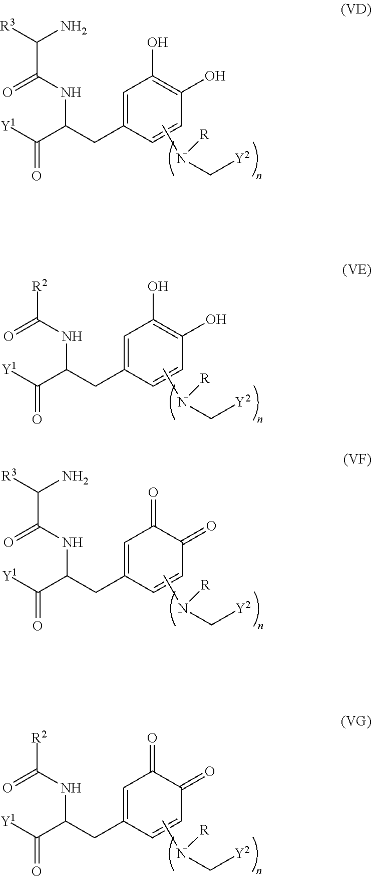

where R² is selected from alkyl, and substituted alkyl; R³ is selected from, hydrogen, alkyl substituted alkyl, a peptide, and a polypeptide; and n is an integer from 1 to 3.

In certain embodiments of any of formulas (VD)-(VG), n is less than 3, such as 2 or 1. In certain cases, n is 2. In certain cases, n is 1. In certain cases, the subject modified target molecule is a compound of formula (VD). In certain cases, the subject modified target molecule is a compound of formula (VE). In certain cases, the subject modified target molecule is a compound of formula (VF). In certain cases, the subject modified target molecule is a compound of formula (VG).

In certain embodiments, any of formulae (VD)-(VG) may have relative stereochemistry as shown in the following structures:

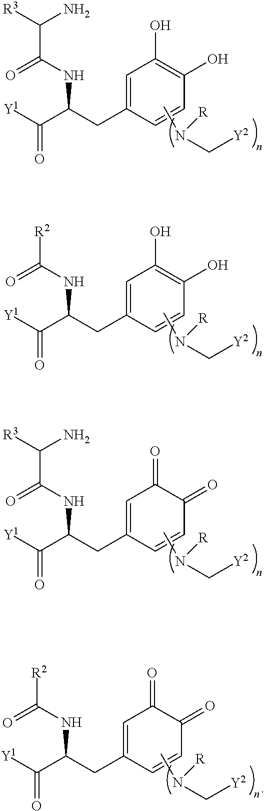

In some embodiments, the modified target molecule of formula (VD), n is 1 and the compounds is described by any of formulae (VD1)-(VD3):

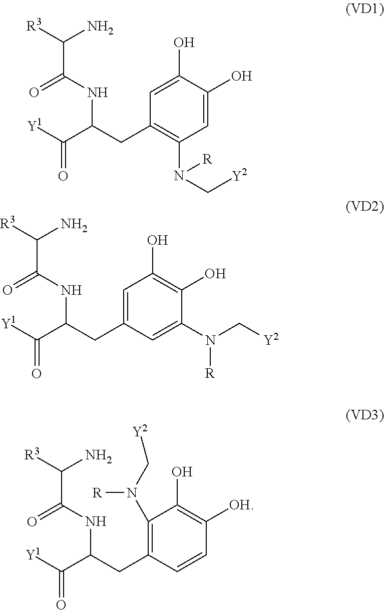

In some embodiments, the modified target molecule of formula (VD), n is 2 and the compounds is described by any of formulae (VD4)-(VD5):

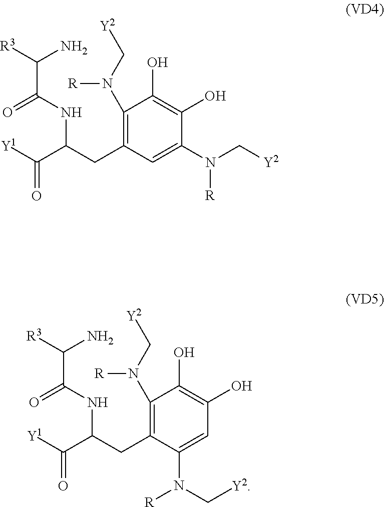

In some embodiments, the modified target molecule of formula (VE), n is 1 and the compounds is described by any of formulae (VE1)-(VE3):

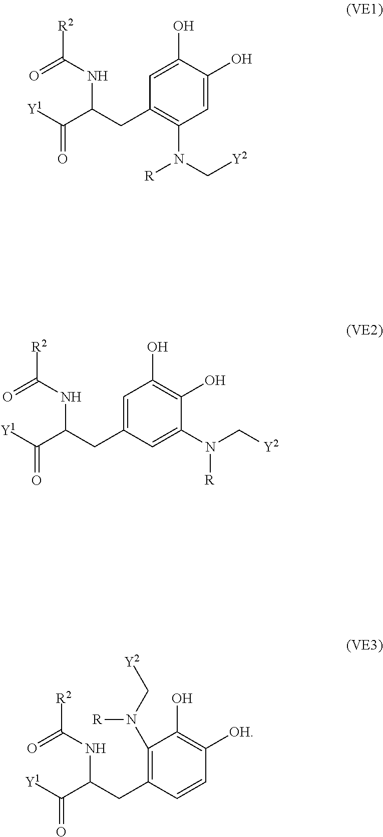

In some embodiments, the modified target molecule of formula (VE), n is 2 and the compounds is described by any of formulae (VE4)-(VE5):

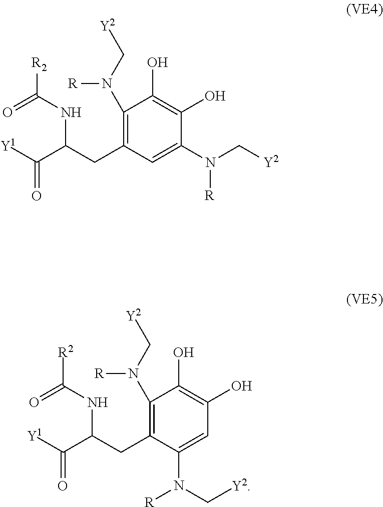

In some embodiments, the modified target molecule of formula (VF), n is 1 and the compounds is described by any of formulae (VF1)-(VF3):

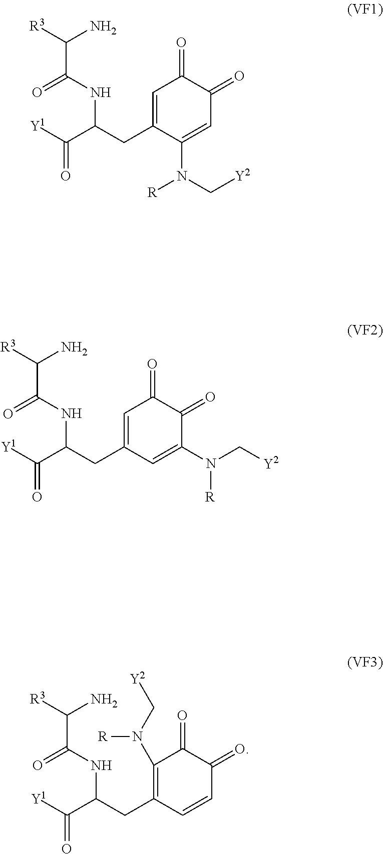

In some embodiments, the modified target molecule of formula (VF), n is 2 and the compounds is described by any of formulae (VF4)-(VF5):

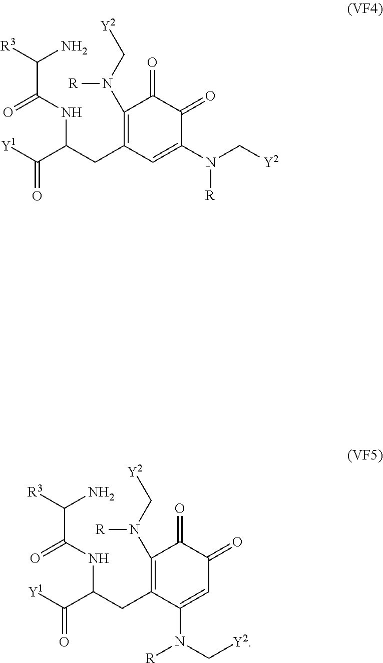

In some embodiments, the modified target molecule of formula (VG), n is 1 and the compounds is described by any of formulae (VG1)-(VG3):

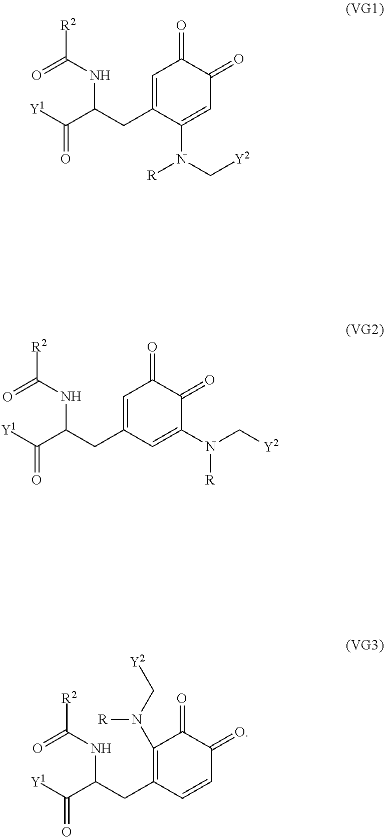

In some embodiments, the modified target molecule of formula (VG), n is 2 and the compounds is described by any of formulae (VG4)-(VG5):

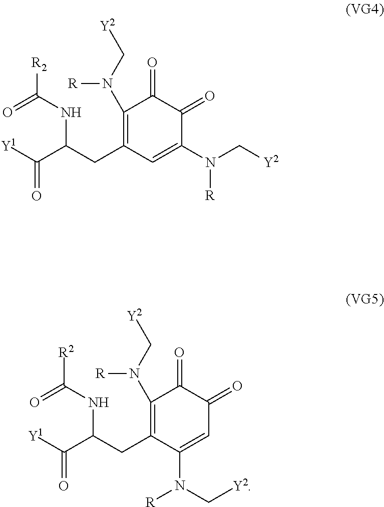

In certain embodiments of the target molecules described herein, R² is an alkyl group. In certain cases, R² is a substituted alkyl group. In certain cases, the alkyl group is a lower alkyl group, e.g., methyl, ethyl, propyl, butyl, pentyl or hexyl.

In certain embodiments of the target molecules described herein, R³ is hydrogen. In certain cases, R³ an alkyl group. In certain cases, R³ is a substituted alkyl group. In certain cases the alkyl group is a lower alkyl group, e.g., methyl, ethyl, propyl, butyl, pentyl or hexyl. In certain cases, R³ is a peptide. In certain cases, R³ is a polypeptide.

In certain embodiments, the modified target molecule is described by the formula (VH) or (VJ):

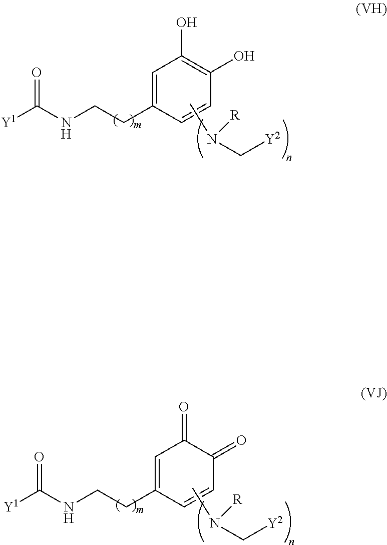

where Y¹ is a biomolecule optionally comprising one or more groups selected from, an active small molecule, an affinity tag, a fluorophore, and a metal-chelating agent; Y² is a second biomolecule; n is an integer from 1 to 3; and m is an integer from 0 to 20. In certain cases, m is 10 or less, such as 9, 8, 7, 6, 5, 4, 3, 2, 1 or 0. In certain cases, m is 5. In certain cases, m is 4. In certain cases, m is 3. In certain cases, m is 2. In certain cases, m is 1. In certain cases, m is 0.

In certain embodiments of the subject methods, the modified target molecule is described by the formula (VK) or VL):

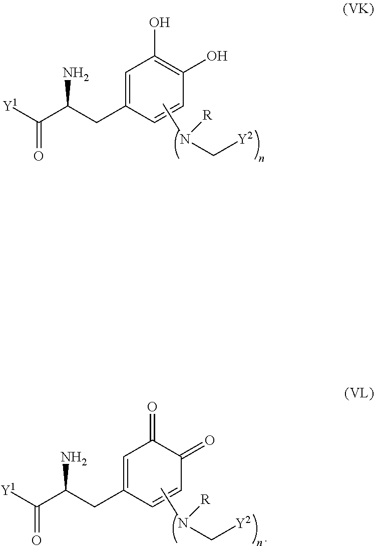

In certain embodiments of any of formulae (VH)—(VJ), n is less than 3, such as 2 or 1. In certain cases, n is 2. In certain cases, n is 1.

In some embodiments, the modified target molecule of formula (VH), n is 1 and the compounds is described by any of formulae (VH1)-(VH3):

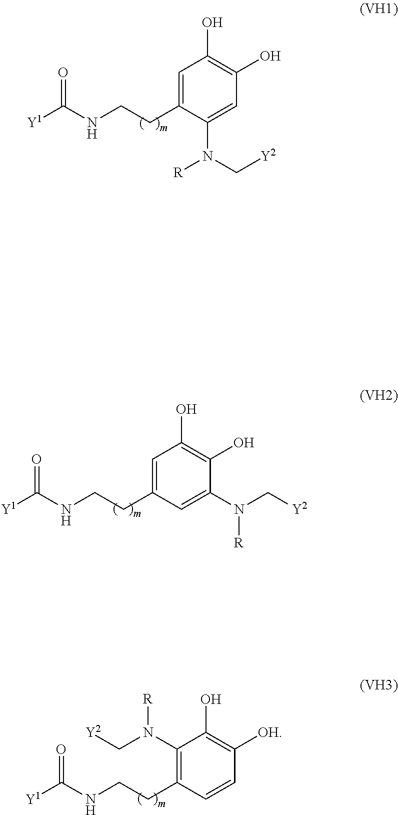

In some embodiments, the modified target molecule of formula (IVH), n is 2 and the compounds is described by any of formulae (VH4)-(VH5):

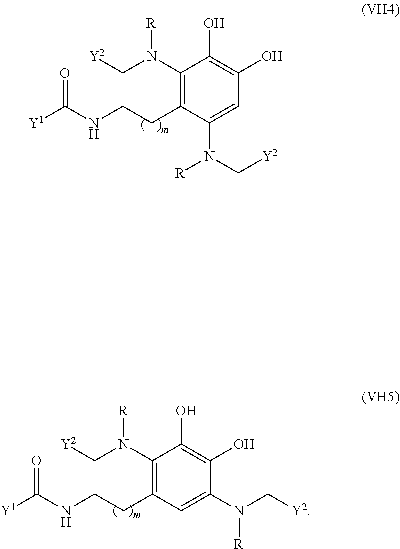

In some embodiments, the modified target molecule of formula (VJ), n is 1 and the compounds is described by any of formulae (VJ1)-(VJ3):

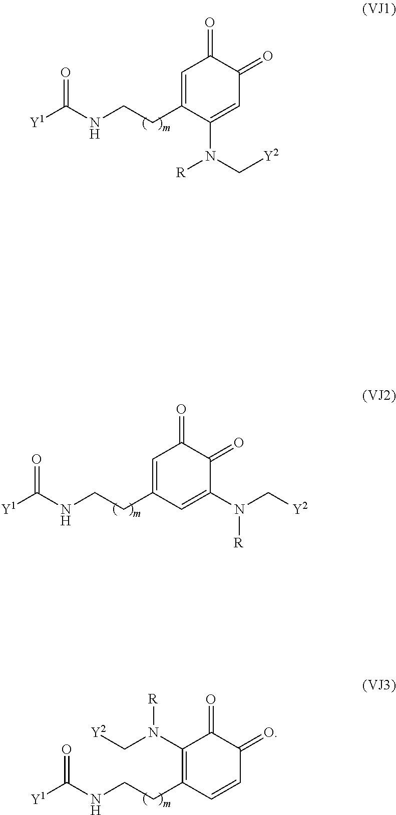

In some embodiments, the modified target molecule of formula (IVH), n is 2 and the compounds is described by any of formulae (VH4)-(VH5):

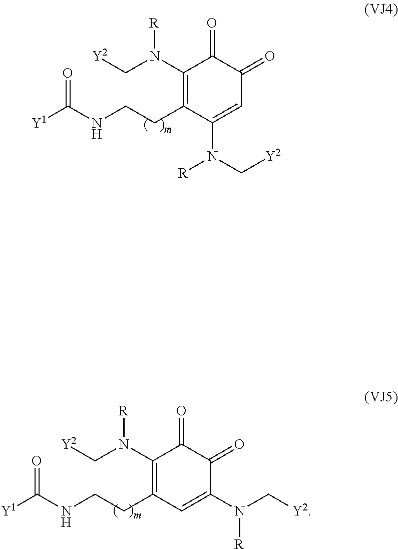

In certain embodiments, the target molecule comprising a thiol group is a CRISPR-Cas effector polypeptide (e.g., as described herein).

In certain embodiments of any one of formulae (V) to (VJ5), Y¹ is a polypeptide. In certain cases, the Y¹ polypeptide is selected from a fluorescent protein, an antibody, and an enzyme. In certain cases, the fluorescent protein is a green fluorescent protein. Other suitable polypeptides are described elsewhere herein.

In some embodiments of any one of formulae (V) to (VJ5), R is hydrogen.

In certain embodiments of any one of formulae (VI) to (VIJ5), Y¹ is a polypeptide. In certain cases, the Y¹ polypeptide is selected from a fluorescent protein, an antibody, and an enzyme. In certain cases, the fluorescent protein is a green fluorescent protein. Other suitable polypeptides are described elsewhere herein.

In certain embodiments of the subject methods, the modified target molecule produced is of the formula (VI) or (VIA), or a combination thereof. Accordingly, aspects of the disclosure include a compound of formula (VI) or (VIA):

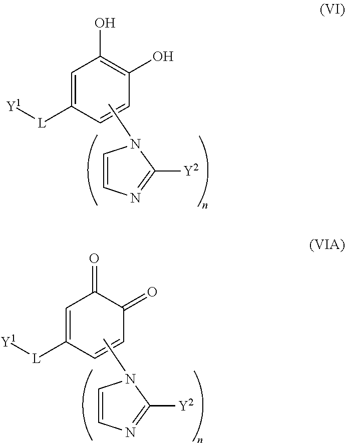

where Y1 is a biomolecule, optionally comprising one or more moieties selected from, an active small molecule, an affinity tag, a fluorophore, and a metal-chelating agent; L is an optional linker; Y2 is a second biomolecule; and n is an integer from 1 to 3.

In certain embodiments of formula (VI) or (VIA), n is less than 3, such as 2 or 1. In certain cases, n is 2. In certain cases, n is 1. In certain cases, the subject modified target molecule is a compound of formula (VI). In certain cases, the subject modified target molecule is a compound of formula (VIA).

In some embodiments, the modified target molecule of formula (VI), n is 1 and the compounds is described by any of formulae (V1)-(V3):

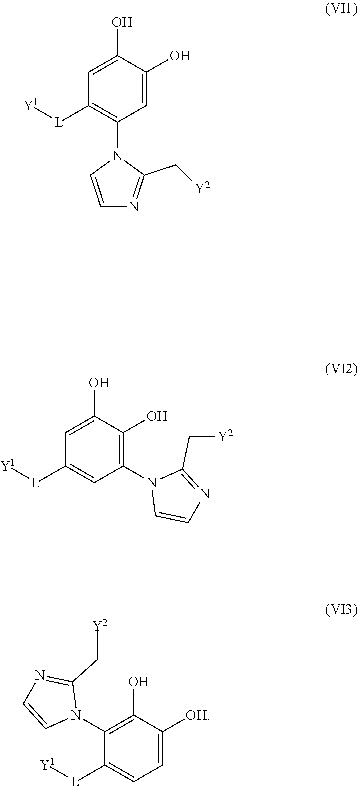

In some embodiments, the modified target molecule of formula (IV), n is 2 and the compounds is described by any of formulae (V14)-(VI5):

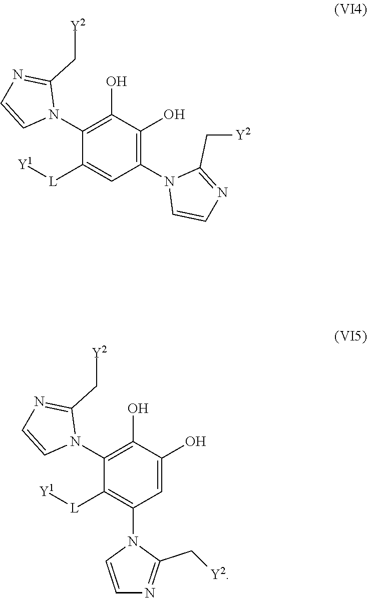

In some embodiments, the modified target molecule is of formula (VA), n is 1, and the compound is described by any of formulae (VIA1)-(VIA3):

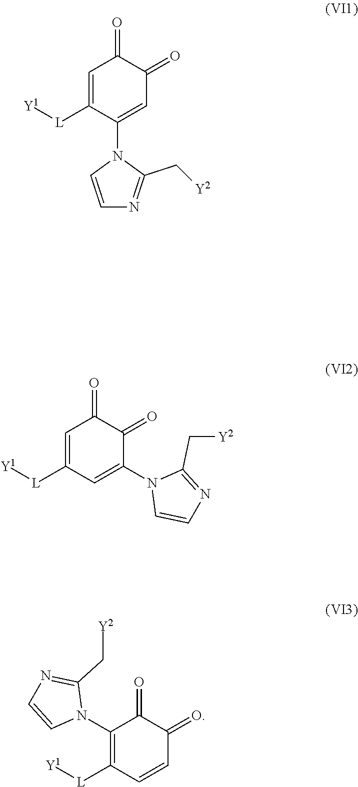

In some embodiments, the modified target molecule is of formula (VIA), n is 2, and the compound is described by any of formulae (VIA4)-(YA5):

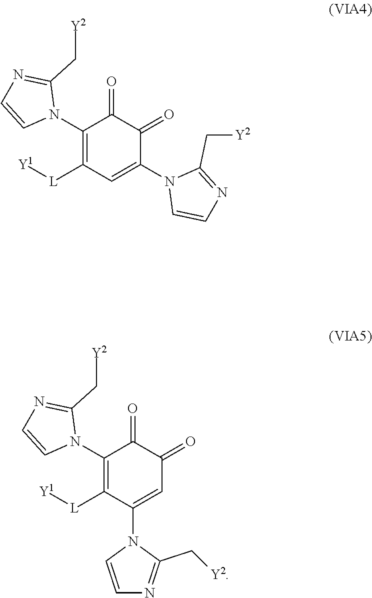

In certain embodiments, the modified target molecule includes a linker (e.g., as described herein). Suitable linkers include, but are not limited to, a carboxylic acid, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone, a halosulfonyl, a nitrile, a nitro, and a peptide linker.

Exemplary linkers for use in linking the orthoquinone to the biomolecule (Y′) will in some embodiments include an amide, such as —(CR¹₂)ₘNHC(O)—, wherein R¹ is selected from hydrogen, or a substituent (e.g., as described herein) and m is an integer from 1 to 20. Exemplary linkers may also include a PEG or a substituted PEG linker, e.g., as described herein.

In certain embodiments, the linker is a cleavable linker, e.g., as described herein.

In certain embodiments, the modified target molecule is described by the formula (VIB) or (VIC):

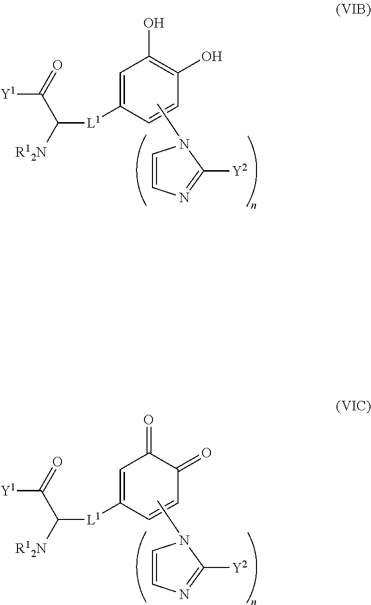

where Y¹ is a biomolecule optionally comprising one or more groups selected from, an active small molecule, an affinity tag, a fluorophore, and a metal-chelating agent; R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; each R¹ is independently selected from hydrogen, acyl, substituted acyl, alkyl, and substituted alkyl; Y² is a second biomolecule; L¹ is a linker selected from a straight or branched alkyl, a straight or branched substituted alkyl, a polyethylene glycol (PEG), a substituted PEG, and one or more peptides; and n is an integer from 1 to 3.

In certain embodiments of formula (VIB) or (VIC), n is less than 3, such as 2 or 1. In certain cases, n is 2. In certain cases, n is 1. In certain cases, the subject modified target molecule is a compound of formula (VIB). In certain cases, the subject modified target molecule is a compound of formula (VIC).

In some embodiments, the modified target molecule of formula (VIB), n is 1 and the compounds is described by any of formulae (VIB1)-(VIB3):

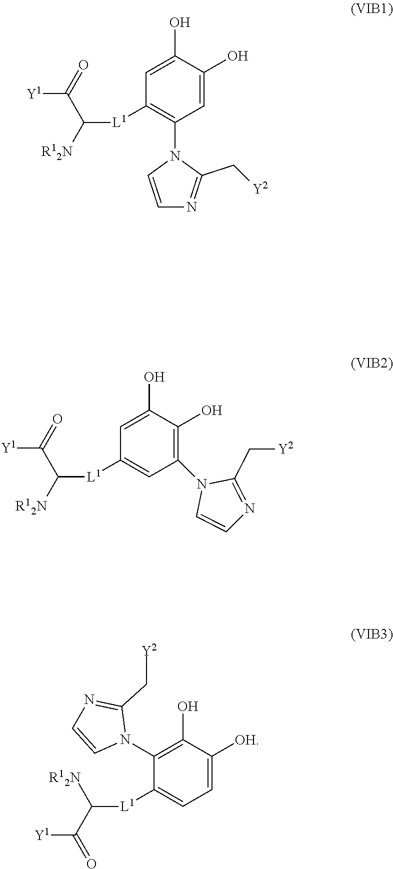

In some embodiments, the modified target molecule of formula (VIB), n is 2 and the compounds is described by any of formulae (VIB4)-(VIB5):

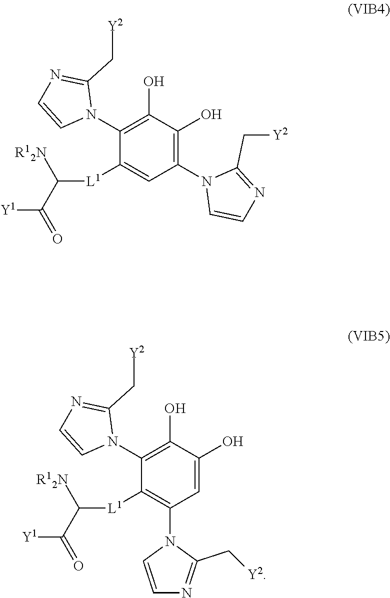

In some embodiments, the modified target molecule is of formula (VIC), n is 1, and the compound is described by any of formulae (VIC1)-(VIC3):

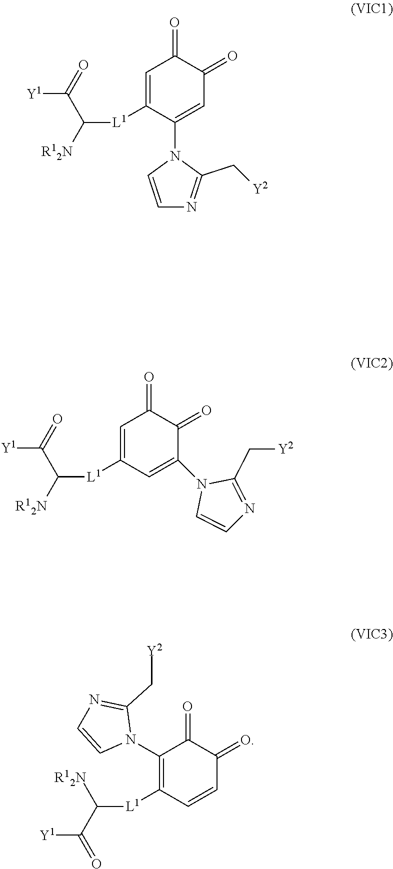

In some embodiments, the modified target molecule is of formula (VIC), n is 2, and the compound is described by any of formulae (VIC4)-(VIC5):

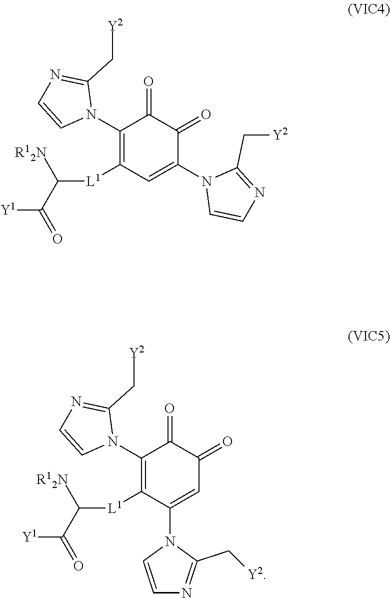

In certain embodiments of any of formulae (VB)—(VC5), at least one R¹ is hydrogen. In certain cases, both R¹ groups are hydrogen. In certain cases, one R group is hydrogen, and the other R¹ group is selected from alkyl, substituted alkyl, acyl and substituted acyl. In certain cases, one R¹ group is hydrogen and the other R¹ group is alkyl. In some cases, one R¹ group is hydrogen and the other R¹ group is substituted alkyl. In some cases, one R¹ group is hydrogen and the other R¹ group is acyl. In some cases, one R¹ group is hydrogen, and the other R¹ group is substituted acyl. In some cases the acyl group is of the formula —C(O)R⁴, wherein R⁴ is a lower alkyl group, e.g., methyl, ethyl, propyl, butyl, pentyl, or hexyl. In some cases, the substituted acyl group is of the formula —C(O)R⁴NH₂, wherein R⁴ is a lower alkyl group, e.g., methyl, ethyl, propyl, butyl, pentyl or hexyl. In some cases, the substituted acyl group is of the formula —C(O)CH₂NH₂.

In certain embodiments of any of formulae (VIB)—(VIC5), L¹ is a straight or branched alkyl. In certain cases, L¹ is a lower alkyl group, e.g., methyl, ethyl, propyl, butyl, pentyl, or hexyl. In certain cases, L¹ is a substituted alkyl group. In certain cases, L¹ is a substituted lower alkyl group. In certain cases, L¹ is a PEG or substituted PEG (e.g., as described herein). In certain other cases, L¹ is a peptide. In certain other cases, L¹ is a polypeptide. In certain cases, L¹ is a linear linker of 1-12 atoms in length, such as 1-10, 1-8 or 1-6 atoms in length, e.g., 1, 2, 3, 4, 5 or 6 atoms in length. The linker L¹ can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl). In certain cases, the linker L¹ can include a keto (C═O) group. In certain cases, the keto group together with an amino, thiol or ether group in the linker chain can provide an amido, an ester or thioester group linkage.

In certain embodiments, the linking group L¹ is a cleavable linker, e.g., as described herein.

In certain embodiments, the modified target molecule is described by any of the formulae (VID)-(VIG):

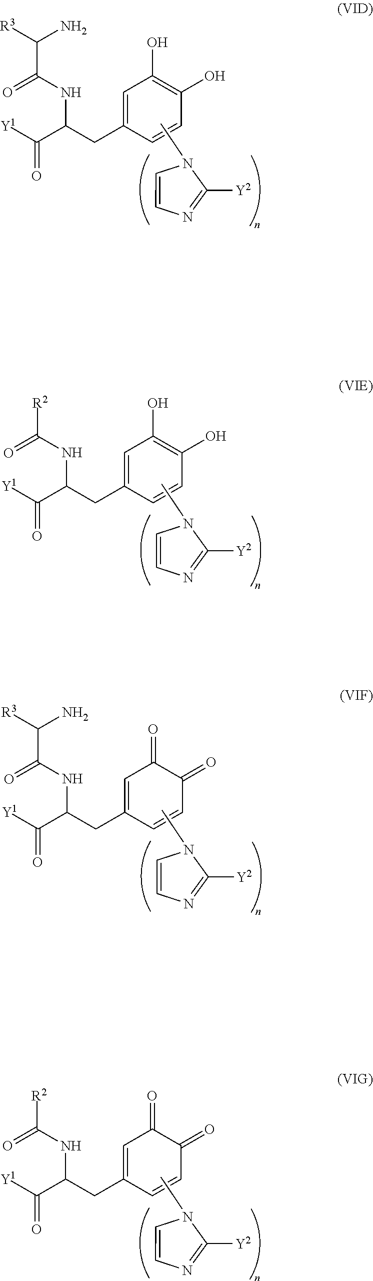

where R² is selected from alkyl, and substituted alkyl; R³ is selected from, hydrogen, alkyl substituted alkyl, a peptide, and a polypeptide; and n is an integer from 1 to 3.

In certain embodiments of any of formulas (VTD)-(VIG), n is less than 3, such as 2 or 1. In certain cases, n is 2. In certain cases, n is 1. In certain cases, the subject modified target molecule is a compound of formula (VID). In certain cases, the subject modified target molecule is a compound of formula (VIE). In certain cases, the subject modified target molecule is a compound of formula (VIF). In certain cases, the subject modified target molecule is a compound of formula (VIG).

In certain embodiments, any of formulae (VID)-(VIG) may have relative stereochemistry as shown in the following structures:

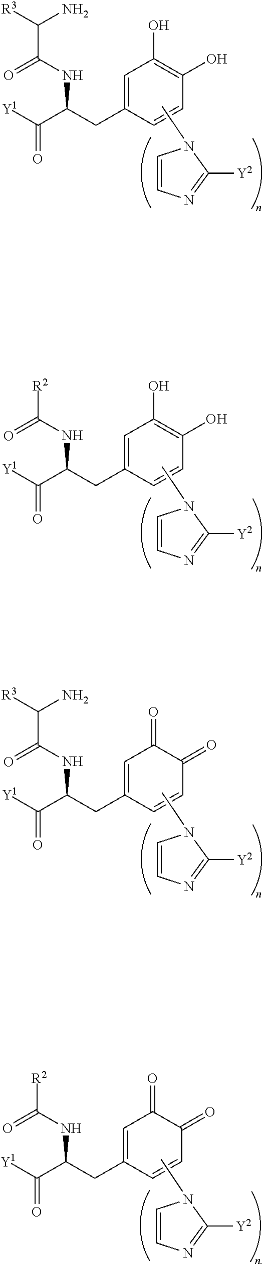

In some embodiments, the modified target molecule of formula (VD), n is 1 and the compounds is described by any of formulae (VID1)-(VID3):

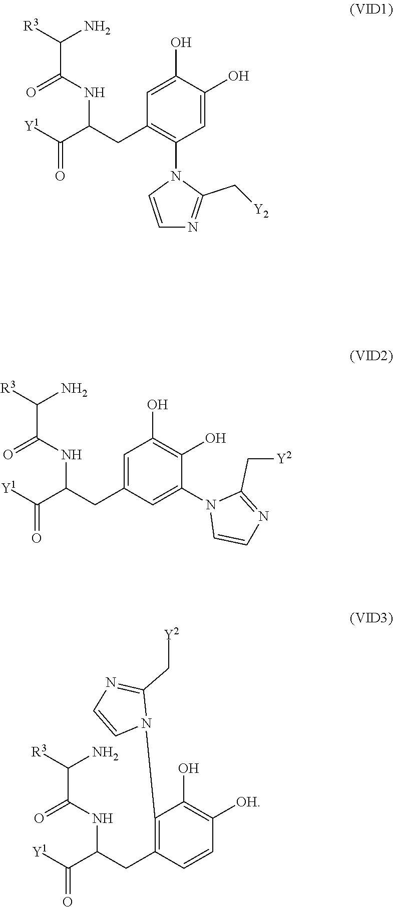

In some embodiments, the modified target molecule of formula (IVD), n is 2 and the compounds is described by any of formulae (VID4)-(VID5):

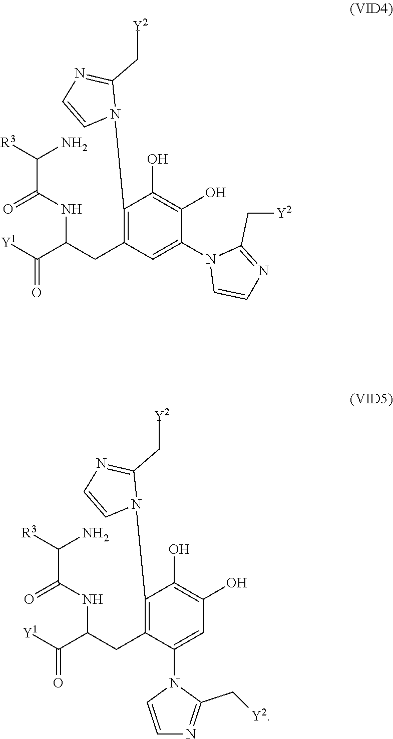

In some embodiments, the modified target molecule of formula (VIE), n is 1 and the compounds is described by any of formulae (VIE1)-(VIE3):

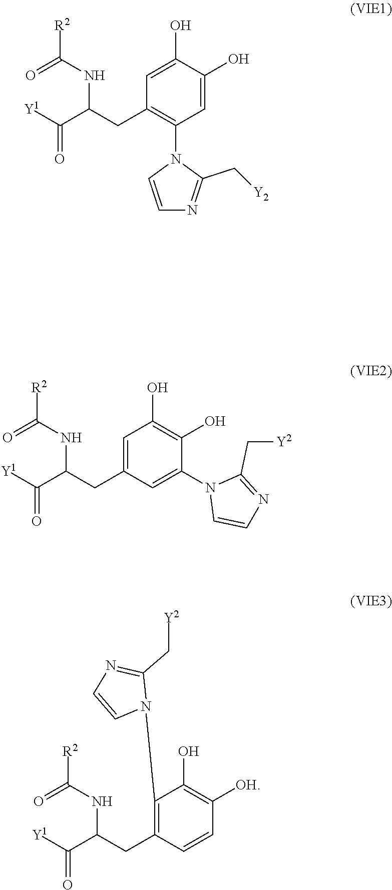

In some embodiments, the modified target molecule of formula (VIE), n is 2 and the compounds is described by any of formulae (VIE4)-(VIE5):

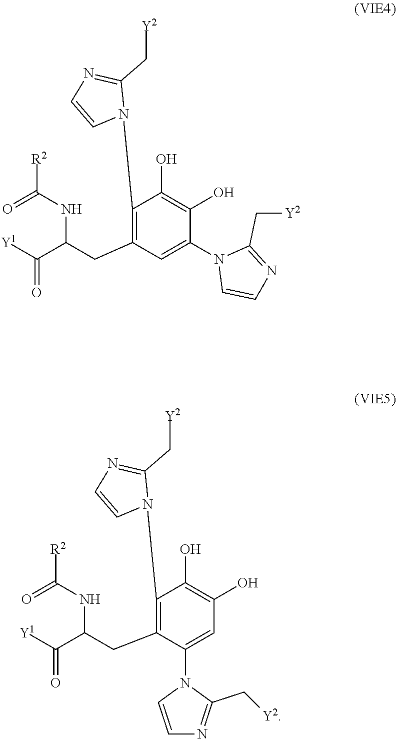

In some embodiments, the modified target molecule of formula (VIF), n is 1 and the compounds is described by any of formulae (VF1)-(VIF3):

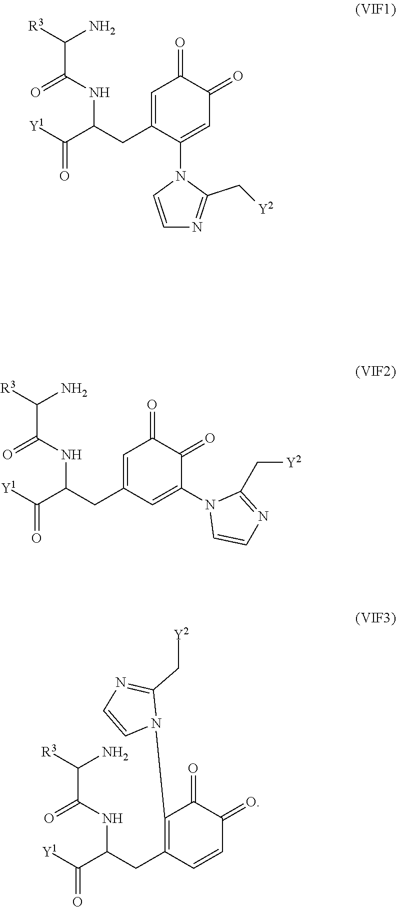

In some embodiments, the modified target molecule of formula (VIF), n is 2 and the compounds is described by any of formulae (VIF4)-(VIF5):

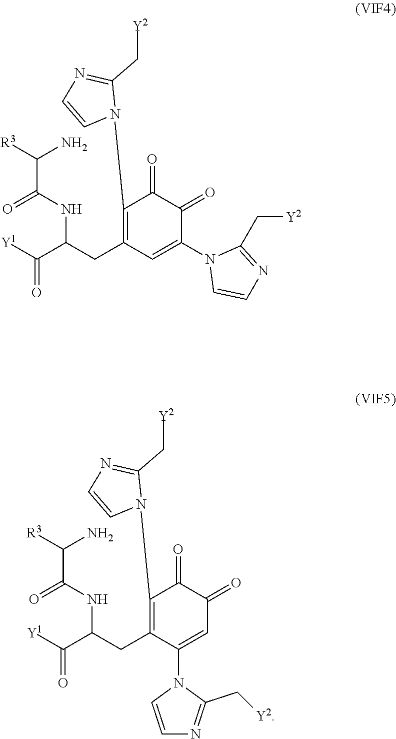

In some embodiments, the modified target molecule of formula (VIG), n is 1 and the compounds is described by any of formulae (VIG1)-(VIG3):

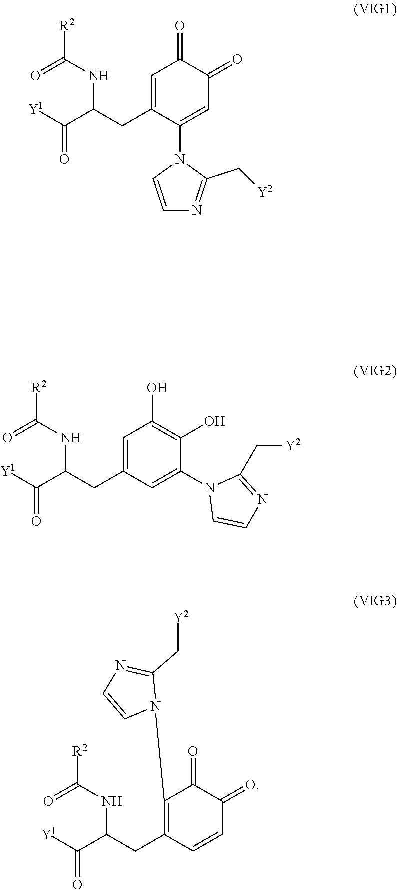

In some embodiments, the modified target molecule of formula (VIG), n is 2 and the compounds is described by any of formulae (VIG4)-(VIG5):

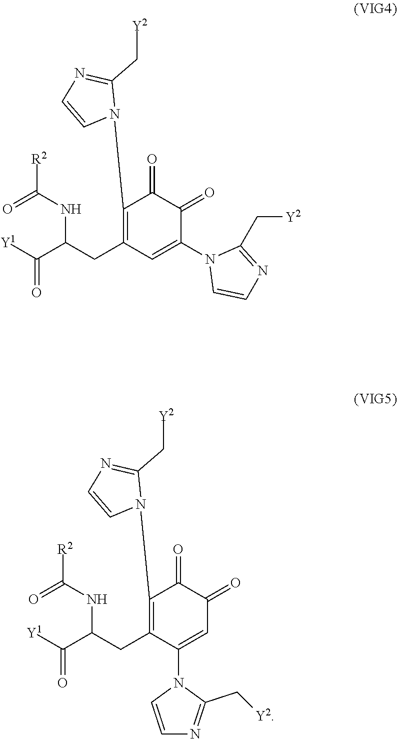

In certain embodiments of the target molecules described herein, R² is an alkyl group. In certain cases, R² is a substituted alkyl group. In certain cases, the alkyl group is a lower alkyl group, e.g., methyl, ethyl, propyl, butyl, pentyl or hexyl.

In certain embodiments of the target molecules described herein, R³ is hydrogen. In certain cases, R³ an alkyl group. In certain cases, R³ is a substituted alkyl group. In certain cases the alkyl group is a lower alkyl group, e.g., methyl, ethyl, propyl, butyl, pentyl or hexyl. In certain cases, R³ is a peptide. In certain cases, R³ is a polypeptide.

In certain embodiments, the modified target molecule is described by the formula (VIH)

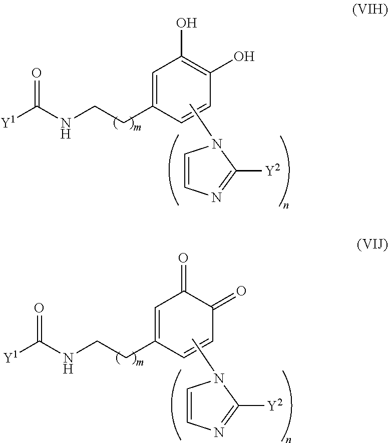

where Y¹ is a biomolecule optionally comprising one or more groups selected from, an active small molecule, an affinity tag, a fluorophore, and a metal-chelating agent; Y² is a second biomolecule; n is an integer from 1 to 3; and m is an integer from 0 to 20. In certain cases, m is 10 or less, such as 9, 8, 7, 6, 5, 4, 3, 2, 1 or 0. In certain cases, m is 5. In certain cases, m is 4. In certain cases, m is 3. In certain cases, m is 2. In certain cases, m is 1. In certain cases, m is 0.

In certain embodiments of the subject methods, the modified target molecule is described by the formula (VIK) or VIL):

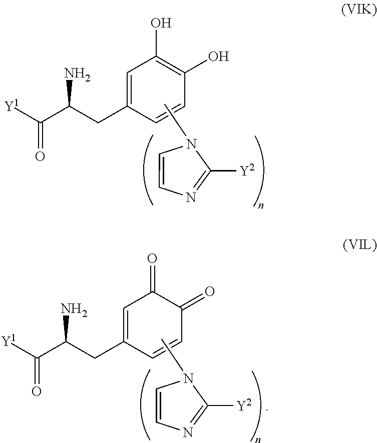

In certain embodiments of any of formulae (VIH)—(VIJ), n is less than 3, such as 2 or 1. In certain cases, n is 2. In certain cases, n is 1.

In some embodiments, the modified target molecule of formula (VIH), n is 1 and the compounds is described by any of formulae (VIH1)-(VIH3):

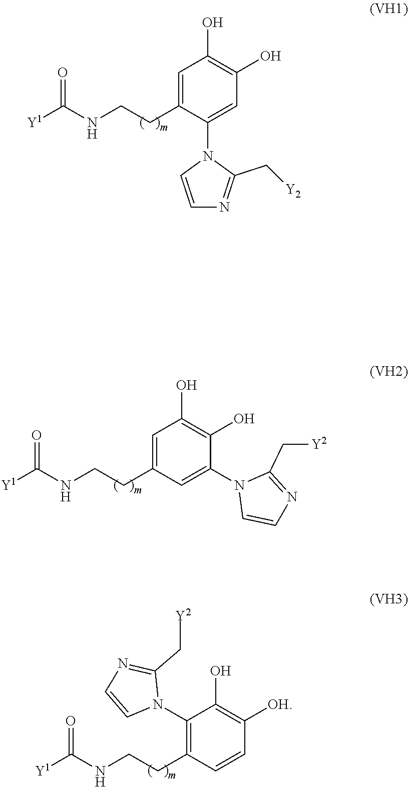

In some embodiments, the modified target molecule of formula (IVH), n is 2 and the compounds is described by any of formulae (VH4)-(VH5):

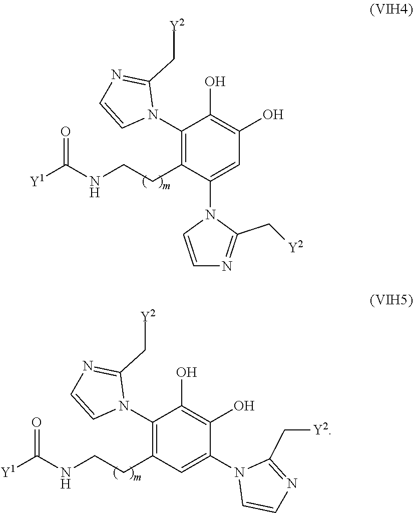

In some embodiments, the modified target molecule of formula (VJ), n is 1 and the compounds is described by any of formulae (VJ1)-(VJ3):

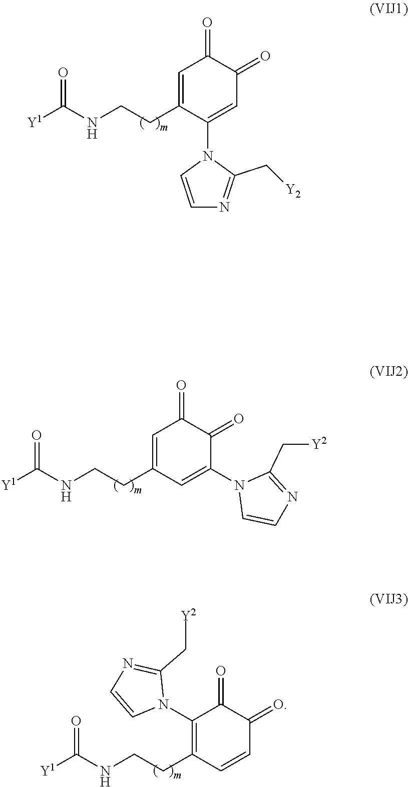

In some embodiments, the modified target molecule of formula (VIJ), n is 2 and the compounds is described by any of formulae (VIJ4)-(VIJ5):

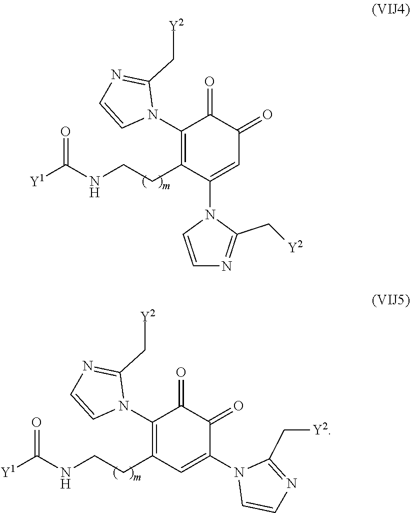

In certain embodiments, the target molecule comprising a thiol group is a CRISPR-Cas effector polypeptide (e.g., as described herein).

In certain embodiments of any one of formulae (VI) to (VIJ5), Y¹ is a polypeptide. In certain cases, the Y¹ polypeptide is selected from a fluorescent protein, an antibody, and an enzyme. In certain cases, the fluorescent protein is a green fluorescent protein. Other suitable polypeptides are described elsewhere herein.

In some embodiments of any one of the formulae of the modified target molecule described herein (e.g., formulae (IV)—(VIJ5)), an imidazole of formula (VII), or an amine of formula (VIII), Y¹ and Y² are each a polypeptide. In some embodiments of any one of the formulae of the modified target molecule described herein (e.g., formulae (IV)—(VIJ5)), an imidazole of formula (VII), or an amine of formula (VIII), Y¹ is selected from a fluorescent protein, an antibody, and an enzyme; and/or Y² is selected from a fluorescent protein, an antibody, and an enzyme. In some embodiments of any one of the formulae of the modified target molecule described herein (e.g., formulae (IV)—(VIJ5)), an imidazole of formula (VII), or an amine of formula (VIII), Y¹ or Y² is a CRISPR-Cas effector polypeptide. In some embodiments of any one of the formulae of the modified target molecule described herein (e.g., formulae (IV)—(VIJ5)), an imidazole of formula (VII), or an amine of formula (VIII), Y¹ or Y² is an antibody. In some embodiments of any one of the formulae of the modified target molecule described herein (e.g., formulae (IV)—(VIJ5)), an imidazole of formula (VII), or an amine of formula (VIII), Y¹ is an antibody and Y² is an antibody.

In some embodiments of any one of the formulae of the modified target molecule described herein (e.g., formulae (IV)—(VIJ5)), an imidazole of formula (VII), or an amine of formula (VIII), the antibody at Y¹ or Y² binds specifically to a cancer-associated antigen. In some embodiments of any one of the formulae of the modified target molecule described herein (e.g., formulae (IV)—(VIJ5)), an imidazole of formula (VII), or an amine of formula (VIII), the cancer-associated antigen is selected from the group consisting of Her-2, CD19, mesothelin, CD20, WT-1, MUC-1, BCMA, claudin-18.2, PD-L1, FLT-3, EGFR, and VEGF.

In some embodiments of any one of the formulae of the modified target molecule described herein (e.g., formulae (IV)—(VIJ5)), an imidazole of formula (VII), or an amine of formula (VIII), the antibody at Y¹ or Y² is selected from the group consisting of a single-domain antibody, an IgG1 isotype antibody or fragment thereof, an IgG2 isotype antibody or fragment thereof, an IgG3 isotype antibody or fragment thereof, an IgG4 isotype antibody or fragment thereof, an IgE isotype antibody or fragment thereof, an IgM isotype antibody or fragment thereof, and an Fc domain. In some embodiments of any one of the formulae of the modified target molecule described herein (e.g., formulae (IV)—(VIJ5)), an imidazole of formula (VII), or an amine of formula (VIII), the antibody at Y¹ or Y² is a single-domain antibody. In some embodiments of any one of the formulae of the modified target molecule described herein (e.g., formulae (IV)—(VIJ5)), an imidazole of formula (VII), or an amine of formula (VIII), the antibody at Y¹ or Y² is a single-chain Fv. In some embodiments of any one of the formulae of the modified target molecule described herein (e.g., formulae (IV)—(VIJ5)), an imidazole of formula (VII), or an amine of formula (VIII), the antibody at Y¹ or Y² is modified to include a tyrosine residue within 5 amino acids of the C-terminus amino acid of the antibody. In some embodiments of any one of the formulae of the modified target molecule described herein (e.g., formulae (IV)—(VIJ5)), an imidazole of formula (VII), or an amine of formula (VIII), the antibody at Y¹ or Y² is modified to include a C-terminal tyrosine residue.

In some embodiments of any one of the formulae of the modified target molecule described herein (e.g., formulae (IV)—(VIJ5)), an imidazole of formula (VII), or an amine of formula (VIII), Y¹ or Y² is a cell. In some embodiments of any one of the formulae of the modified target molecule described herein (e.g., formulae (IV)—(VIJ5)), an imidazole of formula (VII), or an amine of formula (VIII), Y¹ is a cell and Y² is a cell. In some embodiments of any one of the formulae of the modified target molecule described herein (e.g., formulae (IV)—(VIJ5)), an imidazole of formula (VII), or an amine of formula (VIII), the cell at Y¹ or Y² is an immune cell. In some embodiments of any one of the formulae of the modified target molecule described herein (e.g., formulae (IV)—(VIJ5)), an imidazole of formula (VII), or an amine of formula (VIII), the cell at Y¹ or Y² is an innate immune cell or an adaptive immune cell.

In some embodiments of any one of the formulae of the modified target molecule described herein (e.g., formulae (IV)—(VIJ5)), an imidazole of formula (VII), or an amine of formula (VIII), the cell at Y¹ or Y² is a natural killer (NK) cell, a macrophage, a dendritic cell, a mast cell, an eosinophil, a basophil, a neutrophil, or a monocyte. In some embodiments of any one of the formulae of the modified target molecule described herein (e.g., formulae (IV)—(VIJ5)), an imidazole of formula (VII), or an amine of formula (VIII), the cell at Y¹ or Y² is a natural killer (NK) cell.

In some embodiments of any one of the formulae of the modified target molecule described herein (e.g., formulae (IV)—(VIJ5)), an imidazole of formula (VII), or an amine of formula (VIII), the cell at Y¹ or Y² is a T-cell or a B-cell. In some embodiments of any one of the formulae of the modified target molecule described herein (e.g., formulae (IV)—(VIJ5)), an imidazole of formula (VII), or an amine of formula (VIII), the T-cell is a CD4+ cell, CD8+ cell, a helper T cell, a cytotoxic T cell, or a regulatory T cell.

In some embodiments of any one of the formulae of the modified target molecule described herein (e.g., formulae (IV)—(VIJ5)), an imidazole of formula (VII), or an amine of formula (VIII), the cell at Y¹ or Y² is a stem cell. In some embodiments of any one of the formulae of the modified target molecule described herein (e.g., formulae (IV)—(VIJ5)), an imidazole of formula (VII), or an amine of formula (VIII), the stem cell is selected from a hematopoietic stem cell, a pluripotent stem cell, and a differentiated stem cell.

In some embodiments of any one of the formulae of the modified target molecule described herein (e.g., formulae (IV)—(VIJ5)), an imidazole of formula (VII), or an amine of formula (VIII), n is 2. In some embodiments of any one of the formulae of the modified target molecule described herein (e.g., formulae (IV)—(VIJ5)), an imidazole of formula (VII), or an amine of formula (VIII), n is 1.

### Compositions

In another aspect, provided herein are compositions, including pharmaceutical compositions, comprising a target molecule comprising an imidazole of formula (VII),

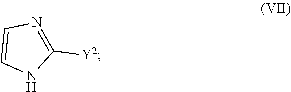

and
a biomolecule comprising a phenol moiety or a catechol moiety of formula (I):

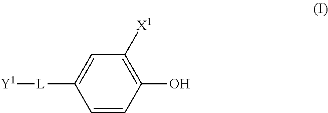

wherein:
Y¹ is a biomolecule, optionally comprising one or more moieties selected from, an active small molecule, an affinity tag, a fluorophore, and a metal-chelating agent;
X¹ is selected from hydrogen and hydroxyl;
L is an optional linker; and
Y² is a second biomolecule.

Also provided herein are compositions, including pharmaceutical compositions, comprising a target molecule comprising an amine of formula (VIII),

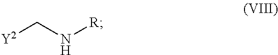

and
a biomolecule comprising a phenol moiety or a catechol moiety of formula (I):

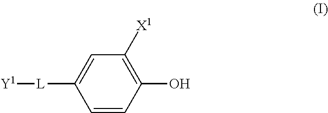

wherein:
Y¹ is a biomolecule, optionally comprising one or more moieties selected from, an active small molecule, an affinity tag, a fluorophore, and a metal-chelating agent;
X¹ is selected from hydrogen and hydroxyl;
L is an optional linker;
   Y² is a second biomolecule; and
      R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

### Cleavable Linkers

Cleavable linkers that may be employed in the subject molecules of interest include electrophilically cleavable linkers, nucleophilically cleavable linkers, photocleavable linkers, metal cleavable linkers, electrolytically-cleavable, and linkers that are cleavable under reductive and oxidative conditions. In certain cases, the cleavable linker is cleaved under acidic conditions. In certain cases, the cleavable linker is cleaved by an enzyme. In certain cases, the cleavable linker is a linker that is cleaved under reducing conditions. In certain cases, the cleavable linker is cleaved rapidly by glutathione reduction. In certain cases, the cleavable linker includes a disulfide bond. In certain cases, the cleavable linker is cleaved by a physical stimulus. In certain cases, the cleavable linker is photocleavable.

In certain cases, L or L¹ is an acid-labile linker. In certain cases, the linker cleaves at a pH of 6 or less, such as, 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, 4.9, 4.85, 4.80, 4.75, 4.7, 4.65, 4.6, 4.55, 4.5 or even less.

In certain cases, L or L¹ is a photocleavable linker. Suitable photocleavable linkers include ortho-nitrobenzyl-based linkers, phenacyl linkers, alkoxybenzoin linkers, chromium arene complex linkers, NpSSMpact linkers and pivaloylglycol linkers, as described in Guillier et al. (Chem. Rev. 2000 1000:2091-2157).

In some cases, L or L¹ is a proteolytically cleavable linker.

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (SEQ ID NO:51) (wherein, X represents an arbitrary residue; Hy, a hydrophobic residue), e.g., Pro-X-X-Hy-(Ser/Thr) (SEQ ID NO:52), e.g., Pro-Leu/Gln-Gly-Met-Thr-Ser (SEQ ID NO:53) or Pro-Leu/Gln-Gly-Met-Thr (SEQ ID NO:54). Another example of a protease cleavage site is a plasminogen activator cleavage site, e.g., a uPA or a tissue plasminogen activator (tPA) cleavage site. In some cases, the cleavage site is a furin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYTQS (SEQ ID NO:55), where the protease cleaves between the glutamine and the serine. TEV protease recognizes a linear amino acid sequence of the general formula EX₁X₂YX₃Q(G/S), where each of X₁, X₂, and X₃ is any amino acid, and where cleavage occurs between Q and G or Q and S. A TEV protease-cleavable linker can include, ENLYFQG (SEQ ID NO:56); ENLYTQS (SEQ ID NO:55); ENLYFQGGY (SEQ ID NO:57); ENLYFQS (SEQ ID NO:58); and the like. Another example of a protease cleavage site that can be included in a proteolytically cleavable linker is an enterokinase cleavage site, e.g., DDDDK (SEQ ID NO:59), where cleavage occurs after the lysine residue. Another example of a protease cleavage site that can be included in a proteolytically cleavable linker is a thrombin cleavage site, e.g., LVPR (SEQ ID NO:60). Additional suitable linkers comprising protease cleavage sites include linkers comprising one or more of the following amino acid sequences: LEVLFQGP (SEQ ID NO:61), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO:62); SLLKSRMVPNFN (SEQ ID NO:63) or SLLIARRMPNFN (SEQ ID NO:64), cleaved by cathepsin B; SKLVQASASGVN (SEQ ID NO:65) or SSYLKASDAPDN (SEQ ID NO:66), cleaved by an Epstein-Barr virus protease; RPKPQQFFGLMN (SEQ ID NO:67) cleaved by MMP-3 (stromelysin); SLRPLALWRSFN (SEQ ID NO:68) cleaved by MMP-7 (matrilysin); SPQGIAGQRNFN (SEQ ID NO:69) cleaved by MMP-9; DVDERDVRGFASFL SEQ ID NO:70) cleaved by a thermolysin-like MMP; SLPLGLWAPNFN (SEQ ID NO:71) cleaved by matrix metalloproteinase 2 (MMP-2); SLLIFRSWANFN (SEQ ID NO:72) cleaved by cathespin L; SGVVIATVIVIT (SEQ ID NO:73) cleaved by cathepsin D; SLGPQGIWGQFN (SEQ ID NO:74) cleaved by matrix metalloproteinase 1 (MMP-1); KKSPGRVVGGSV (SEQ ID NO:75) cleaved by urokinase-type plasminogen activator; PQGLLGAPGILG (SEQ ID NO:76) cleaved by membrane type 1 matrixmetalloproteinase (MT-MMP); HGPEGLRVGFYESDVMGRGHARLVHVEEPHT (SEQ ID NO:77) cleaved by stromelysin 3 (or MMP-11), thermolysin, fibroblast collagenase and stromelysin-1; GPQGLAGQRGIV (SEQ ID NO:78) cleaved by matrix metalloproteinase 13 (collagenase-3); GGSGQRGRKALE (SEQ ID NO:79) cleaved by tissue-type plasminogen activator (tPA); SLSALLSSDIFN (SEQ ID NO:80) cleaved by human prostate-specific antigen; SLPRFKIIGGFN (SEQ ID NO:81) cleaved by kallikrein (hK3); SLLGIAVPGNFN (SEQ ID NO:82) cleaved by neutrophil elastase; and FFKNIVTPRTPP (SEQ ID NO:83) cleaved by calpain (calcium activated neutral protease).

In some cases, the linker comprises a disulfide bond and is cleavable under reducing conditions, e.g., using β-mercaptoethanol, cysteine-HCl, Tris (2-carboxyethyl) phosphine hydrochloride, or another reducing agent.

In some cases, the linker comprises a dipeptide such as a valine-citrulline dipeptide or a valine-lysine dipeptide.

### Biomolecules

Biomolecules that are suitable for use in a method or conjugate of the present disclosure include polypeptides. The term “biomolecule” encompasses chemically modified polypeptides. For example, the term “biomolecule” includes a monovalent radical of i) a chemical structure of a biomolecule or ii) a chemical structure of a biomolecule substituted with a chemical functional group.

In some cases, the biomolecule is a polypeptide or other cell surface macromolecule present on the surface of a cell, e.g., a living cell (e.g., a eukaryotic cell, a mammalian cell, etc.). Thus, in some cases, the biomolecule is a cell. The term “cell” can include a cell comprising a chemically modified macromolecule, e.g., a chemically modified polypeptide. The term “cell” includes a monovalent radical of i) a chemical structure of a cell or ii) a chemical structure of a cell substituted with a chemical functional group. In some cases, the biomolecule is an antibody. The term “antibody” encompasses a chemically modified antibody. The term “antibody” includes a monovalent radical of i) a chemical structure of an antibody or ii) a chemical structure of an antibody substituted with a chemical functional group.

In some cases, the biomolecule is an antibody. Suitable antibodies are described elsewhere herein. The antibody can be any antigen-binding antibody-based polypeptide, a wide variety of which are known in the art. In some instances, the antibody is a single chain Fv (scFv). Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

An antibody can be specific for cancer-associated antigen. Cancer-associated antigens include, e.g., NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CEA (carcinoembryonic antigen), EGFR (epidermal growth factor receptor), EGFRvIIJ (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A3 (melanoma-associated antigen 3), melanoma glycoprotein, mesothelin, MUC1 (mucin 1), MUC16 (mucin-16), myelin, NKG2D (Natural Killer Group 2D) ligands, PSMA (prostate specific membrane antigen), and ROR1 (type I receptor tyrosine kinase-like orphan receptor).

Cancer-associated antigens include, e.g., 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, claudin (e.g., claudin-1, claudin-10, claudin-18 (e.g., claudin-18, isoform 2)), complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ, integrin α5β1, KC4-antigen, killer-cell immunoglobulin-like receptor (KIR), Kras, KS-1-antigen, KS1-4, LDR/FUT, Le^{gamma} macrophage migration inhibitory factor (MIF), MAGE, MAGE-3, MART-1, MART-2, mCRP, MCP-1, melanoma glycoprotein, mesothelin, MIP-IA, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

In some cases, the cancer-associated antigen is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ, IGF-R¹, IL6, KIR, PD-1, PD-L1, TRAILR1, TRAILR2, transferrin receptor, and VEGF. In some cases, the cancer-associated antigen is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the cancer-associated antigen is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma}, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

Examples of antibodies that target cancer-associated antigens and that are suitable for attachment to a target cell include, but are not limited to, abituzumab (anti-CD51), LL1 (anti-CD74), LL2 or RFB4 (anti-CD22), veltuzumab (hA20, anti-CD20), rituxumab (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fctoprotein), R¹ (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothclin).

In some cases, an antibody is modified to include a Tyr residue at or near the C-terminus of the antibody. In some cases, an antibody is modified to include a linker between the C-terminus of the antibody and a C-terminal Tyr added to the antibody. Suitable linkers include, e.g., (GGGG)n (SEQ ID NO:84), where n is an integer from 1 to 5; SGGGG (SEQ ID NO:85); (GGGGS)n (SEQ ID NO:86), where n is an integer from 1 to 5, and the like.

The following are non-limiting examples of antibodies that can be conjugated to the surface of a living cell.

### Anti-Her2

In some cases, an antibody suitable for conjugation to a target cell is an anti-Her2 antibody. In some cases, an anti-Her2 antibody comprises: a) a light chain comprising an amino acid sequence having at least 90%, at least 95%, at least 98%, at least 99%, or 100%, amino acid sequence identity to the following amino acid sequence:

DiQMTQSPSSLSASVGDRVTiTCRASQDVNTAVAWYQQKPGKAPKLLiYSASFLY SGVPSRFSGSRSGTDFTLTISSLQPEDFATYYCQQHYTTPPTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:87); and b) a heavy chain comprising an amino acid sequence having at least 90%, at least 95%, at least 98%, at least 99%, or 100%, amino acid sequence identity to the following amino acid sequence:

(SEQ ID NO: 88)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:89); and b) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:90). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:90); b) a linker; and c) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:89). Suitable linkers are described elsewhere herein and include, e.g., (GGGGS)n (SEQ ID NO: 86), where n is an integer from 1 to 10 (e.g., 1, 2, 3, 4, 5, 6, 7, 8, 9, or 10).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-Her2 antibody can comprise a VL CDR1 having the amino acid sequence RASQDVNTAVA (SEQ ID NO:91); a VL CDR2 having the amino acid sequence SASFLY (SEQ ID NO:92); a VL CDR3 having the amino acid sequence QQHYTTPP (SEQ ID NO:93); a VH CDR1 having the amino acid sequence GFNIKDTY (SEQ ID NO:94); a VH CDR2 having the amino acid sequence IYPTNGYT (SEQ ID NO:95); and a VH CDR3 having the amino acid sequence SRWGGDGFYAMDY (SEQ ID NO:96).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence:

(SEQ ID NO: 97)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSDIQMTQSPSSLS
ASVGDRVTITCRASQDVNTAVAWYQQKPGKAPKLLIYSASFLYSGVPSR
FSGSRSGTDFTLTISSLQPEDFATYYCQQHYTTPPTFGQGTKVEIK.

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence:

DIQMTQSPSSLSASVGDRVTITCKASQDVSIGVAWYQQKPGKAPKLLIYSASYRY TGVPSRFSGSGSGTDFTLTISSLQPEDFATYYCQQYYIYPYTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:98); and b) a heavy chain variable region (VH) comprising an amino acid sequence having at least 90%, at least 95%, at least 98%, at least 99%, or 100%, amino acid sequence identity to the following amino acid sequence:

(SEQ ID NO: 99)
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPG.

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCKASQDVSIGVAWYQQKPGKAPKLLIYSASYRYTGVPSRFSGS GSGTDFTLTISSLQPEDFATYYCQQYYIYPYTFGQGTKVEIK (SEQ ID NO:100); and b) a VH comprising an amino acid sequence having at least 90%, at least 95%, at least 98%, at least 99%, or 100%, amino acid sequence identity to the amino acid sequence:

(SEQ ID NO: 101)
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSS.

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the VH and V_{L} CDRs are as defined by Chothia (see, e.g., Table 1, above; and Chothia 1987).

For example, an anti-HER2 antibody can comprise a VL CDR1 having the amino acid sequence KASQDVSIGVA (SEQ ID NO:102); a VL CDR2 having the amino acid sequence SASYRY (SEQ ID NO:103); a VL CDR3 having the amino acid sequence QQYYIYPY (SEQ ID NO:104); a VH CDR1 having the amino acid sequence GFTFTDYTMD (SEQ ID NO:105); a VH CDR2 having the amino acid sequence ADVNPNSGGSIYNQRFKG (SEQ ID NO:106); and a VH CDR3 having the amino acid sequence ARNLGPSFYFDY (SEQ ID NO:107).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence:

(SEQ ID NO: 97)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSDIQMTQSPSSLS
ASVGDRVTITCRASQDVNTAVAWYQQKPGKAPKLLIYSASFLYSGVPSR
FSGSRSGTDFTLTISSLQPEDFATYYCQQHYTTPPTFGQGTKVEIK.

In some cases, an anti-Her2 antibody is a nanobody. For example, in some cases, an anti-Her2 nanobody comprises an amino acid sequence having at least 90%, at least 95%, at least 98%, at least 99%, or 100%, amino acid sequence identity to the following amino acid sequence: MEVQLVESGGGLVQAGGSLRLSCAASGITFSINTMGWYRQAPGKQRELVALISSIGDTYYADSV KGRFTISRDNAKNTVYLQMNSLKPEDTAVYYCKRFRTAAQGTDYWGQGTQVTVSSAAAHHH HHHSGGGGY (SEQ ID NO:108). As another example, in some cases, an anti-Her2 nanobody comprises an amino acid sequence having at least 90%, at least 95%, at least 98%, at least 99%, or 100%, amino acid sequence identity to the following amino acid sequence: MEVQLVESGGGLVQAGGSLRLSCAASGITFSINTMGWYRQAPGKQRELVALISSIGDTYYADSV KGRFTISRDNAKNTVYLQMNSLKPEDTAVYYCKRFRTAAQGTDYWGQGTQVTVSSAAASGGG GY (SEQ ID NO:109). As another example, in some cases, an anti-Her2 nanobody comprises an amino acid sequence having at least 90%, at least 95%, at least 98%, at least 99%, or 100%, amino acid sequence identity to the following amino acid sequence: EVQLVESGGGLVQAGGSLRLSCAASGITFSINTMGW YRQAPGKQRELV ALISSIGDTY YADSVK GRFTISRDNAKNTVYLQMNSLKPEDTAVYYCKRFRTAAQGTDYWGQGTQVTVSSAAASGGGG Y (SEQ ID NO:110). As another example, in some cases, an anti-Her2 nanobody comprises an amino acid sequence having at least 90%, at least 95%, at least 98%, at least 99%, or 100%, amino acid sequence identity to the following amino acid sequence: EVQLVESGGGLVQAGGSLRLSCAASGITFSINTMGWYRQAPGKQRELVALISSIGDTYYADSVK GRFTISRDNAKNTVYLQMNSLKPEDTAVYYCKRFRTAAQGTDYWGQGTQVTVSSSGGGGY (SEQ ID NO:111). As another example, in some cases, an anti-Her2 nanobody comprises an amino acid sequence having at least 90%, at least 95%, at least 98%, at least 99%, or 100%, amino acid sequence identity to the following amino acid sequence:

(SEQ ID NO: 112)
EVQLVESGGGLVQAGGSLRLSCAASGITFSINTMGWYRQAPGKQRELVA
LISSIGDTYYADSVKGRFTISRDNAKNTVYLQMNSLKPEDTAVYYCKRF
RTAAQGTDYWGQGTQVTVSS.

In any of the above embodiments, the antibody includes a Tyr at the C-terminus. For example, the antibody is modified to include a Tyr at the C-terminus. In any of the above embodiments, the antibody includes a linker between the C-terminal Tyr and the antibody. In any of the above embodiments, the linker is SGGGG (SEQ ID NO:85). In any of the above embodiments, the linker is GGGGS (SEQ ID NO:200).

### Anti-CD19

Anti-CD19 antibodies are known in the art; and the VH and VL, or the VH and VL CDRs, of any anti-CD19 antibody can be used in an antibody for conjugation to a target cell. See e.g., WO 2005/012493.

In some cases, an anti-CD19 antibody includes a VL CDR1 comprising the amino acid sequence KASQSVDYDGDSYLN (SEQ ID NO: 113); a VL CDR2 comprising the amino acid sequence DASNLVS (SEQ ID NO: 114); and a VL CDR3 comprising the amino acid sequence QQSTEDPWT (SEQ ID NO: 115). In some cases, an anti-CD19 antibody includes a VH CDR1 comprising the amino acid sequence SYWMN (SEQ ID NO:116); a VH CDR2 comprising the amino acid sequence QIWPGDGDTNYNGKFKG (SEQ ID NO: 117); and a VH CDR3 comprising the amino acid sequence RETTIVGRYYYAMDY (SEQ ID NO:118). In some cases, an anti-CD19 antibody includes a VL CDR1 comprising the amino acid sequence KASQSVDYDGDSYLN (SEQ ID NO:113); a VL CDR2 comprising the amino acid sequence DASNLVS (SEQ ID NO:114); a VL CDR3 comprising the amino acid sequence QQSTEDPWT (SEQ ID NO:115); a VH CDR1 comprising the amino acid sequence SYWMN (SEQ ID NO: 116); a VH CDR2 comprising the amino acid sequence QIWPGDGDTNYNGKFKG (SEQ ID NO: 117); and a VH CDR3 comprising the amino acid sequence RETTIVGRYYYAMDY (SEQ ID NO:118).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 119)
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIKGGGGSGGGGSGGGGSQVQLQQSGAELVRPGSSVKIS
CKASGYAFSSYWMNWVKQRPGQGLEWIGQIWPGDGDTNYNGKFKGKATL
TADESSSTAYMQLSSLASEDSAVYFCARRETTTVGRYYYAMDYWGQGTT
VTVS.

In any of the above embodiments, the antibody includes a Tyr at the C-terminus. For example, the antibody is modified to include a Tyr at the C-terminus. In any of the above embodiments, the antibody includes a linker between the C-terminal Tyr and the antibody. In any of the above embodiments, the linker is SGGGG (SEQ ID NO:85). In any of the above embodiments, the linker is GGGGS (SEQ ID NO:200).

### Anti-Mesothelin

Anti-mesothelin antibodies are known in the art; and the VH and VL, or the VH and VL CDRs, of any anti-mesothelin antibody can be used in an antibody for conjugation to a target cell. See, e.g., U.S. 2019/0000944; WO 2009/045957; WO 2014/031476; U.S. Pat. No. 8,460,660; US 2013/0066055; and WO 2009/068204.

In some cases, an anti-mesothelin antibody comprises: a) a light chain comprising an amino acid sequence having at least 90%, at least 95%, at least 98%, at least 99%, or 100%, amino acid sequence identity to the following amino acid sequence:

[](SEQ ID NO: 120)
DIALTQPASVSGSPGQSITISCTGTSSDIGGYNSVSWYQQHPGKAPKLM
IYGVNNRPSGVSNRFSGSKSGNTASLTISGLQAEDEADYYCSSYDIESA
TPVFGGGTKLTVLGQPKAAPSVTLFPPSSEELQANKATLVCLISDFYPG
AVTVAWKGDSSPVKAGVETTTPSKQSNNKYAASSYLSLTPEQWKSHRSY
SCQVTHEGSTVEKTVAPTESS;

and

b) a heavy chain comprising an amino acid sequence having at least 90%, at least 95%, at least 98%, at least 99%, or 100%, amino acid sequence identity to the following amino acid sequence:

(SEQ ID NO: 121)
QVELVQSGAEVKKPGESLKISCKGSGYSFTSYWIGWVRQAPGKGLEWMG
IIDPGDSRTRYSPSFQGQVTISADKSISTAYLQWSSLKASDTAMYYCAR
GQLYGGTYMDGWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTK (SEQ ID NO: 122); and b) a VH comprising an amino acid sequence having at least 90%, at least 95%, at least 98%, at least 99%, or 100%, amino acid sequence identity to the amino acid sequence:

(SEQ ID NO: 123)
QVELVQSGAEVKKPGESLKISCKGSGYSFTSYWIGWVRQAPGKGLEWMG
IIDPGDSRTRYSPSFQGQVTISADKSISTAYLQWSSLKASDTAMYYCAR
GQLYGGTYMDGWGQGTLVTVSS.

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-mesothelin antibody can comprise a VL CDR1 having the amino acid sequence TGTSSDIGGYNSVS (SEQ ID NO:124); a VL CDR2 having the amino acid sequence LMIYGVNNRPS (SEQ ID NO:125); a VL CDR3 having the amino acid sequence SSYDIESATP (SEQ ID NO:126); a VH CDR1 having the amino acid sequence GYSFTSYWIG (SEQ ID NO:127); a VH CDR2 having the amino acid sequence WMGIIDPGDSRTRYSP (SEQ ID NO:128); and a VH CDR3 having the amino acid sequence GQLYGGTYMDG (SEQ ID NO:129).

An anti-mesothelin antibody can be a scFv. As one non-limiting example, an anti-mesothelin scFv can comprise the following amino acid sequence:

(SEQ ID NO: 130)
QVQLQQSGAEVKKPGASVKVSCKASGYTFTGYYMHWVRQAPGQGLEWMG
RINPNSGGTNYAQKFQGRVTMTRDTSISTAYMELSRLRSEDTAVYYCAR
GRYYGMDVWGQGTMVTVSSGGGGSGGGGSGGGGSGGGGSEIVLTQSPAT
LSLSPGERATISCRASQSVSSNFAWYQQRPGQAPRLLIYDASNRATGIP
PRFSGSGSGTDFTLTISSLEPEDFAAYYCHQRSNWLYTFGQGTKVDIK,
where VH CDR1, CDR2, and CDR3 are underlined;
and VL CDR1, CDR2, and CDR3 are bolded and
underlined.

As one non-limiting example, an anti-mesothelin scFv can comprise the following amino acid sequence:

(SEQ ID NO: 131)
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
DLRRTVVTPRAYYGMDVWGQGTTVTVSSGGGGSGGGGSGGGGSGGGGSD
IQLTQSPSTLSASVGDRVTITCQASQDISNSLNWYQQKAGKAPKLLIYD
ASTLETGVPSRFSGSGSGTDFSFTISSLQPEDIATYYCQQHDNLPLTFG
QGTKVEIK,
where VH CDR1, CDR2, and CDR3 are underlined;
and VL CDR1, CDR2, and CDR3 are bolded and
underlined.

In any of the above embodiments, the antibody includes a Tyr at the C-terminus. For example, the antibody is modified to include a Tyr at the C-terminus. In any of the above embodiments, the antibody includes a linker between the C-terminal Tyr and the antibody. In any of the above embodiments, the linker is SGGGG (SEQ ID NO:85). In any of the above embodiments, the linker is GGGGS (SEQ ID NO:200).

### Anti-BCMA

Anti-BCMA (B-cell maturation antigen) antibodies are known in the art; and the VH and VL, or the VH and VL CDRs, of any anti-BCMA antibody can be used in an antibody for conjugation to a target cell. See, e.g., WO 2014/089335; US 2019/0153061; and WO 2017/093942.

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 132)
[]QSVLTQPPSASGTPGQRVTISCSGSSSNIGSNTVNWYQQLPGTAPKLLI
FNYHQRPSGVPDRESGSKSGSSASLAISGLQSEDEADYYCAAWDDSLNG
WVFGGGTKLTVLGQPKAAPSVTLFPPSSEELQANKATLVCLISDFYPGA
VTVAWKADSSPVKAGVETTTPDSKQSNNKYAASSYLSLTPEQWKSHRSY
SCQVTHEGSTVEKTVAPTECS;

b) a heavy chain comprising an amino acid sequence having at least 90%, at least 95%, at least 98%, at least 99%, or 100%, amino acid sequence identity to the following amino acid sequence:

(SEQ ID NO: 133)
EVQLVESGGGLVKPGGSLRLSCAASGFTFGDYALSWFRQAPGKGLEWVG
VSRSKAYGGTTDYAASVKGRFTISRDDSKSTAYLQMNSLKTEDTAVYYC
ASSGYSSGWTPFDYWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK.

In some cases, an anti-BCMA antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence:

(SEQ ID NO: 134)
QSVLTQPPSASGTPGQRVTISCSGSSSNIGSNTVNWYQQLPGTAPKLLI
FNYHQRPSGVPDRFSGSKSGSSASLAISGLQSEDEADYYCAAWDDSLNG
WVFGGGTKLTVLG;

and b) a VH comprising an amino acid sequence having at least 90%, at least 95%, at least 98%, at least 99%, or 100%, amino acid sequence identity to the amino acid sequence:

(SEQ ID NO: 135)
EVQLVESGGGLVKPGGSLRLSCAASGFTFGDYALSWFRQAPGKGLEWVG
VSRSKAYGGTTDYAASVKGRFTISRDDSKSTAYLQMNSLKTEDTAVYYC
ASSGYSSGWTPFDYWGQGTLVTVSSASTKGPSV.

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-BCMA antibody can comprise a VL CDR1 having the amino acid sequence SSNIGSNT (SEQ ID NO:136), a VL CDR2 having the amino acid sequence NYH, a VL CDR3 having the amino acid sequence AAWDDSLNGWV (SEQ ID NO:137)), a VH CDR1 having the amino acid sequence GFTFGDYA (SEQ ID NO:138), a VH CDR2 having the amino acid sequence SRSKAYGGTT (SEQ ID NO:139), and a VH CDR3 having the amino acid sequence ASSGYSSGWTPFDY (SEQ ID NO:140).

An anti-BCMA antibody can be a scFv. As one non-limiting example, an anti-BCMA scFv can comprise the following amino acid sequence:

(SEQ ID NO: 141)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYNGYDVLDNWGQGTLVTVSSGGGGSGGGGSGGGGSGGGGSDIQMTQ
SPSSLSASVGDRVTITCSASQDISNYLNWYQQKPGKAPKLLIYYTSNLH
SGVPSRFSGSGSGTDFTLTISSLQPEDFATYYCQQYRKLPWTFGQGTKL
EIKR.

As another example, an anti-BCMA scFv can comprise the following amino acid sequence:

(SEQ ID NO: 142)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKRGGGGSGGGGSGGGGSGGGGSQVQLVQSGAEVKKPGSSVK
VSCKASGGTFSNYWMHWVRQAPGQGLEWMGATYRGHSDTYYNQKFKGRV
TITADKSTSTAYMELSSLRSEDTAVYYCARGAIYNGYDVLDNWGQGTLV
TVSS.

In some cases, an anti-BCMA antibody can comprise a VL CDR1 having the amino acid sequence SASQDISNYLN (SEQ ID NO:143); a VL CDR2 having the amino acid sequence YTSNLHS (SEQ ID NO:144); a VL CDR3 having the amino acid sequence QQYRKLPWT (SEQ ID NO:145); a VH CDR1 having the amino acid sequence NYWMH (SEQ ID NO:146); a VH CDR2 having the amino acid sequence ATYRGHSDTYYNQKFKG (SEQ ID NO:147); and a VH CDR3 having the amino acid sequence GAIYNGYDVLDN (SEQ ID NO:148).

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 149)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKR.

In some cases, an anti-BCMA antibody comprises: a) a heavy chain comprising an amino acid sequence having at least 90%, at least 95%, at least 98%, at least 99%, or 100%, amino acid sequence identity to the following amino acid sequence:

(SEQ ID NO: 150)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS.

In some cases, an anti-BCMA antibody (e.g., an antibody referred to in the literature as belantamab) comprises a light chain comprising the amino acid sequence: DIQMTQSPSSLSASVGDRVTITCSASQDISNYLNWYQQKPGKAPKLLIYYTSNLHSGVPSRFSGS GSGTDFTLTISSLQPEDFATYYCQQYRKLPWTFGQGTKLEIKR (SEQ ID NO:149); and a heavy chain comprising the amino acid sequence:

(SEQ ID NO: 150)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS.

In any of the above embodiments, the antibody includes a Tyr at the C-terminus. For example, the antibody is modified to include a Tyr at the C-terminus. In any of the above embodiments, the antibody includes a linker between the C-terminal Tyr and the antibody. In any of the above embodiments, the linker is SGGGG (SEQ ID NO:85). In any of the above embodiments, the linker is GGGGS (SEQ ID NO:200).

### Anti-MUC1

In some cases, a suitable antibody for conjugating to a target cell is an antibody specific for MUC1. For example, a suitable antibody for conjugating to a target cell can be specific for a MUC1 polypeptide present on a cancer cell. In some cases, the antibody is specific for the cleaved form of MUC1; see, e.g., Fessler et al. (2009) Breast Cancer Res. Treat. 118:113. In some cases, the antibody is specific for a glycosylated MUC1 peptide; see, e.g., Naito et al. (2017) ACS Omega 2:7493; and U.S. Pat. No. 10,017,580.

As one non-limiting example, a suitable antibody for conjugating to a target cell can be a single-chain Fv specific for MUC1. See, e.g., Singh et al. (2007) Mol. Cancer Ther. 6:562; Thie et al. (2011) PLoSOne 6:e15921; Imai et al. (2004) Leukemia 18:676; Posey et al. (2016) Immunity 44:1444; EP3130607; EP3164418; WO 2002/044217; and US 2018/0112007. In some cases, a suitable antibody for conjugating to a target cell is a scFv specific for the MUC1 peptide VTSAPDTRPAPGSTAPPAHG (SEQ ID NO:199). In some cases, a suitable antibody for conjugating to a target cell is a scFv specific for the MUC1 peptide SNIKFRPGSVVVQLTLAFREGTINVHDVETQFNQYKTEAASRY (SEQ ID NO:151). In some cases, an antibody suitable for conjugation to a target cell is a scFv specific for the MUC1 peptide SVVVQLTLAFREGTINVHDVETQFNQYKTEAASRY (SEQ ID NO:152). In some cases, a suitable antibody for conjugating to a target cell is a scFv specific for the MUC1 peptide LAFREGTINVHDVETQFNQY (SEQ TD NO:153). In some cases, a suitable antibody for conjugating to a target cell is a scFv specific for the MUC1 peptide SNIKFRPGSVVVQLTLAAFREGTIN (SEQ ID NO:154).

As an example, an anti-MUC1 antibody can comprise: a VH CDR1 having the amino acid sequence RYGMS (SEQ ID NO:155); a VH CDR2 having the amino acid sequence TISGGGTYIYYPDSVKG (SEQ ID NO:156); a VH CDR3 having the amino acid sequence DNYGRNYDYGMDY (SEQ ID NO:157); a VL CDR1 having the amino acid sequence SATSSVSYIH (SEQ ID NO:158); a VL CDR2 having the amino acid sequence STSNLAS (SEQ ID NO:159); and a VL CDR3 having the amino acid sequence QQRSSSPFT (SEQ ID NO:160). See, e.g., US 2018/0112007.

As another example, an anti-MUC1 antibody can comprise a VH CDR1 having the amino acid sequence GYAMS (SEQ ID NO:161); a VH CDR2 having the amino acid sequence TISSGGTYIYYPDSVKG (SEQ ID NO:162); a VH CDR3 having the amino acid sequence LGGDNYYEYFDV (SEQ ID NO:163); a VL CDR1 having the amino acid sequence RASKSVSTSGYSYMH (SEQ ID NO:164); a VL CDR2 having the amino acid sequence LASNLES (SEQ ID NO:165); and a VL CDR3 having the amino acid sequence QHSRELPFT (SEQ ID NO:166). See, e.g., US 2018/0112007.

As another example, an anti-MUC1 antibody can comprise a VH CDR1 having the amino acid sequence DYAMN (SEQ ID NO:167); a VH CDR2 having the amino acid sequence VISTFSGNINFNQKFKG (SEQ ID NO:168); a VH CDR3 having the amino acid sequence SDYYGPYFDY (SEQ ID NO:169); a VL CDR1 having the amino acid sequence RSSQTIVHSNGNTYLE (SEQ ID NO:170); a VL CDR2 having the amino acid sequence KVSNRFS (SEQ ID NO:171); and a VL CDR3 having the amino acid sequence FQGSHVPFT (SEQ ID NO:172). See, e.g., US 2018/0112007.

As another example, an anti-MUC1 antibody can comprise a VH CDR1 having the amino acid sequence GYAMS (SEQ ID NO:161); a VH CDR2 having the amino acid sequence TISSGGTYIYYPDSVKG (SEQ ID NO:162); a VH CDR3 having the amino acid sequence LGGDNYYEY (SEQ ID NO:173); a VL CDR1 having the amino acid sequence TASKSVSTSGYSYMH (SEQ ID NO:174); a VL CDR2 having the amino acid sequence LVSNLES (SEQ ID NO:175); and a VL CDR3 having the amino acid sequence QHIRELTRSE (SEQ ID NO:176). See, e.g., US 2018/0112007.

In any of the above embodiments, the antibody includes a Tyr at the C-terminus. For example, the antibody is modified to include a Tyr at the C-terminus. In any of the above embodiments, the antibody includes a linker between the C-terminal Tyr and the antibody. In any of the above embodiments, the linker is SGGGG (SEQ ID NO:85). In any of the above embodiments, the linker is GGGGS (SEQ ID NO:200).

### Anti-MUC16

In some cases, a suitable antibody for conjugating to a target cell is an antibody specific for MUC16 (also known as CA125). See, e.g., Yin et al. (2002) Int. J. Cancer 98:737. For example, an antibody suitable for conjugation to a target cell TP can be specific for a MUC16 polypeptide present on a cancer cell. See, e.g., US 2018/0118848; and US 2018/0112008. In some cases, a MUC16-specific antibody is a scFv. In some cases, a MUC16-specific antibody is a nanobody.

As one example, an anti-MUC16 antibody can comprise a VH CDR1 having the amino acid sequence GFTFSNYY (SEQ ID NO:177); a VH CDR2 having the amino acid sequence ISGRGSTI (SEQ ID NO:178); a VH CDR3 having the amino acid sequence VKDRGGYSPY (SEQ ID NO:179); a VL CDR1 having the amino acid sequence QSISTY (SEQ ID NO:180); a VL CDR2 having the amino acid sequence TAS; and a VL CDR3 having the amino acid sequence QQSYSTPPIT (SEQ ID NO:181). See, e.g., US 2018/0118848.

In any of the above embodiments, the antibody includes a Tyr at the C-terminus. For example, the antibody is modified to include a Tyr at the C-terminus. In any of the above embodiments, the antibody includes a linker between the C-terminal Tyr and the antibody. In any of the above embodiments, the linker is SGGGG (SEQ ID NO:85). In any of the above embodiments, the linker is GGGGS (SEQ ID NO:200).

### Anti-Claudin-18.2

In some cases, a suitable antibody for conjugating to a target cell is an antibody specific for claudin-18 isoform 2 (“claudin-18.2”). See, e.g., WO 2013/167259. In some cases, a claudin-18.2-specific antibody suitable for conjugation to a target cell is a scFv. In some cases, a claudin-18.2-specific antibody is a nanobody. In some cases, a suitable antibody for conjugating to a target cell is an antibody that is specific for TEDEVQSYPSKHDYV (SEQ ID NO:182) or EVQSYPSKHDYV (SEQ ID NO:183).

As one example, an anti-claudin-18.2 antibody can comprise a VH CDR1 having the amino acid sequence GYTFTDYS (SEQ ID NO:184); a VH CDR2 having the amino acid sequence INTETGVP (SEQ ID NO:185); a VH CDR3 having the amino acid sequence ARRTGFDY (SEQ ID NO:186); a VL CDR1 having the amino acid sequence KNLLHSDGITY (SEQ ID NO:187); a VL CDR2 having the amino acid sequence RVS; and a VL CDR3 having the amino acid sequence VQVLELPFT (SEQ ID NO:188).

As another example, an anti-claudin-18.2 antibody can comprise a VH CDR1 having the amino acid sequence GFTFSSYA (SEQ ID NO:189); a VH CDR2 having the amino acid sequence ISDGGSYS (SEQ ID NO:190); a VH CDR3 having the amino acid sequence ARDSYYDNSYVRDY (SEQ ID NO:191); a VL CDR1 having the amino acid sequence QDINTF (SEQ ID NO:192); a VL CDR2 having the amino acid sequence RTN; and a VL CDR3 having the amino acid sequence LQYDEFPLT (SEQ ID NO:193).

In any of the above embodiments, the antibody includes a Tyr at the C-terminus. For example, the antibody is modified to include a Tyr at the C-terminus. In any of the above embodiments, the antibody includes a linker between the C-terminal Tyr and the antibody. In any of the above embodiments, the linker is SGGGG (SEQ ID NO:85). In any of the above embodiments, the linker is GGGGS (SEQ ID NO:200).

### Treatment Methods

The present disclosure provides a method of treating cancer in an individual, the method comprising: a) modifying an immune cell obtained from the individual with a biomolecule, wherein the biomolecule is an antibody specific for a cancer-associated antigen, wherein said modifying is carried out using a method of the present disclosure, and wherein said modifying produces a modified immune cell comprising the conjugated to the surface of the immune cell; and b) administering the modified immune cell to the individual.

In some cases, the immune cell obtained from the individual is an NK cell. In some cases, the immune cell obtained from the individual is a cytotoxic T cell (e.g., a CD8⁺ cytotoxic T cell).

In some cases, the method further comprises administering one or more cancer chemotherapeutic agents.

In some cases, the method further comprises administering an immune checkpoint inhibitor to the individual. Immune checkpoint inhibitors include antibodies that bind to immune checkpoint polypeptides such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA. Examples of immune checkpoint inhibitors include nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (Medlmmune/AZ), CP-870,893 (Genentech), Mogamulizumab (Kyowa Hakko Kirin), Varlilumab (CelIDex Therapeutics), Avelumab (EMD Serono), and Galiximab (Biogen Idec).

Cancers that can be treated with a method of the present disclosure include any cancer that can be targeted with an antibody conjugated to an immune cell. Cancers that can be treated with a method of the present disclosure include carcinomas, sarcomas, melanoma, leukemias, and lymphomas. Cancers that can be treated with a method of the present disclosure include solid tumors. Cancers that can be treated with a method of the present disclosure include metastatic cancers.

Carcinomas that can treated by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be treated by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be treated according to the methods disclosed herein include atypical meningioma, islet cell carcinoma, medullary carcinoma of the thyroid, mesenchymoma, hepatocellular carcinoma, hepatoblastoma, clear cell carcinoma of the kidney, and neurofibroma mediastinum.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### Aspects Set A

Aspect 1. A method for attachment of a biomolecule to a living cell, the method comprising:
contacting a cell in vitro with a biomolecule comprising a reactive moiety, wherein the cell comprises on its surface a cell surface molecule comprising a thiol, an amine, or an imidazole moiety;
wherein the biomolecule comprising the reactive moiety is generated by reaction of a biomolecule comprising a phenol moiety or a catechol moiety with an enzyme capable of oxidizing the phenol or catechol moiety; and wherein said contacting is under conditions sufficient for conjugation of the cell surface molecule to the biomolecule, thereby producing a modified cell.

Aspect 2. The method of aspect 1, wherein the biomolecule is an antibody.

Aspect 3. The method of aspect 2, wherein the antibody binds specifically to a cancer-associated antigen.

Aspect 4. The method of aspect 3, wherein the cancer-associated antigen is Her-2, CD19, mesothelin, CD20, WT-1, MUC-1, or BCMA.

Aspect 5. The method of any one of aspects 2-4, wherein the antibody is a single-domain antibody (a “nanobody”).

Aspect 6. The method of any one of aspects 2-4, wherein the antibody is a single-chain Fv.

Aspect 7. The method of any one of aspects 2-6, wherein the antibody is modified to include a C-terminal tyrosine residue.

Aspect 8. The method of any one of aspects 1-7, wherein the enzyme is a tyrosinase polypeptide.

Aspect 9. The method of aspect 8, wherein the tyrosinase polypeptide comprises an amino acid sequence having at least 75% amino acid sequence identity to the abTYR amino acid sequence depicted in FIG. 9 or FIG. 10.

Aspect 10. The method of aspect 8, wherein the tyrosinase polypeptide comprises an amino acid sequence having at least 75% amino acid sequence identity to any one of the amino acid sequences depicted in any one of FIG. 11A-11Z and 11AA-11VV.

Aspect 11. The method of any one of aspects 1 to 10, wherein the phenol moiety is present in a tyrosine residue.

Aspect 12. The method of any one of aspects 1 to 11, wherein the thiol moiety is present in a cysteine residue.

Aspect 13. The method of any one of aspects 1 to 11, wherein the amine moiety is present in a lysine residue.

Aspect 14. The method of any one of aspects 1 to 11, wherein the imidazole moiety is present in a histidine residue.

Aspect 15. The method of any one of aspects 1 to 14, wherein the biomolecule comprises one or more moieties selected from a fluorophore, an active small molecule, and an affinity tag.

Aspect 16. The method of any one of aspects 1 to 15, wherein the reactive moiety is an orthoquinone or a semi-quinone radical, or a combination thereof.

Aspect 17. The method of any one of aspects 1-16, wherein the cell is a natural killer cell.

Aspect 18. The method of any one of aspects 1-16, wherein the cell is a T-cell.

Aspect 19. A method of treating a cancer in an individual, the method comprising:
a) modifying an immune cell obtained from the individual with a biomolecule, wherein the biomolecule is an antibody specific for a cancer-associated antigen, wherein said modifying is carried out using a method of any one of aspects 1-18, and wherein said modifying produces a modified immune cell comprising the conjugated to the surface of the immune cell; and
b) administering the modified immune cell to the individual.

Aspect 20. The method of aspect 19, wherein the modified immune cell is a modified natural killer cell.

Aspect 21. The method of aspect 19, wherein the modified immune cell is a modified cytotoxic T cell.

Aspect 22. The method of any one of aspects 19-21, comprising administering to the individual a cancer chemotherapeutic agent.

Aspect 23. The method of any one of aspects 19-21, comprising administering to the individual an immune checkpoint inhibitor.

### Aspects Set B

Aspect 1. A compound of formula (VI) or (VIA):

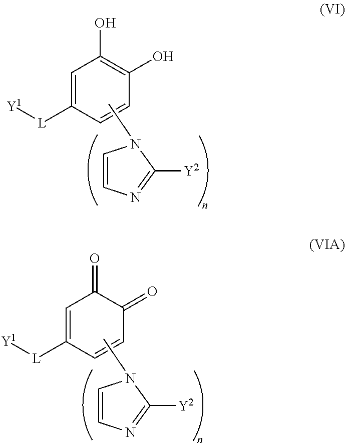

or a salt thereof, wherein:
Y¹ is a biomolecule, optionally comprising one or more moieties selected from, an active small molecule, an affinity tag, a fluorophore, and a metal-chelating agent;
L is an optional linker;
Y² is a second biomolecule; and
n is an integer from 1 to 3.

Aspect 2. The compound of aspect 1, wherein the compound is a compound of a formula selected from (VID)-(VIG):

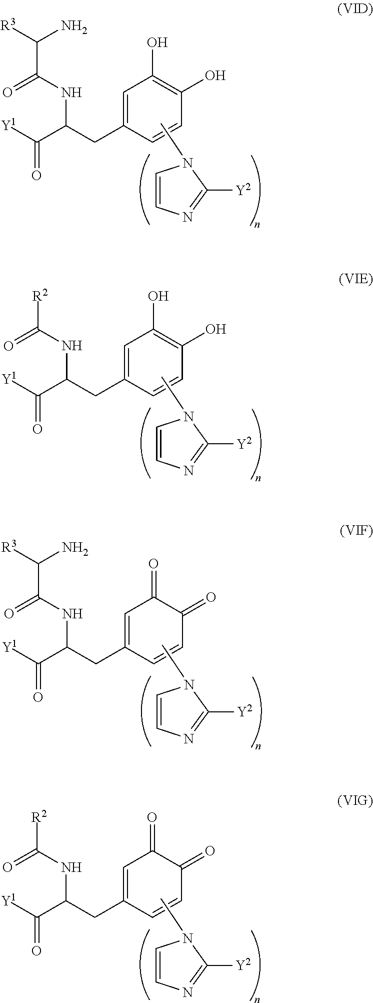

or a salt thereof, wherein:
R² is selected from alkyl and substituted alkyl;
R³ is selected from the group consisting of hydrogen, alkyl substituted alkyl, a peptide, and a polypeptide;
n is an integer from 1 to 3; and
the rest of the variables are as defined in aspect 1.

Aspect 3. The compound of aspect 1, wherein the compound is a compound of formula (VIH) or (VIJ):

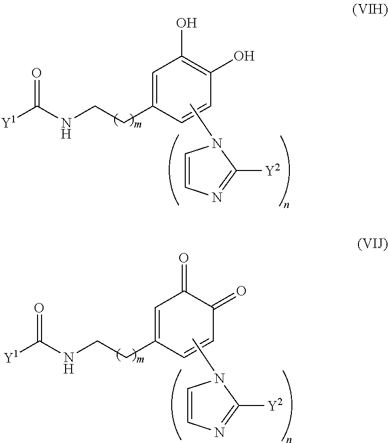

or a salt thereof, wherein:
Y¹ is a biomolecule, optionally comprising one or more groups selected from, an active small molecule, an affinity tag, a fluorophore, and a metal-chelating agent;
Y² is a second biomolecule;
n is an integer from 1 to 3; and
m is an integer from 0 to 20.

Aspect 4. A compound of formula (V) or (VA):

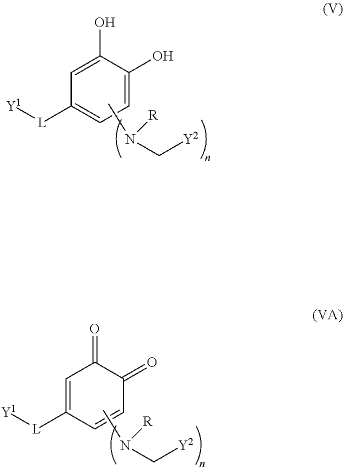

or a salt thereof, wherein:
Y¹ is a biomolecule, optionally comprising one or more moieties selected from, an active small molecule, an affinity tag, a fluorophore, and a metal-chelating agent;
L is an optional linker;
Y² is a second biomolecule;
R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
   n is an integer from 1 to 3.

Aspect 5. The compound of aspect 4, wherein the compound is a compound of a formula selected from (VD)-(VG):

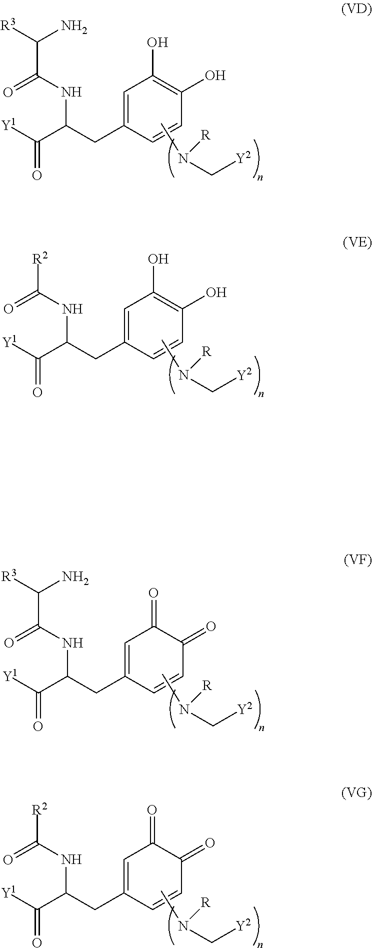

or a salt thereof, wherein:
R² is selected from alkyl, and substituted alkyl;
R³ is selected from, hydrogen, alkyl substituted alkyl, a peptide, and a polypeptide;
n is an integer from 1 to 3; and
the rest of the variables are as defined in aspect 4.

Aspect 6. The compound of aspect 2 or 5, wherein R³ is a polypeptide.

Aspect 7. The compound of aspect 4, wherein the compound is a compound of formula selected from

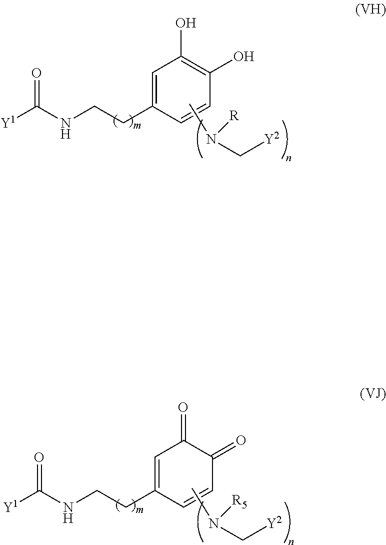

or a salt thereof, wherein:
is an integer from 1 to 3;
m is an integer from 0 to 20; and
the rest of the variables are as defined in aspect 4.

Aspect 8. The compound of any one of aspects 4-7, wherein R is hydrogen.

Aspect 9. The compound of aspect 3 or 7, wherein m is 10 or less.

Aspect 10. The compound of aspect 1 or 4, wherein Lis a cleavable linker.

Aspect 11. The compound of any one of aspects 1-10, wherein Y¹ and Y² are each a polypeptide.

Aspect 12. The compound of any one of aspects 1-11, wherein Y¹ is selected from a fluorescent protein, an antibody, and an enzyme; and/or Y² is selected from a fluorescent protein, an antibody, and an enzyme.

Aspect 13. The compound of any one of aspects 1-12, wherein Y¹ or Y² is a CRISPR-Cas effector polypeptide.

Aspect 14. The compound of any one of aspects 1-13, wherein Y¹ or Y² is an antibody.

Aspect 15. The compound of any one of aspects 1-14, wherein Y¹ is an antibody and Y² is an antibody.

Aspect 16. The compound of any one of aspects 12-15, the antibody at Y¹ or Y² binds specifically to a cancer-associated antigen.

Aspect 17. The compound of aspect 16, wherein the cancer-associated antigen is selected from the group consisting of Her-2, CD19, mesothelin, CD20, WT-1, MUC-1, BCMA, claudin-18.2, PD-L1, FLT-3, EGFR, and VEGF.

Aspect 18. The compound of any one of aspects 12-17, wherein the antibody at Y¹ or Y² is selected from the group consisting of a single-domain antibody, an IgG1 isotype antibody or fragment thereof, an IgG2 isotype antibody or fragment thereof, an IgG3 isotype antibody or fragment thereof, an IgG4 isotype antibody or fragment thereof, an IgE isotype antibody or fragment thereof, an IgM isotype antibody or fragment thereof, and an Fc domain.

Aspect 20. The compound of any one of aspects 12-18, wherein the antibody at Y¹ or Y² is a single-domain antibody.

Aspect 20. The compound of any one of aspects 12-19, wherein the antibody at Y¹ or Y² is a single-chain Fv or a nanobody.

Aspect 21. The compound of any one of aspects 12-20, wherein the antibody at Y¹ or Y² is modified to include a tyrosine residue within 5 amino acids of the C-terminus amino acid of the antibody.

Aspect 22. The compound of any one of aspects 12-20, wherein the antibody at Y¹ or Y² is modified to include a C-terminal tyrosine residue.

Aspect 23. The compound of any one of aspects 1-14 and 16-22, wherein Y¹ or Y² is a cell.

Aspect 24. The compound of any one of aspects 1-13 and 23, wherein Y¹ is a cell and Y² is a cell.

Aspect 25. The compound of aspect 23 or 24, wherein the cell at Y¹ or Y² is an immune cell.

Aspect 26. The compound of any one of aspects 23-25, wherein the cell at Y¹ or Y² is an innate immune cell or an adaptive immune cell.

Aspect 27. The compound of any one of aspects 23-26, wherein the cell at Y¹ or Y² is a natural killer (NK) cell, a macrophage, a dendritic cell, a mast cell, an eosinophil, a basophil, a neutrophil, or a monocyte.

Aspect 28. The compound of any one of aspects 23-27, wherein the cell at Y¹ or Y² is a natural killer (NK) cell.

Aspect 29. The compound of any one of aspects 23-28, wherein the cell at Y¹ or Y² is a T-cell or a B-cell.

Aspect 30. The compound of aspect 29, wherein the T-cell is a CD4⁺ cell, CD8⁺ cell, a helper T cell, a cytotoxic T cell, or a regulatory T cell.

Aspect 31. The compound of any one of aspects 23-26, wherein the cell at Y¹ or Y² is a stem cell.

Aspect 32. The compound of aspect 31, wherein the stem cell is selected from a hematopoietic stem cell, a pluripotent stem cell, and a differentiated stem cell.

Aspect 33. The compound of any one of aspects 1-32, wherein n is 2.

Aspect 34. The compound of any one of aspects 1-32, wherein n is 1.

Aspect 35. A composition, comprising:
a) a target molecule comprising an imidazole of formula (VII):

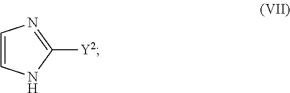

and
b) a biomolecule comprising a phenol moiety or a catechol moiety of formula (I):

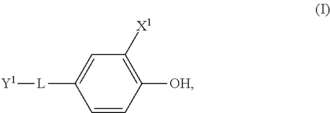

wherein:
Y¹ is a first biomolecule, optionally comprising one or more moieties selected from, an active small molecule, an affinity tag, a fluorophore, and a metal-chelating agent;
X¹ is selected from hydrogen and hydroxyl;
L is an optional linker; and
Y² is a second biomolecule.

Aspect 36. A composition, comprising:
a) a target molecule comprising an amine of formula (VIII):

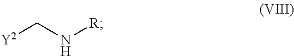

and
b) a biomolecule comprising a phenol moiety or a catechol moiety of formula (I):

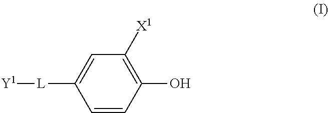

wherein:
Y¹ is a first biomolecule, optionally comprising one or more moieties selected from, an active small molecule, an affinity tag, a fluorophore, and a metal-chelating agent;
X¹ is selected from hydrogen and hydroxyl;
L is an optional linker;
Y² is a second biomolecule; and
R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

Aspect 37. A compound comprising a first biomolecule attached to a second biomolecule, wherein the compound is produced by contacting a second biomolecule in vitro with a first biomolecule comprising a reactive moiety, wherein the second biomolecule comprises on its surface a surface molecule comprising a thiol, an amine, or an imidazole moiety; wherein the first biomolecule comprising the reactive moiety is generated by reaction of a first biomolecule comprising a phenol moiety or a catechol moiety with an enzyme capable of oxidizing the phenol or catechol moiety; and wherein said contacting is under conditions sufficient for conjugation of the surface molecule to the first biomolecule, thereby producing the compound.

Aspect 38. The compound of aspect 37, wherein the second biomolecule is a cell and comprises on its surface receptor a surface molecule comprising a thiol.

Aspect 39. The compound of aspect 37 or 38, wherein the first biomolecule or second biomolecule is a cell.

Aspect 40. The compound of any one of aspects 37-39, wherein the first biomolecule is a cell and the second biomolecule is a cell.

Aspect 41. The compound of any one of aspects 37-39, wherein the first biomolecule or second biomolecule is an antibody.

Aspect 42. The compound of any one of aspects 37, 38, and 41, wherein the first biomolecule is an antibody and the second biomolecule is an antibody.

Aspect 43. The compound of aspect 41 or 42, the antibody binds specifically to a cancer-associated antigen.

Aspect 44. The compound of aspect 43, wherein the cancer-associated antigen is Her-2, CD19, mesothelin, CD20, WT-1, MUC-1, BCMA, claudin-18.2, PD-L1, FLT-3, EGFR, and VEGF.

Aspect 45. The compound of any one of aspects 37-44, wherein the antibody is selected from the group consisting of a single-domain antibody, an IgG1 isotype antibody or fragment thereof, an IgG2 isotype antibody or fragment thereof, an IgG3 isotype antibody or fragment thereof, an IgG4 isotype antibody or fragment thereof, an IgE isotype antibody or fragment thereof, an IgM isotype antibody or fragment thereof, and an Fc domain.

Aspect 46. The compound of any one of aspects 37-45, wherein the antibody is a single-chain Fv or a nanobody.

Aspect 47. The compound of any one of aspects 37-46, wherein the antibody is modified to include a tyrosine residue within 5 amino acids of the C-terminus amino acid of the antibody.

Aspect 48. The compound of any one of aspects 37-47, wherein the antibody is modified to include a C-terminal tyrosine residue.

Aspect 49. The compound of any one of aspects 37-48, wherein the enzyme is a tyrosinase polypeptide.

Aspect 50. The compound of aspect 49, wherein the tyrosinase polypeptide comprises an amino acid sequence having at least 75% amino acid sequence identity to the abTYR amino acid sequence depicted in FIG. 9 or FIG. 10.

Aspect 51. The compound of aspect 49, wherein the tyrosinase polypeptide comprises an amino acid sequence having at least 75% amino acid sequence identity to any one of the amino acid sequences depicted in any one of FIG. 11A-11Z and 11AA-11VV.

Aspect 52. The compound of any one of aspects 37-51, wherein the phenol moiety is present in a tyrosine residue.

Aspect 53. The compound of any one of aspects 37-52, wherein the thiol moiety is present in a cysteine residue.

Aspect 54. The compound of any one of aspects 37-53, wherein the amine moiety is present in a lysine residue.

Aspect 55. The compound of any one of aspects 37-54, wherein the imidazole moiety is present in a histidine residue.

Aspect 56. The compound of any one of aspects 37-55, wherein the first biomolecule comprises one or more moieties selected from a fluorophore, an active small molecule, and an affinity tag.

Aspect 57. The compound of any one of aspects 37-56, wherein the reactive moiety is an orthoquinone or a semi-quinone radical, or a combination thereof.

Aspect 58. The compound of any one of aspects 37-41 and 43-57, wherein the cell is an immune cell.

Aspect 59. The compound of any one of aspects 37-41 and 43-58, wherein the cell is an innate immune cell or an adaptive immune cell.

Aspect 60. The compound of any one of aspects 37-41 and 43-59, wherein the cell is a natural killer (NK) cell, a macrophage, a dendritic cell, a mast cell, an eosinophil, a basophil, a neutrophil, or a monocyte.

Aspect 61. The compound of any one of aspects 37-41 and 43-60, wherein the cell is a T-cell or a B-cell.

Aspect 62. The compound of aspect 61, wherein the T-cell is selected from the group consisting of a CD4⁺ cell, CD8⁺ cell, a helper T cell, a cytotoxic T cell, and a regulatory T cell.

Aspect 63. The compound of any one of aspects 37-41 and 43-59, wherein the cell is a stem cell.

Aspect 64. The compound of aspect 63, wherein the stem cell is selected from a hematopoietic stem cell, a pluripotent stem cell, and a differentiated stem cell.

Aspect 65. A modified cell comprising an antibody attached to a cell, wherein the modified cell is produced by contacting a cell in vitro with an antibody comprising a reactive moiety, wherein the cell comprises on its surface receptor a cell surface molecule comprising a thiol, an amine, or an imidazole moiety, wherein the antibody comprising the reactive moiety is generated by reaction of an antibody modified to include a C-terminal tyrosine residue with a tyrosinase polypeptide, and wherein the contacting is under conditions sufficient for conjugation of the cell surface molecule to the antibody, thereby producing the modified cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

Exemplary phenol and catechol containing intermediates may be synthesized using any convenient method. Methods which can be adapted for use in preparing exemplary phenol and catechol containing intermediates of this disclosure includes those methods described by Maza et al. in “Enzymatic Modification of N-Terminal Proline Residues Using Phenol Derivatives”, J. Am. Chem. Soc. (2019), 141, 3885-3892, the disclosure of which is herein incorporated by reference in its entirety. Many general references providing commonly known chemical synthetic schemes and conditions useful for synthesizing the disclosed compounds are also available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978). Reactions may be monitored by thin layer chromatography (TLC), LC/MS and reaction products characterized by LC/MS and ¹H NMR.

### Example 1

Shown herein is a one-step method for attachment of tyrosine-tagged proteins to cell surfaces. In this approach, a protein of interest is expressed with a C-terminal tyrosine, such as the sequence described above. The protein can be mixed with cells of interest and abTYR for site-selective activation of the introduced tyrosine to the corresponding o-quinone intermediate. These C-terminal o-quinones then react with endogenous nucleophiles present on cell surfaces, producing well-defined points of attachment (FIG. 1B). To demonstrate the applicability of this approach, tyrosine-tagged nanobodies were generated. Nanobodies are small antigen-binders derived from the variable regions of camelid immunoglobulins (FIG. 1C). It is shown herein that abTYR site-specifically oxidizes introduced tyrosine-tags and mediates the attachment of nanobodies to cell surfaces while retaining their antigen-binding abilities. This strategy was extended towards the synthesis of nanobody-natural killer (NK) cell conjugates, which exhibited targeted cell lysis. This strategy thus provides a simple, direct synthetic alternative to genetic engineering for cell-based immunotherapy applications.

FIG. 1A-1C. General strategy for modifying cell surfaces with nanobodics. FIG. 1A. Tyrosine catalyzes the oxidation of small molecule phenols to highly reactive o-quinones, which can modify nucleophiles present on proteins. Engineered tyrosine tags at protein termini can also be oxidized by tyrosinase, producing a site-specific o-quinone on the protein that reacts with protein-based nucleophiles. FIG. 1B. Tyrosine-tagged nanobodies can be site-specifically oxidized by tyrosinase for attachment of these proteins to cells. The resulting linkage produces a well-defined point of attachment for installing nanobodies on cell surfaces while imbuing the target cell with novel antigen-binding functionality. FIG. 1C. Nanobodies are low molecular weight (˜10-15 kDa) antigen-binders derived from the variable region of the camelid antibody (PDB ID 3K1K).

### Materials and Methods

### Abbreviations

abTYR=tyrosinase from Agaricus bisporus
sfGFP=superfolder green fluorescent protein
nbGFP_{Tyr}=GFP-binding nanobody with C-terminal Ser-Gly₄-Tyr tag
nbGFP_{Cys}=GFP-binding nanobody with C-terminal Ser-Gly₄-Cys tag
nbFITC=A76C mutant of nbGFP_{Tyr}, modified with FITC-maleimide
HER2=Human epidermal growth factor receptor 2
nbHER2_{Tyr}=HER2-binding nanobody with C-terminal Ser-Gly₄-Tyr tag
DPBS=Dulbecco's phosphate buffered saline solution without Ca2+ or Mg²⁺.

All reagents were obtained from commercial sources and used without any further purification. Tyrosinase isolated from Agaricus bisporus (abTYR, both 25 kU [SKU=T3824-25KU] and 50 kU [SKU=T3824-50KU] were used in these studies) from Sigma-Aldrich. FITC-5-maleimide, CFSE, MitoTracker Red, Calcein AM dye, and propidium iodide were all purchased from ThermoFisher. FITC-labeled HER2 was purchased from AcroBiosystems. Alkyne-tyramide probe was purchased from MedChemExpress. Spin concentrators with 10 and 100 kDa molecular weight cutoffs (MWCO) and sterile spin filters with 0.22 m pores were purchased from Millipore (Billerica, MA). Doubly distilled water (ddH2O) was obtained from a Millipore purification system.

Liquid chromatography mass spectrometry (LC-MS) analysis. Acetonitrile (Optima grade, 99.9%, Fisher, Waltham, MA), formic acid (1 mL ampules, 99+%, Pierce, Rockford, IL), and water purified to a resistivity of 18.2 MΩ·cm (at 25° C.) using a Milli-Q Gradient ultrapure water purification system (Millipore, Billerica, MA) were used to prepare mobile phase solvents for LC-MS. Electrospray ionization mass spectrometry (ESI-MS) of protein bioconjugates was performed using an Agilent 1260 series liquid chromatograph outfitted with an Agilent 6224 time-of-flight (TOF) LC-MS system (Santa Clara, CA). The LC was equipped with a Proswift RP-4H (monolithic phenyl, 1.0 mm×50 mm, Dionex) analytical column. Solvent A was 99.9% water/0.1% formic acid and solvent B was 99.9% acetonitrile/0.1% formic acid (v/v). For each sample, approximately 15 to 30 picomoles of analyte were injected onto the column. Following sample injection, a 5-100% B elution gradient was run at a flow rate of 0.30 mL/min over 8 min. Data was collected and analyzed by deconvolution of the entire elution profile in order to provide reconsctructed mass spectra that are representative of the entire sample using Agilent MassHunter Qualitative Analysis B.05.00. Percent modification was determined through integration of MS peaks using opensource Chartograph software (www.chartograph.com). The integration of the completely unmodified protein peak served as an internal standard in determining the percent modification.

UV-VIS measurements. A NanoDrop 1000 (Thermo) was used to quantify the samples in this work based on absorbance values at 280 nm (or 488 nm for sfGFP).

Flow cytometry. A ThermoFisher Attune NxT flow cytometer was used for flow analysis. For every dye used, the optimal laser was selected based on the ThermoFisher fluorophore selection guide. Voltage settings were determined using single color and unlabeled samples. For all experiments, 10,000 events were collected on an initial gate in the FSC-A vs SSC-A. The gating scheme employed involved an initial gate on the cell population in the FSC-A vs SSC-A, then a doublet discrimination in the FSC-A vs FSC—H, then a live-dead discrimination in the propidium iodide channel (BL3-H), to produce a histogram of the corresponding sfGFP or FITC signal (BL1-H).

Statistical analyses. For relevant data, a two-tailed T-test was performed to determine statistical significance. A p-value less than 0.001 is denoted with ***, a p-value of less than 0.01 is denoted with **, and a p-value of less than 0.05 is denoted with *.

### Synthetic Procedures

Synthesis of FITC-Labeled nbGFPTyrA77C (nbFITC)

A 1 mL labeling reaction was performed by combining 211 μL of 474 μM nbGFPTyrA77C ([f]=100 μM) with 250 μL of 10 mM FITC-maleimide ([f]=2.5 mM, ThermoFisher, dissolved in 50% DMF:H2O) and diluted to 1 mL via the addition of 539 μL of DPBS. The whole reaction mix was wrapped in tinfoil and rotated end-over-end for 2 h at RT. The reaction was purified using a NAP-10 column (GE Healthcare) and the collected labeled protein was concentrated using a 10 kDa MWCO spin filter (Corning). The reaction was analyzed using an ESI-QTOF mass spectrometer.

Synthesis of nbHER2Tyr-sfGFP Conjugate

Protein-protein conjugation reactions were prepared at a 100 μL scale by combining 12.6 μL of a 478 μM solution of nbHERTy ([f]=60 μM) and 10 μL of a 100 μM solution of sfGFPY200C ([f]=10 μM) with 2.4 μL of a 16.7 μM solution of abTYR ([f]=400 nM) in 55 μL of ddH2O with 20 μL of 100 mM phosphate buffer at pH 6.5 ([f]=20 mM). The reaction was allowed to proceed for 45 min at RT, after which the activity of abTYR was halted via the addition of 2 μL of 100 mM tropolone. To help remove some of the excess nanobody, the reaction was spun four times against 30 kDa MWCO spin filters (Corning) using DPBS, and the reaction was then analyzed using an ESI-QTOF mass spectrometer.

### Protein Cloning and Expression Procedures

Sequences for nbGFP-SG4Y (nbGFPTyr)1 and nbHER2-SG4Y (nbHER2Tyr)2,3 were purchased as gene blocks from International DNA Technologies, Inc. (IDT), designed to have the open reading frame (ORF) inserted into a pET28b(+) expression vector using Gibson assembly. Thus, a pET28b(+) vector was linearized by digestion with XbaI and XhoI for 3 h at 37° C. and purified by column (QIAquick PCR Purification Kit, QIAGEN).

The gene blocks contained 21 and 27 base pairs of homology with the linearized pET28b vector (underlined) that contained the XbaI/XhoI cut sites (bold). The sequences for the nanobodies (UPPERCASE) contained a C-terminal His6 tag for purification (underlined) and an SG4Y handle for tyrosinase chemistry (italicized):

nbGFP-SG4Y (nbGFP_{Tyr}):
(SEQ ID NO: 194)
tagcggataacaattcccctctagaaataattttgtttaactttaagaaggagatataccATGGCAGATGTTCAGC
TGGTTGAAAGCGGTGGTGCACTGGTTCAGCCTGGTGGTAGCCTGCGTCTGAGCTGTGCAGC
AAGCGGTTTTCCGGTTAATCGTTATAGCATGCGTTGGTATCGTCAGGCACCGGGTAAAGAA
CGTGAATGGGTTGCAGGTATGAGCAGTGCCGGTGATCGTAGCAGCTATGAAGATAGCGTTA
AAGGTCGTTTTACCATCAGCCGTGATGATGCACGTAATACCGTTTATCTGCAAATGAATAGC
CTGAAACCGGAAGATACCGCAGTGTATTATTGCAATGTTAACGTGGGCTTTGAATATTGGG
GTCAGGGCACCCAGGTTACCGTTAGCAGCGCAGCAGCACATCATCACCATCATCATTCAGGT
GGTGGTGGTTATTGActcgagcaccaccaccaccaccactgag.

The above nbGFP-SG4Y nucleotide sequence encodes a polypeptide having the following amino acid sequence:

(M)ADVQLVESGGALVQPGGSLRLSCAASGFPVNRYSMRWYRQAPGKEREWVA GMSSAGDRSSYEDSVKGRFTISRDDARNTVYLQMNSLKPEDTAVYYCNVNVGFEYWGQGTQV TVSSAAAHHHHHHSGGGGY (SEQ ID NO:195), where the poly(His) tract is underlined and the SG4Y handle for tyrosinase chemistry (SGGGGY; SEQ ID NO:196) is italicized.

nbHER2-SG4Y (nbHER2_{Tyr}):
(SEQ ID NO: 197)
tgagcggataacaattcccctctagaaataattttgtttaactttaagaaggagatataccATGGAAGTTCAGCTGG
TTGAAAGCGGTGGTGGTCTGGTTCAGGCAGGCGGTAGCCTGCGTCTGAGCTGTGCAGCAAG
CGGTATTACCTTTAGCATTAATACCATGGGTTGGTATCGTCAGGCACCGGGTAAACAGCGTG
AACTGGTTGCACTGATTAGCAGCATTGGTGATACCTATTATGCCGATAGCGTTAAAGGTCGT
TTTACCATTAGCCGTGATAATGCCAAAAATACCGTTTACCTGCAGATGAATAGCCTGAAACC
GGAAGATACCGCAGTGTATTATTGTAAACGTTTTCGTACCGCAGCACAGGGCACCGATTATT
GGGGTCAGGGCACCCAGGTTACCGTTAGCAGCGCAGCAGCACATCATCACCATCATCATTC
AGGTGGTGGTGGTTATTGActcgagcaccaccaccaccaccactgag

The above nbHER2-SG4Y nucleotide sequence encodes a polypeptide having the following amino acid sequence:

(SEQ ID NO: 108)
MEVQLVESGGGLVQAGGSLRLSCAASGITFSINTMGWYRQAPGKQRELV
ALISSIGDTYYADSVKGRFTISRDNAKNTVYLQMNSLKPEDTAVYYCKR
FRTAAQGTDYWGQGTQVTVSSAAAHHHHHHSGGGGY.

With both insert and vector ready for Gibson assembly, 50 ng of linearized vector was combined with insert in a 3:1 insert:vector molar ratio in a volume of 10 uL, and added to 10 μL of 2× Gibson Assembly® Master Mix (New England BioLabs). The resulting mixture was incubated for 1 hr at 50° C. and then transformed into XL1-Blue competent cells via a 45 s, 42° C. heat shock and plated on LB/kanamycin. Resulting colonies were used to inoculate 6 mL LB/kanamycin cultures (50 μg/mL) and grown overnight at 37° C., 225 RPM. Plasmid DNA was isolated using a Qiagen QIAprep Spin Miniprep Kit and sequenced using the pET primer: 5′-CTCGATCCCGCGAAATTA-3′ (SEQ ID NO:198) to confirm insertion of the desired protein sequence.

The nbGFP-SG4C (nbGFPCys) and the nbGFPTyr A76C plasmids were generated using QuikChange II Site-Directed Mutagenesis Kit (Stratagene) using the following primers:

nbGFP-SG4C (nbGFPCys):
fwd primer:
(SEQ ID NO: 201)
5′-ATCATTCAGGTGGTGGTGGTTGTTGActcgagcaccac-3′
rev primer:
(SEQ ID NO: 202)
5′-gtggtgctcgagTCAACAACCACCACCACCTGAATGAT-3′
nbGFPTyrA76C
fwd primer:
(SEQ ID NO: 203)
5′-gcagataaacggtattacgacaatcatcacggctgatggtaaaac
ga-3′
rev primer:
(SEQ ID NO: 204)
5′-tcgttttaccatcagccgtgatgattgtcgtaataccgtttatct
gc-3′

The plasmids were then sequenced using the pET primer to confirm cysteine point mutations (bold and underlined):

nbGFP-SG4C (nbGFPCys):
(SEQ ID NO: 205)
ATGGCAGATGTTCAGCTGGTTGAAAGCGGTGGTGCACTGGTTCAGCCTGGTG
GTAGCCTGCGTCTGAGCTGTGCAGCAAGCGGTTTTCCGGTTAATCGTTATAGCATGCGTTGG
TATCGTCAGGCACCGGGTAAAGAACGTGAATGGGTTGCAGGTATGAGCAGTGCCGGTGATC
GTAGCAGCTATGAAGATAGCGTTAAAGGTCGTTTTACCATCAGCCGTGATGATGCACGTAA
TACCGTTTATCTGCAAATGAATAGCCTGAAACCGGAAGATACCGCAGTGTATTATTGCAAT
GTTAACGTGGGCTTTGAATATTGGGGTCAGGGCACCCAGGTTACCGTTAGCAGCGCAGCAG
CACATCATCACCATCATCATTCAGGTGGTGGTGGTTGT

Which corresponds to the following amino acid sequence:

(SEQ ID NO: 206)
(M)ADVQLVESGGALVQPGGSLRLSCAASGFPVNRYSMRWYRQAPGKEREWVA
GMSSAGDRSSYEDSVKGRFTISRDDARNTVYLQMNSLKPEDTAVYYCNVNVGFEYWGQGTQV
TVSSAAAHHHHHHSGGGGC
nbGFPTyrA76C:
(SEQ ID NO: 207)
ATGGCAGATGTTCAGCTGGTTGAAAGCGGTGGTGCACTGGTTCAGCCTGGTG
GTAGCCTGCGTCTGAGCTGTGCAGCAAGCGGTTTTCCGGTTAATCGTTATAGCATGCGTTGG
TATCGTCAGGCACCGGGTAAAGAACGTGAATGGGTTGCAGGTATGAGCAGTGCCGGTGATC
GTAGCAGCTATGAAGATAGCGTTAAAGGTCGTTTTACCATCAGCCGTGATGATTGTCGTAAT
ACCGTTTATCTGCAAATGAATAGCCTGAAACCGGAAGATACCGCAGTGTATTATTGCAATG
TTAACGTGGGCTTTGAATATTGGGGTCAGGGCACCCAGGTTACCGTTAGCAGCGCAGCAGC
ACATCATCACCATCATCATTCAGGTGGTGGTGGTTAT,

which corresponds to the following amino acid sequence:

(SEQ ID NO: 208)
(M)ADVQLVESGGALVQPGGSLRLSCAASGFPVNRYSMRWYRQAPGKER
EWVAGMSSAGDRSSYEDSVKGRFTISRDDCRNTVYLQMNSLKPEDTAVY
YCNVNVGFEYWGQGTQVTVSSAAAHHHHHHSGGGGY

Sequenced plasmids were then transformed into BL21(DE3)Star competent cells via a 45 s, 42° C. heat shock and plated on LB/kanamycin. Resulting colonies were used to inoculate 11 mL LB/kanamycin cultures (50 g/mL) and grown overnight at 37° C., 225 RPM. A 10 mL portion was then added to 1 L of LB media and grown at 37° C. to an OD600 of 0.6-0.8. Expression was induced with a final concentration of 0.1 mM IPTG, switching the temperature to 18° C. After 18-24 h the cells were collected by centrifugation at 8000 rpm for 15 min at 4° C., then frozen at −80° C. for 20 min. The cells were allowed to thaw slightly and resuspended in 20 mL of Ni-NTA equilibration buffer (20 mM sodium phosphate, 300 mM NaCl, 10 mM imidazole, pH 7.4) with 0.1 mM-1 mM PMSF. They were then sonicated for 6.67 min on ice, with 2 s bursts followed by 4 s rest time (20 min total time per sample) at 85% amplitude. Lysed cells were centrifuged at 12,000×g at 4° C. for 8 min. Next, the supernatant was loaded onto a spin column with 3 mL Ni-NTA resin and rotated for 30 min at 4° C. The resin was washed 10× with 6 mL of Ni-NTA wash buffer (20 mM sodium phosphate, 300 mM NaCl, 25 mM imidazole, pH 7.4), and the protein was eluted 3× with 3 mL Ni-NTA elution buffer (20 mM sodium phosphate, 300 mM NaCl, 250 mM imidazole, pH 7.4). The fractions were collected, buffer-exchanged into PBS, and concentrated using Amicon Ultra 10 kD MWCO centrifugal concentrators (MilliporeSigma).

### Experimental Procedures

In all protocols, DPBS refers to Dulbecco's phosphate buffered saline without Mg2+ or Ca2+. These divalent cations should be avoided as they can interfere with abTYR activity.

### Culturing of NK-92MI Cells

NK-92MI cells were acquired from ATCC and stored on liquid N2 until use. Cells were gently thawed at 37° C. for 1-2 min until no ice was visible in the tube, after which the cells were gently transferred into ˜5 mL of chilled Myelocult H5100 media (StemCell Technologies). The cells were spun at ˜125 g for 5 min and the supernatant removed to remove any cryoprotectant from the cells. The cells were resuspended in ˜4 mL of warm Myelocult H5100 and counted. Cells were diluted down to a final concentration of ˜0.4E6 cells/mL in warm Myclocult H5100 in a T-25 flask (Falcon) and allowed to recover for 3-4 days at 37° C., 5% CO2. NK-92MI cells like to grow in visible clumps and care should be taken not to disturb these clumps. At day 3-4 the NK-92MI cells could be seen visibly clumping, and ˜5 mL of the culture was gently distributed into a fresh T-75 flask (Falcon) prepped with 15 mL of Myelocult H5100 (1:3 passage ratio), being careful not to disturb any of the NK-92MI clumps. The cells were allowed to continue growing for another 3-4 days, after which they typically required another passage. NK-92MI cells were found to be at the appropriate density for downstream experiments at ˜day 3 of the second passage, during which they also showed their maximal activity. NK-92MI cells could be passaged up to 7-8 times, after which their growth and morphology changed and their activity diminished.

### Culturing of Jurkat Cells

Jurkat cells were acquired from ATCC and cultured in RPMI 1640 supplanted with 10% FBS and grown at 37° C., 5% CO2. After thawing and initial recovery in 5 mL of complete media for 3-4 days in a T-25 flask (Falcon), cells were diluted down to a concentration of 0.2E6 cells/mL in either T-75 or T-125 flasks (Falcon). Cells were allowed to reach a maximal density of ˜2E6 cells/mL, typically at day 4 after passage, after which they were diluted back down to 0.2E6 cells/mL in a fresh flask. Jurkat cells could be passaged up to 7-8 times, after which their growth rate changed and their viability diminished.

### Culturing of SK-BR-3 and MDA-MB-468 Cells

Cells were cultured in phenol-red containing DMEM supplanted with 10% FBS and grown at 37° C., 5% CO2 in either T-25 or T-75 flasks (Falcon). Cells were allowed to reach a maximal confluency of ˜80%, typically at day 4 after passage, after which they were trypsinized and passaged at a 1:4 split ratio in complete DMEM.

### Attachment of Nanobodies to Cell-Surfaces

The following general protocol has been found to yield reproducible levels of cell labeling with tyrosine-tagged proteins:

(1) Start by counting cells—each reaction will require ˜1-2 million cells (technical note: if more cells are required in downstream applications prepare additional replicates of each sample, do not scale the reaction beyond 2 million cells). (2) Pool the required number of cells and spin down at ˜300×g for 2 min. (3) Remove supernatant and resuspend cells in ˜1 mL of DPBS. Repeat the centrifugation step and remove the supernatant to wash the cells. Repeat until a total of three washes have been completed. On the final wash, resuspend the cells in 1 mL of DPBS and count the cells. (4) Aliquot the cells so there are ˜1 million cells per tube. Add 1 mL of DPBS to each tube so there is extra volume and perform a final spin down at ˜300 g for 2 min. (5) Remove the supernatant and perform a labeling reaction with 10 μM of tyrosine-tagged nanobody, 400 nM abTYR all in DPBS and at a final volume of 400 μL. (6) Oxidation controls should be prepared in the absence of abTYR, and depending on the downstream application, isotype controls should be prepared using 10 μM of a different tyrosine-tagged nanobody and abTYR. Untreated controls, consisting of just 400 μL of DPBS, should also be prepared. (7) Allow all reactions to proceed for 10 min at 37° C., 5% CO2. After the 10 min, add 1 mL of DPBS to each tube and spin the tube at ˜300×g for 2 min (be sure no FBS or media is present at this step as the abTYR in the reaction will form reactive intermediates with free amino acids and proteins in these mixtures). (8) Wash the cells until a total of two washes have been performed and move on to any downstream applications.

Secondary Labeling of Nanobody-Cell Conjugates with sfGFP

The following general protocol was used to verify successful cell conjugation: (1) Follow the general protocol for the attachment of nanobodies to cell-surfaces as above, being sure to use 10 μM of nbGFPTyr in the reaction. (2) After the 10 min labeling step is done, add 1 mL of DPBS to each tube and spin the tube at ˜300×g for 2 min (be sure no FBS or media is present at this step as the abTYR in the reaction will form reactive intermediates with free amino acids and proteins in these mixtures). (3) Remove the supernatant and resuspend the cells in 1 mL of warm cell binding buffer (5% FBS with 1% w/v of NaN3 all in DPBS). (4) Perform a second spin down and remove the supernatant. Bring each sample up in 200 μL of 1 μM sfGFP in DPBS and allow the samples to sit on ice for 30 min. After 30 min, add 1 mL of cell binding buffer and spin down the cells at ˜300×g for 2 min. (5) Remove the supernatant and repeat the wash steps until a total of three washes have been completed. (6) Resuspend the cells in 1 mL of cell binding buffer supplemented with 2 μL of propridium iodide stock solution (1 mg/mL). (7) Store all cells on ice and analyze using an NxT Attune flow cytometer (ThermoFisher).

### Protocol for Determining Density of Nanobodies on Cell Surface.

Cells were pooled and centrifuged at ˜300×g for 3 min. The supernatant was removed and the cells were rinsed with 1 mL portion of warm DPBS two times. After rinsing, cells were resuspended in 1 mL of DPBS and a cell density measurement was taken. Approximately 1E6 cells were transferred to each tube and then diluted with 1 mL of DPBS. A final spin down at ˜300×g for 2 min was performed and the supernatant was removed. Labeling reactions were performed using 10 μM, 5 M, or 1 μM final concentrations of nbFITC with 400 nM abTYR in 400 μL of DPBS. A control reaction was run using 10 μM final concentration of nbFITC with no abTYR in 400 μL DPBS, as well as an untreated control consisting of just 400 μL of DPBS. The labeling reactions were allowed to proceed for 10 min at 37° C., 5% CO2. After the reaction was complete, the reactions were diluted with 1 mL of warm DPBS and centrifuged at ˜300×g for 2 min. The supernatant was removed and the cells were rinsed with 1 mL of warm cell binding buffer (DPBS supplanted with 5% FBS and 1% NaN3 w/v) two times. The cells were resuspended in 1 mL of warm cell binding buffer with the 2 μL of propridium iodide stock solution (1 mg/mL). All cells were stored on ice and analyzed using an NxT Attune flow cytometer (ThermoFisher). For FITC density measurements, the protocol from the Quantum FITC-5 MESF kit from Bangs Laboratories was used.

### Protocol for Imaging Subcellular Location of Nanobody Attachment

Cells were modified with 10 μM nbFITC in the presence or absence of 400 nM abTYR according to the above protocol. After attachment, cells were washed two times with 1 mL portions of cell binding buffer (5% FBS with 1% w/v NaN3 in DPBS) and then resuspended in 1 mL of cell binding buffer. To this was added 1 μL of CellMask Deep Red (ThermoFisher) cell membrane dye stock solution, and the cells were stained for 10 mins at 37° C. After cell membrane staining, cells were washed two times with 1 mL portions of cell binding buffer and then fixed with 1 mL of 4% PFA (v/v) in DPBS for 10 min at RT. After fixing, cells were imaged using a Zeiss LSM880 confocal microscope.

### Proteomics Experiment

Jurkat cells were grown to a density of 2e6 cells/mL in T-125 flasks (Falcon) in ˜50-100 mL of complete RPMI 1640 media and then ˜100 million Jurkat cells were pelleted at 300 g for 5 min. Cell pellets were washed with 50 mL of DPBS two times. Jurkat cells were labeled in a 10 mL reaction in pre-warmed DPBS with alkynyl-tyramide probe (+100 μL of 10 mM alkynyl-tyramide probe, [f]=10 μM) and abTYR (+240 μL of a 16.7 μM abTYR stock [˜2 mg/mL], [f]=400 nM). The labeling reaction was allowed to proceed for 20 min at 37° C. with shaking at 220 rpm. After 20 min, the reaction was diluted with 40 mL of DPBS and pelleted at 300 g for 5 min. The supernatant was removed and the pellet was washed two more times with 50 mL of DPBS. Cells were resuspended in 1 mL of RIPA lysis buffer (ThermoFisher) supplemented with protease inhibitor cocktail (ThermoFisher). Cells were lysed using probe ultrasonication (20% amplitude, 10 cycles of 5 sec on/1 sec off). The probe-labeled proteome sample was diluted to a final concentration of 2 mg/mL of protein in solution in 2 mL of DPBS. The diluted proteome sample was then aliquoted at 500 μL volumes into four separate 2 mL eppendorf tubes. To each tube was then added 10 μL of a 5 mM N3-TEV-biotin tag (9.24 mg/mL). To this mix was then added 10 μL of freshly prepared 50 mM TCEP solution (in water) as well as 30 μL of TBTA ligand solution (0.9 mg/mL in DMSO:t-butanol=1:4) and the mixture was vortexed. To this was added 10 μL of 50 mM Cu(II) Sulfate (12.5 mg/mL in water) and the sample was vortexed. The reaction was incubated at RT for 1.5 hr with vortexing every 15 min (note: at this stage the proteins will start to precipitate and the reaction will grow cloudy). After the reaction, the tubes were centrifuged at 6500 g for 4 min. The supernatant was removed from the pelleted proteins, and to the pellet in each tube was added 500 μL of pre-chilled MeOH. The pellets were then resuspended using probe ultrasonication (20% amplitude for 10 sec). All four resuspended pellets were then combined into a single 2 mL eppendorf tube, and the combined samples were centrifuged at 6500 g for 4 min at 4° C. The supernatant was removed, and the combined pellet was washed two more times with 500 μL of pre-chilled MeOH. After the last wash step, the supernatant was removed and the pellet was resuspended in 1 mL of 1.2% SDS/PBS (w/v) and sonicated for several seconds until the solution turned clear. The resuspended pellet was then heated at 80-90° C. for 5 min and then centrifuged at 6500 g for 5 min. Meanwhile, streptavidin agarose beads were washed with 1 mL portions of PBS three times and then resuspended in their original volume with PBS. Using a 200 μL pipette tip with its end cut off, 170 μL of washed heads were transferred to a 15 mL conical containing 5 mL of PBS. The supernatant was then removed from the centrifuged protcomic sample and applied to the 15 mL conical containing the beads ([f] of SDS in the sample is 0.2%). The labeled proteome sample rotated overnight at 4° C. and was allowed to bind to the streptavidin resin.

After incubating overnight, the proteome samples were rotated for 2-4 hr at RT to resolubilize the SDS. The conical was then centrifuged at 1400 g for 3 min and the supernatant was removed. The beads were washed via the addition of 5 mL of 0.2% SDS/PBS (w/v), placed on a rotator for 10 min, and then spun at 1400 g for 3 min and the supernatant removed. The beads were transferred to Micro Bio-Spin columns using two washes of 500 μL of PBS and washed on a vacuum manifold using three 1 mL portions of PBS. The beads were then washed with three 1 mL portions of water. The beads were transferred to eppendorf tubes using two 250 μL washes of freshly prepared 6 μM urea/PBS (1.8 g/5 mL). To the tube was added 25 μL of freshly prepared DTT solution (30 mg/mL in water) and the tube was incubated at 65° C. for 20 min. The tube was then allowed to cool to RT before adding 25 μL of freshly prepared 400 mM iodoacetamide solution (74 mg/mL in water). The tube was incubated with agitation at 37° C. for 30 min. The reaction was diluted by adding 950 μL of PBS and then spun at 1400 g for 2 min to remove the supernatant. To the tube was added a premixed solution of 200 μL of 2 μM urea/PBS, 2 μL of 100 mM calcium chloride in water, and 4 μL of trypsin solution (20 g of lyophilized trypsin in 40 μL of trypsin buffer).

The tube was allowed to incubate overnight in a shaking incubator at 37° C., being sure that the reaction did not proceed for longer than 21 hr.

The next day, the beads were transferred to a Bio-Spin column and the supernatant was removed. The heads were washed with three portions of 500 μL PBS followed by three portions of 500 μL of water. The beads were then transferred to a new eppendorf tube using 2 portions of 500 μL of water and spun down at 1400 g for 2 min. The beads were then washed with 1×TEV buffer (141 μL water, 7.5 μL of 20×TEV buffer, 1.5 μL of 100 mM DTT) and spun down again. The supernatant was removed, and to the beads was added the premixed solution of TEV buffer as above, this time containing L of Ac-TEV protease. The TEV elution step was allowed to proceed for at least 24 hr at 29° C. with mild agitation, after which the beads were transferred to a new Bio-Spin column and the released peptides were eluted into a new eppendorf tube via centrifugation at 1400 g for 2 min. The beads were washed with two portions of 75 μL of water and the washes were combined with the initial eluent. To the eluted peptide sample was added 16 μL of LC-MS grade formic acid, and the samples were stored at ˜80° C. until mass spectrometric analysis.

Data were extracted in the form of MS1 and MS2 files using Raw Extractor v.1.9.9.2 (Scripps Research Institute) and searched against the Uniprot human database using ProLuCID search methodology in IP2 v.3 (Integrated Proteomics Applications, Inc.). Cysteine residues were searched with a static modification for carboxyaminomethylation (+57.02146) and the TEV tag probe modification +615.3129. Lysine residues were searched for the TEV tag probe modification +613.29724. Histidine residues were searched for the TEV tag probe modification +613.29724. Peptides were required to be fully tryptic peptides and to contain the TEV modification. ProLUCID data were filtered through DTASelect to achieve a peptide false-positive rate below 5%. After filtering assigning modifications, all peptides were filtered based on the presence of the TEV tag probe modification and any duplicates were removed and the sum of observed cysteine, lysine, and histidine modifications were extracted.

Experiment for Evaluating nbHER2Tyr-sfGFP Conjugate Binding Specificity

T-25 flasks (Falcon) were grown to 80% confluency containing either the HER2⁺ cell line SK-BR-3 or the HER2− cell line MDA-MB-468. Cells were trypsinized and resuspended in 1 mL of warm cell binding buffer (DPBS supplanted with 5% FBS and 1% NaN3 w/v). Cells were spun down at ˜300×g for 2 min and the supernatant removed, then washed with 1 mL portions of warm cell binding buffer an additional two times. Cells were brought to a final concentration of 2.0E6 cells/mL in warm cell binding buffer. To a 100 μL aliquot of either the SK-BR-3 or MDA-MB-468 cells was added 20 μL of 10 μM nbHER2Tyr-sfGFP ([f]=2 nM). As negative controls, 10 μL of DPBS were added to separate 100 μL aliquots of the cells. Samples were allowed to incubate on ice for 1 h, were diluted with 1 mL of warm cell binding buffer and spun down at ˜300×g for 2 min. The supernatant was removed and samples were then washed an additional two times with 1 mL of warm cell binding buffer for a total of three washes. Finally, samples were resuspended in 1 mL of warm cell binding buffer and placed on ice until analysis via flow cytometry.

### Cell-Cell Binding Study

To begin, the HER2⁺ cell line SK-BR-3 was loaded with CFSE dye (ThermoFisher). A T-75 flask with SK-BR-3 cells grown to 80% confluency was lifted using 1 mL of trypsin solution at 37° C., 5% CO2 for 90 see. Once the cells were free, the trypsin was diluted with 9 mL of warm complete DMEM. The cell suspension was collected and spun at ˜300×g for 3 min. The supernatant was carefully removed and the cells were rinsed in 1 mL of warm DPBS three times. The cells were resuspended in 1 mL of warm DPBS and a cell density measurement was measurement. To the cells in 1 mL of DPBS was added 0.5 μL of CFSE stock solution (10 mM) per 1 million SK-BR-3 cells. The cells were allowed to incubate at 37° C., 5% CO2 for 10 min. After the incubation was complete, the cells were centrifuged at ˜300×g for 3 min, the supernatant was removed, and the cells were rinsed three times with 1 mL of portion of warm, complete DMEM (phenol-red free). After rinsing, the CFSE-labeled SK-BR-3 cells were diluted to 40,000 cells/mL and stored on ice until use.

Next, the NK-92MI cells were pooled and spun down at ˜300×g for 3 min. The supernatant was removed and the cells were rinsed with 1 mL portion of warm DPBS for a total of two washes. After the washes, the cells were counted and ˜1-2 million NK cells were aliquoted into four tubes for labeling reactions. Labeling reactions were performed with 10 μM nbHERTyr and 400 nM abTYR in 400 μL of DPBS. A control was performed in the absence of abTYR, as well as an isotype control which involved labeling the cells with 10 μM nbGFPTyr and 400 nM abTYR in 400 μL of DPBS. A no treatment control was also prepared, which just involved diluting the cells in 400 μL of DPBS only. Labeling reactions were allowed to proceed for 10 min at 37° C., 5% CO2, after which the cells were diluted with 1 mL of warm DPBS and spun down at ˜300×g for 2 min. Cell were rinsed with 1 mL portions of DPBS two times and then resuspended in 1 mL of warm DPBS. Cell density measurements were performed, and 0.5 μL of a Mitotracker Red stock solution (1 mM) were added per 1 million cells to each tube. The cells were incubated at 37° C., 5% CO2 for 10 min and then rinsed three times with 1 mL of warm, complete DMEM (phenol-red free). The samples were brought up in 0.5 mL of warm, complete DMEM (phenol-red free) and brought to a density of 80,000 cells/mL in this same medium.

To a black-walled 24 well plate (Vision Plate, Brooks Life Sciences) was added 0.5 mL of the SK-BR-3 cell solution (at 40,000 cells/mL) and 0.5 mL of the labeled NK cell solution (at 80,000 cells/mL) for a final ratio of 1:2 SK-BR-3 to NK cells. Cell-cell binding samples were prepared in quadruplicate and the cells were allowed to sit at 37° C., 5% CO2 for 2 h. The cells were then imaged using a high throughput fluorescence microscope (ImageXpress Micro, Molecular Devices), collected 25 unique regions from every well. A nearest neighbor analysis between the CFSE-labeled SK-BR-3 cells and the MitoTracker Red-labeled NK cells was performed in CellProfiler (Broad Institute) and the results were exported into Excel (Microsoft).

A SK-BR-3 cell was marked as “bound” if it was found to be neighbored by 2 or more NK cells. The number of “bound” SK-BR-3 cells was then divided by the total number of SK-BR-3 cells detected to determine the proportion of SK-BR-3 cells bound by NK cells in each condition.

General protocol for cell killing study. (1) Start by loading the HER2⁺ cell line SK-BR-3 with calcein AM dye (ThermoFisher). Lift a T-75 flask with SK-BR-3 cells grown to 80% confluency with 1 mL of trypsin solution at 37° C., 5% CO2 for 90 s. Once the cells are free, dilute the trypsin with 9 mL of warm complete DMEM. Collect the cell suspension and spin at ˜300×g for 3 min. Carefully aspirate off the supernatant and resuspend the cells in 1 mL of warm complete DMEM. (2) Take a cell count measurement, and for every million SK-BR-3 cells add 4 μL of a 1 mg/mL calcein AM stock solution (prepared according to ThermoFisher specifications). Incubate the cells for ˜30 min at 37° C., 5% CO2. While the cells are labeling, move on to the abTYR-mediated synthesis of the nanbody-NK cell conjugates, detailed below—it is okay if the calcein AM incubation goes over 30 min while the NK cells are being labeled. (3) After the SK-BR-3 cells are done labeling with calcein AM, spin down the cells at ˜300×g for 2 mins. Rinse the cells twice with 1 mL of warm DPBS, and after the second wash resuspend the cells in warm Myelocult H5100. (4) Keep the cells off to the side until ready to mix with NK cells. (5) While the SK-BR-3 cells are incubating with the calcein AM dye, move on to labeling the NK-92MI cells. Pool all the NK-92MI cells and spin down at ˜300×g for 3 min. Remove the supernatant and wash the cells twice with warm DPBS. After the second wash, resuspend the cells in 1 mL of warm DPBS and take a cell count measurement. (6) Aliquot out ˜1 million NK-92MI cells into separate tubes (n.b. if more labeled NK-92MI cells are required in downstream applications set up multiple reaction tubes in parallel as opposed to scaling up the labeling reaction). Rinse the aliquoted NK-92MI cells one final time with 1 mL of warm DPBS and remove the supernatant. (7) Prepare labeling reactions at 400 μL volume in DPBS with 10 μM nbHERTyr and 400 nM abTYR, as detailed above. Set up a negative control in the absence of abTYR and an isotype control using 10 μM nbGFPTyr and 400 nM abTYR, as well as a untreated controls consisting of 400 μL of warm DPBS. Allow the reactions to incubate for 10 min at 37° C., 5% CO2. (8) After incubation, add 1 mL of warm DPBS and spin down the reactions at ˜300×g for 2 min. Remove the supernatant and rinse the cells with 1 mL portions of DPBS another two times (n.b. at this point any replicates of a particular condition can be combined into a single tube for washing). (9) After washing, bring the cells up in 1 mL of warm Myelocult H5100 and take cell density measurements. (10) Once all the cells are labeled, combine the different NK samples with the calcein-AM labeled SK-BR-3 cells in a 96 well plate at 100 μL volumes containing 5,000 SK-BR-3 cells and the desired ratio of NK cells (i.e. for a 5-fold excess of NK cells use 25,000 NK cells per well). To do so, prepare master mixes of each sample containing 250,000 labeled NK cells and 50,000 labeled SK-BR-3 cells all in 1 mL. Immediately after preparing a particular master mix sample, aliquot out 100 μL of the mixed cells into a black walled 96-well plate (Corning). (11) Aliquot out all the samples in at least quadruplicate. These constitute the experimental samples. Be sure to include at least eight replicates of Myelocult H5100 media only samples and SK-BR-3 only samples. These will serve, respectively, as a media correction control and a spontaneous dye release correction control. (12) Allow the whole plate to sit at 37° C., 5% CO2 for 4 h. (13) Forty-five min before the endpoint of the experiment, add 10 μL of Lysis solution (Promega) to half of the Myelocult H5100 media only samples and SK-BR-3 only samples. These will serve, respectively, as a volume correction control and a maximum dye release control. (14) At 4 h, spin down the plate of cells at ˜300×g for 3 min, then collect 50 μL of supernatant from all wells and transfer to a new black-walled 96 well plate, being sure not to transfer any cell material at the bottom of the wells. (15) Analyze the supernatant in a plate reader (Tecan), exciting at 490 nm and reading fluorescence at 530 nm from the bottom. (16) With the fluorescence readings in hand, first perform a background correction using the average fluorescence values from the Myelocult H5100 only samples for all samples except the lysed SK-BR-3 cells, which should be corrected using the average fluorescence values from the Myelocult H5100 plus lysis buffer samples. (17) To determine percent specific cytotoxicity of each sample, use the following equation:

$$\% Specific Cell Lysis = \frac{[ Experimental - Spontaneous Release ]}{[ Maximal Release - Spontaneous Release ]} * 100.$$

### Results

### Tyrosinase-Mediated Synthesis of Nanobody-Cell Conjugates

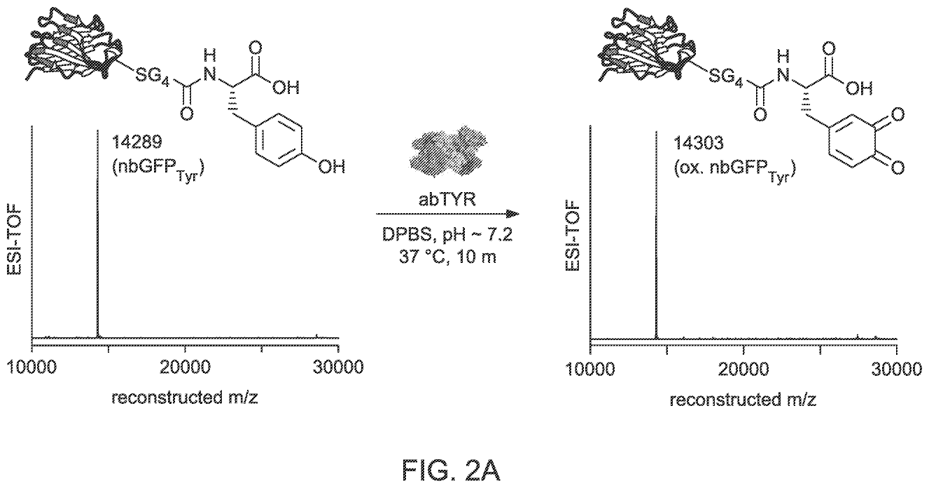

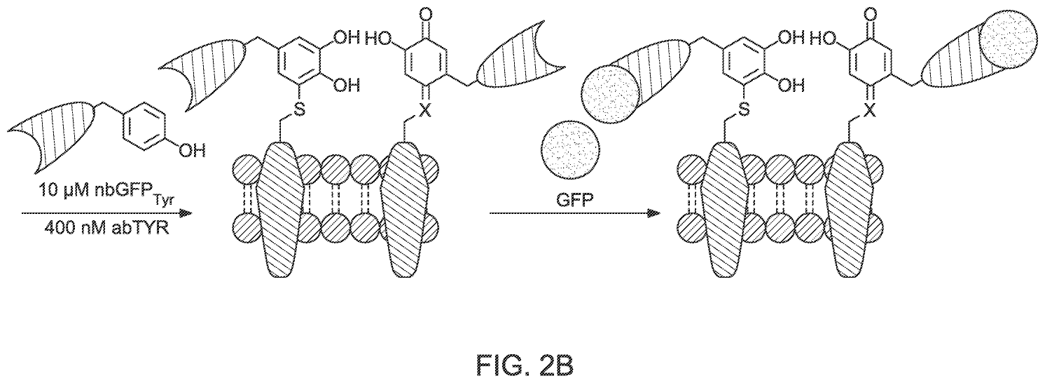

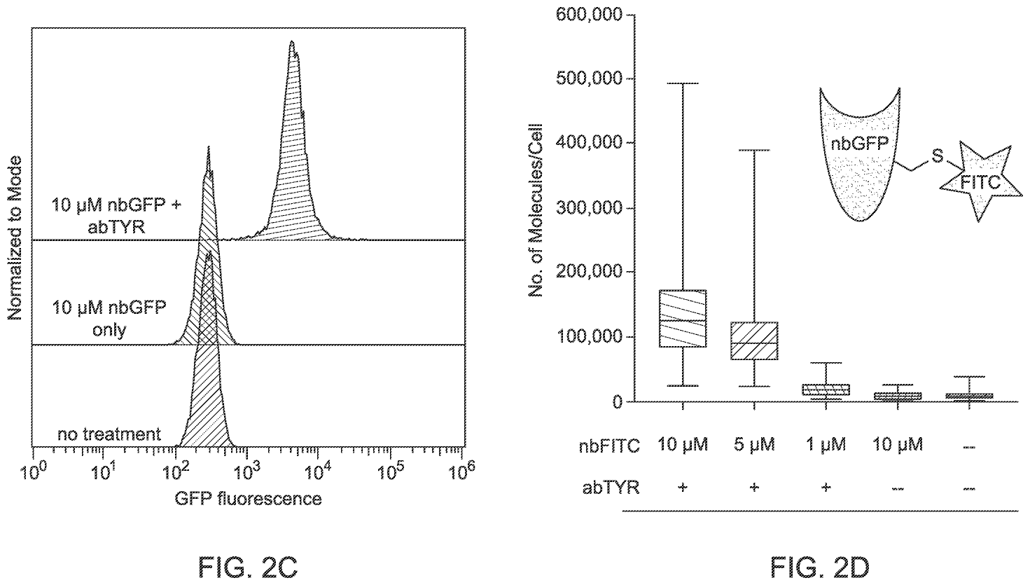

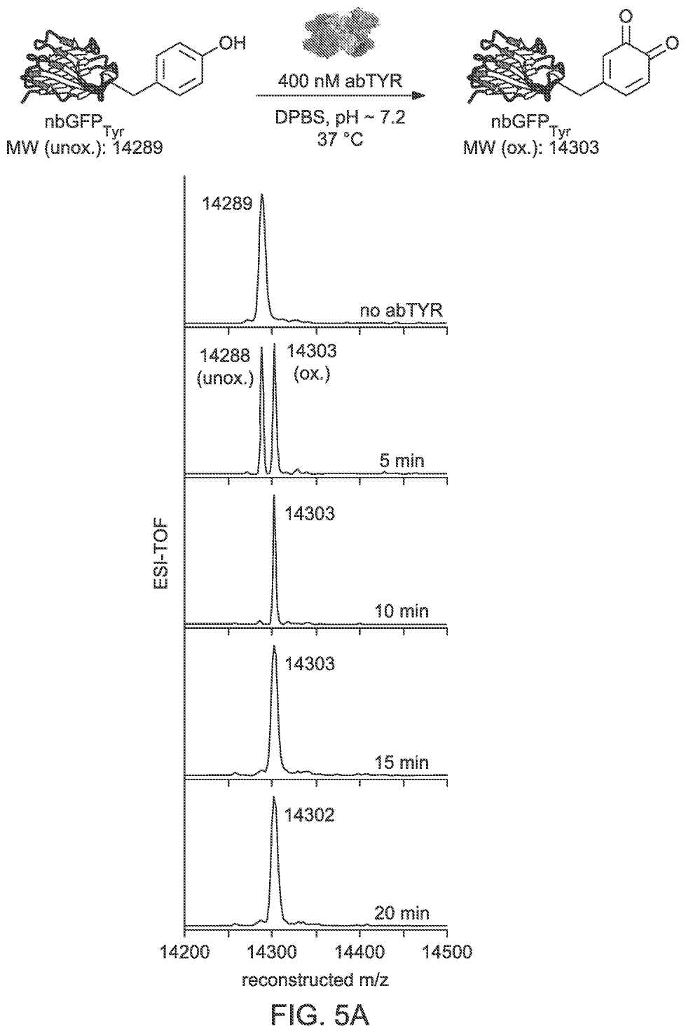

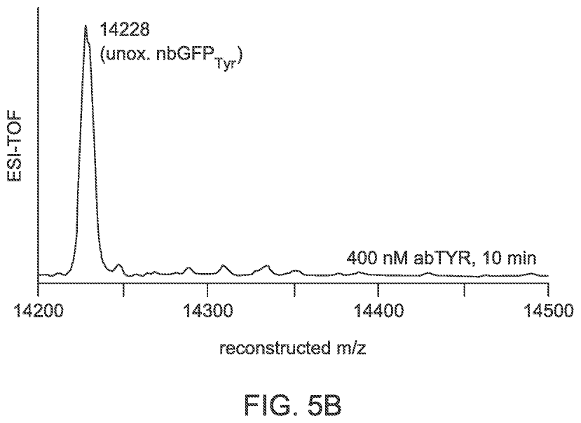

A nanobody against green fluorescent protein (GFP) was expressed with a C-terminal Ser-Gly₄-Tyr tag (nbGFP_{Tyr}). To test the ability of abTYR to selectively oxidize the introduced tyrosine tag, 10 μM nbGFP_{Tyr} was exposed to 400 nM abTYR at 37° C. A time-course experiment was performed, and analysis using ESI-TOF mass spectrometry revealed that the nbGFP_{Tyr} was fully converted to a singly-oxidized product after 10 mins, as indicated by a mass shift of ˜14 Da (FIG. 2A, FIG. 5). Importantly, if the tyrosine residue in the tyrosine tag was swapped for a cysteine (nbGFP_{Cys}) no change in molecular weight was observed in the ESI-TOF MS trace, and no disulfide was formed. Taken together, these results indicate that it is the single phenol in the C-terminal tyrosine-tag being oxidized by abTYR, providing a uniquely reactive o-quinone on the nanobody for attachment to cell surfaces (FIG. 5).

FIG. 2A-2D. Modification of NK cell surfaces with nanobodies. FIG. 2A. The tyrosinase enzyme produces a site-specific o-quinone at the C-terminal Ser-(Gly)₄-Tyr tag installed on nanobodies, as evidenced by a 14 dA mass shift detected via ESI-TOF MS. FIG. 2B. To verify that NK cell surfaces can be decorated with nanobodies using tyrosinase, a Tyr-tagged nanobody against GFP (nbGFP_{Tyr}) was designed. Using tyrosinase, nbGFP_{Tyr} can be attached to the cell surface and secondary labeling with GFP can be used to analyze the reaction using flow cytometry. FIG. 2C. Labeling experiments with nbGFP_{1yr} validated attachment of the nanobody to the cell surface, as only cells treated with both nbGFP_{Tyr} and tyrosinase showed an increase in GFP fluorescence (red trace) over controls (blue and oranges traces). FIG. 2D. Using a Cys point mutant, a single FITC dye can be attached to each nbGFP_{T}yr (nbFITC). After attachment of 10 μM nbFITC to cell surfaces, comparison against FITC calibration beads determined that a median value of 120,000 copies of the nanobody were linked to the cells.

FIG. 5A-5B. Nanobody oxidation tests. FIG. 5A. To determine the optimal amount of time required for tyr-tagged nanobody oxidation, a time-course experiment was performed on 10 μM nbGFP_{1yr} with 400 nM abTYR in DPBS at 37 C. Oxidation of the parent nanobody (MW=14289) was complete by 10 min, as indicated by a MW shift of ˜14 kDa via ESI-QTOF (MW=14303). FIG. 5B. As a control, nbGFP_{Cys}, which has a C-terminal Ser-Gly₄-Cys tag as opposed to a Ser-Gly₄-Tyr tag, was subjected to the same conditions. After 10 minutes, no change in mass was observed (MW=14228), indicating that abTYR was acting on the installed tyrosine residue in the C-terminal tyr-tag.

The ability of abTYR to catalyze the attachment of nbGFP_{Tyr} to cell surfaces was explored. As a model cell system, NK cells, which have recently emerged as promising agents for targeted immunotherapies, were used. Like T-cells, NK cells possess cytotoxic effector functions, and studies have shown that adoptively transferred NK cells are less prone to host rejection than their T-cell counterparts. Additionally, methods for generating a robust supply of immortalized NK cells have been developed, enabling the scalable synthesis of engineered NK variants. NK-92MI is an immortalized NK cell line that constitutively expresses the gene for hTL-2, a cytokine required for NK cell proliferation and activation, facilitating cell culture and analysis of downstream effector functions. However, these cells do not express Fe receptors, limiting their use in antibody-dependent cell cytotoxicity applications. Thus, the direct modification of this cell type with novel antigen-binding functionalities would provide exciting avenues for the synthesis of “off-the-shelf” NK-based immunotherapeutics.

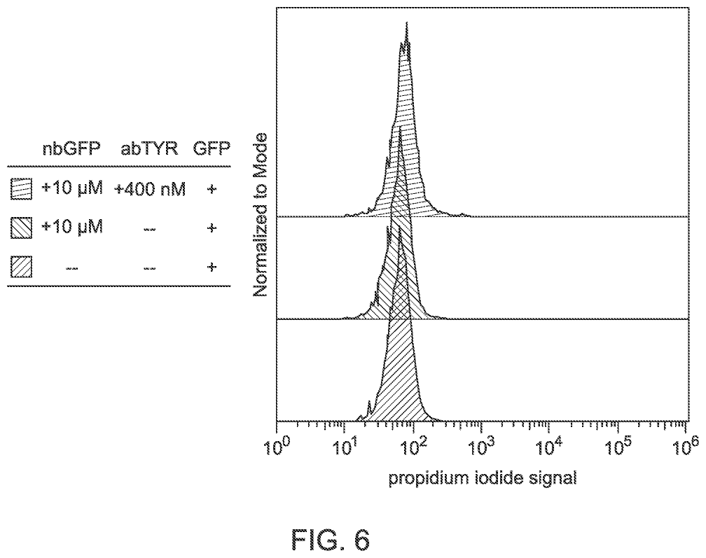

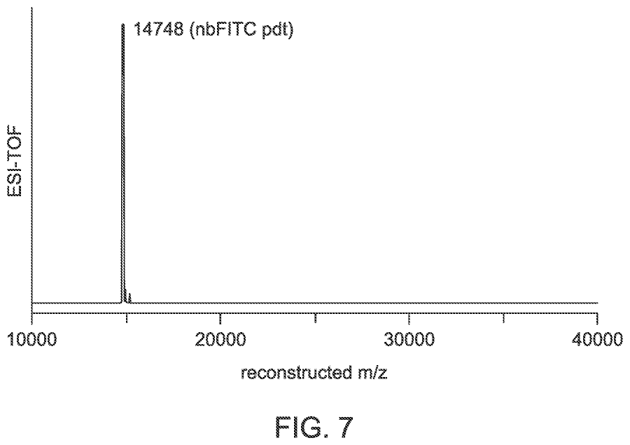

As an initial proof of concept, ˜1×10⁶ NK-92MI cells were exposed to 10 μM nbGFP_{Tyr} with 400 nM abTYR for 10 mins at 37° C. in a cell incubator under 5% CO₂. To validate successful attachment of the nbGFP_{1yr} to the cell surfaces, cells were washed and subjected to a secondary labeling step with 1 μM sfGFP for 30 mins (FIG. 2B). After washing away any unbound sfGFP, cells were analyzed for GFP fluorescence using flow cytometry. An increase in GFP fluorescence was only detected in the cells that had been treated with both nbGFP_{Tyr} and abTYR (FIG. 2C, red trace). Cells exposed to only nbGFP_{Tyr} showed no change in fluorescence over no treatment controls (FIG. 2C, blue and orange traces). These results indicate that the oxidation of nbGFP_{Tyrr} by abTYR facilitates covalent attachment of the nanobody to the cell surface, and that the attached nanobody retains antigen binding. Importantly, cell viability was unchanged after modification, as no increase in propidium iodide signal, a fluorescent indicator of cell death, was detected for those NK cells modified with nanobodies over controls (FIG. 6). In addition, half-life studies were also performed. It was determined that the attached nanobodies were retained on the cell surfaces with a half-life of ˜7.8 h (FIG. 7). This is comparable to other techniques that covalently attach proteins to the cell surface, and means NK cell surfaces should be returned to their unmodified state within 48 h.

FIG. 6. Evaluation of NK-92MI cell viability following modification with nanobodies and abTYR. No increase in propidium iodide signal (a live-dead stain) was detected via flow cytometry after cells were modified with nbGFP_{Tyr} and abTYR (red trace) over negative controls (blue and orange traces) indicating no change in viability following the reaction.

FIG. 7. Synthesis of a FITC-labeled nanobody. The nbGFP_{Tyr}A76C mutant (MW=14321) was modified with FITC-maleimide following the protocol described above. The modification went to completion, as indicated by the shift of the nanobody MW to 14748 (nbFITC) by ESI-QTOF.

To determine the number of nanobodies that were attached to the cell surface, a nbGFP_{Tyr} bearing an alanine to cysteine mutation at position 76 was generated. A maleimide-FITC dye was then site-specifically attached to this cysteine (nbFITC, FIG. 8A-8C). Treatment of cells with this construct and abTYR resulted in attachment of nbFITC to the cell surface. Since a single FITC molecule is on every nanobody, comparison of the nbFITC-labeled cells to FITC calibrant beads, which contain a known number of FITC molecules, enabled the determination of the number of nanobodies on each cell surface. Using this approach, it was determined that a median of ˜120,000 copies of the nanobody were attached to each NK-92MI cell surface upon exposure to 10 μM nanobody and 400 nM abTYR for 10 mins. The amount of nbFITC deposited on the cell surface can be tuned, as lower concentrations of the nanobody resulted in less modification. However, even nanobody concentrations as low as 1 μM resulted in fluorescence labeling above background levels (FIG. 2D).

FIG. 8A-8C. Synthesis of nanobody-Jurkat cell conjugates. FIG. 8A. To test if abTYR can mediate the attachment of nanobodies to the surface of Jurkat cells, ˜1 million Jurkat cells were modified with 10 μM of nbFITC and 40 nM abTYR (red trace). After 10 minutes at 37C, 5% CO₂, the cells showed an increase in FITC fluorescence via flow cytometry over no abTYR and untreated controls (blu and orange traces). FIG. 8B. Following nanobody attachment, Jurkat cells were also evaluated for changes in cell viability. No increased in propidium iodide signal was observed for cells treated with nbFITC and abTYR over controls, indicating no change in cell viability following modification. FIG. 8C. After attachment of 10 μM nbFITC to the Jurkat cell surfaces using tyrosinase, comparison against FITC calibration beads determined that a median value of ˜210,000 copies of the nanobodies were linked to the cells.

As a comparison, labeling reactions were performed on Jurkat cells, an immortalized T-cell line. Approximately ˜1×10⁶ Jurkat cells were incubated with varying concentrations of nbFITC in the presence or absence of 400 nM abTYR. After incubation for 10 mins at 37° C., cells were washed and analyzed for FITC fluorescence using flow cytometry. Jurkat cells exposed to both nbFITC and abTYR showed an increase in FITC signal over nbFITC only and untreated controls (FIG. 9). Once again, cell viability remained unchanged after modification, as no increase in propidium iodide signal was observed (FIG. 9). Comparison against FITC-calibration beads showed that using 10 μM of nbFITC and 400 nM abTYR attached a median of ˜210,000 copies of the nanobodies to the cell surface (FIG. 9). Nanobody attachment could be tuned, with concentrations of nanobody as low as 1 μM resulting in fluorescence labeling over controls.

FIG. 9. Imaging of subcellular location of abTYR-mediated nbFITC attachment. Jurkat cells were labeled with 10 μM nbFITC in the presence or absence of 400 nM abTYR. Cell membranes were then labeled with CellMask Deep Red (ThermoFisher). Imaging with a confocal microscope revealed distinct halos of FITC signal only in cells treated with nbFITC and abTYR (top images, nbFITC and merge images). Some nonspecific binding of nbFITC was observed in the absence of abTYR (middle images), but no clear halo was observed.

Human PBMCs were also modified using this same approach. Similar increases in fluorescence were observed only when PBMCs were exposed to both 10 μM nbGFP_{Tyr} and 400 nM abTYR, while retaining high cell viability.

To validate that nanobody attachment was occurring at the cell surface, Jurkat cells were modified with 10 μM nbFITC in the presence or absence of 400 nM abTYR. After reaction, the cell membrane was then labeled with a cell membrane specific far-red dye. Imaging using a confocal microscope revealed distinct halos of FITC signal only in the cells treated with nbFITC and abTYR, which colocalized with the cell membrane dye. Some nonspecific binding of nbFITC was observed in cells treated with nbFITC in the absence of abTYR, but no clear halo was observed.

Jurkat cells are a robust cell line and can be easily cultured to produce large quantities of cells. As a result, they are a useful cell line for proteomics experiments which often require large amounts of cellular material. To understand the nature of the site of attachment between the cell surface and tyrosine-tagged nanobodies, a proteomics experiment was designed, using an alkynyl-tyramide handle. This small molecule probe has a phenol-moiety for abTYR activation and attachment to cellular proteins as well as a terminal-alkyne moiety for reaction with azide-bearing molecules in a Cu(II)-mediated azido-alkyne cycloaddition. To ˜100×10⁶ Jurkat cells was added 100 μM alkynyl-tyramide probe and 400 nM abTYR to a final volume of 10 mL. Cells were incubated at 37° C. for 20 mins and then washed. After washing, cells were lysed, and modified proteins were labeled with a N₃-TEV-biotin peptide tag. Proteins were then bound to a streptavidin-agarose resin and trypsin digested. Alkynyl-tyramide probe-modified peptides were released using TEV protease and then analyzed using mass spectrometry.

Proteomic analysis identified 437 unique peptide sequences modified with the alkynyl-tyramide probe. Analysis of the amino acid residues modified with the probe identified lysine (50.6% of modified residues), histidine (30.2% of modified residues), and cysteine (19.2% of modified residues) as the nucleophilic sites responsible for modification. While modification of thiol groups with abTYR-generated o-quinones has already been established, this is one of the first examples of o-quinones reacting with the amines and imidazoles of lysine and histidine. In purified protein labeling reactions, off-target modification with lysine and histidine residues has not been observed, even when the protein being modified contains a highly solvent accessible His₆-tag for purification.

The uniquely identified proteins were analyzed, based on their annotated subcellular localization. It was found that only ˜23% of the identified proteins represented known cell membrane or secreted proteins, with the rest of the annotations representing proteins inside the cell, suggesting that this small molecule probe is capable of crossing the cell membrane before it quenches.

To explore if the small molecule proteomics data were representative of the modifications using tyrosine-tagged proteins, a proteomics experiment was performed using a biotinylated nbGFP_{Tyr} as bait (nbBAIT). After labeling the cell surface with this nanobody, the cells were lysed and any protein modified by the biotinylated nbBAIT protein was captured using a streptavidin-agarose resin. The proteins were digested using trypsin and the resulting peptides were submitted for proteomic analysis. Of the proteins captured in this experiment, only nine overlapped with the small molecule proteomics experiment. Seven of these proteins have a known extracellular annotation and represent unique peptides in our small molecule experiment that showed cysteine, lysine, and histidine modifications.

Targeted Cell-Cell Interactions in abTYR Synthesized Nanobody-NK Cell Conjugates.

Once conditions for the attachment of nanobodies to cell surfaces had been established, the ability of nanobody-cell conjugates to engage in targeted cell-cell interactions was explored. For this, a previously reported nanobody that binds the human epidermal growth factor receptor 2 (HER2) was expressed with the same C-terminal Ser-Gly₄-Tyr tag (nbHER2_{Tyr}). The upregulation of HER2 is a hallmark of many breast cancers and makes targeting this receptor relevant for therapeutic purposes. Many HER2 targeting drugs are already available in the clinic where they show great efficacy against HER2⁺ cancers. To validate that the conjugation of nbHER2_{T}, to proteins on the cell surface would not perturb HER2 binding, a nbHER2_{Tyr}-sfGFP conjugate was synthesized using a sfGFP Y²⁰⁰C mutant and abTYR. When exposed to the HER2⁺ cell line SK-BR-3, this construct produced a distinct shift in fluorescence indicative of successful binding. In comparison, the HER2-cell line MDA-MB-468 exhibited no change in fluorescence, indicating that the oquinoid linkage connecting the two proteins did not lead to off target binding.

Once again, 1×10⁶ NK-92MI cells were treated with 10 μM of nbHER2_{Tyr} in the presence or absence of 400 nM abTYR for 10 mins at 37° C. and with 5% CO₂. After washing, the cells were assayed for their ability to bind FITC-labeled HER2 in solution. Only cells treated with the combination of nbHER2_{Tyr} and abTYR were able to bind FITC-labeled HER2, as evidenced by a shift in the population of cells exhibiting fluorescence over an untreatment control (FIG. 3A, red trace). To represent an isotype control, no shift in fluorescence was observed for cells treated with nbGFP^{Tyr} and abTYR (FIG. 3A, blue trace). This indicates that antigen-binding of the conjugates is dependent on the target of the nanobody and is not influenced by the o-quinone-derived linkage between the nanobody and the cell surface.

FIG. 3A-3C. Decoration of NK cells for nanobody-directed cell-cell interactions. FIG. 3A. Using tyrosinase, a tyr-tagged nanobody against HER2 (nbHER2_{Tyr}) was attached to NK cells. Secondary labeling with a soluble FITC-HER2 showed that only cells exposed to nbHER2_{Tyr} and tyrosinase exhibited a shift in FITC signal detected via flow cytometry (red trace) over controls. FIG. 3B. To assess if tyrosinase-synthesized NK-nbHER2 conjugates can make targeted contacts with HER2⁺ cells, NK-nbHER2 cells were mixed with a HER2⁺ cell line (SK-BR-3) at a ratio of 2:1 (NK:target). Cells were allowed to bind and settle and then imaged using fluorescence microscopy. A nearest neighbor analysis was performed (CellProfiler), indicating that a statistically significant proportion of target cells (green) were bound to two or more NK-nbHER2 cells (red) only when the NK cells were pretreated with nbHER2_{Tyr} and tyrosinase (orange bar). FIG. 3C. Fluorescence microscopy images confirm rosette formation is only seen when NK cells are pretreated with both nHER2_{Tyr} and tyrosinase.

An important component of NK engagement is the binding of these cells to cell targets. To validate that tyrosinase-synthesized NK-nbHER2_{Tyr} could bind HER2⁺ cells, nanobody-NK conjugates were synthesized as described above. After synthesis, the conjugated NK cells were labeled with a MitoTracker Red dye. Simultaneously, the HER2⁺ cell line SK-BR-3 was labeled with a green CFSE dye, and the two cell types were mixed at a ratio of 2:1 (NK-conjugate:SK-BR-3). Cells were allowed to bind and settle for 2 h at 37° C., 5% CO₂, after which they were imaged using a high throughput fluorescence microscope (ImageXpress Micro, Molecular Devices). A nearest neighbor analysis was performed using CellProfiler (Broad Institute) to determine the number of red-labeled NK cells touching each green-labeled target SK-BR-3 cell. A greater proportion of green target cells were bound by two or more red NK cells only if they had been pre-treated with 10 μM nbHER2_{Tyr} and 400 nM abTYR for 10 mins (FIG. 3B, orange bar). Absence of abTYR or conjugation with nbGFP_{Tyrr} resulted in no statistically significant binding of target cells over untreated controls (FIG. 3B, blue bars). Finally, a rosette pattern of binding between the green HER2⁺ cells and the red NK cells, commonly observed when cells are forming cell:cell contacts, was only observed for NK cells treated with nbHER2_{T}yr and abTYR (FIG. 3C). Taken together, these results indicate that the tyrosinase-synthesized nanobody-NK conjugates form specific contacts with HER2⁺ cell types in a manner dependent on the antigen target of the nanobody.

Targeted Killing of nbHER2_{Tyr}-NK Cell Conjugates

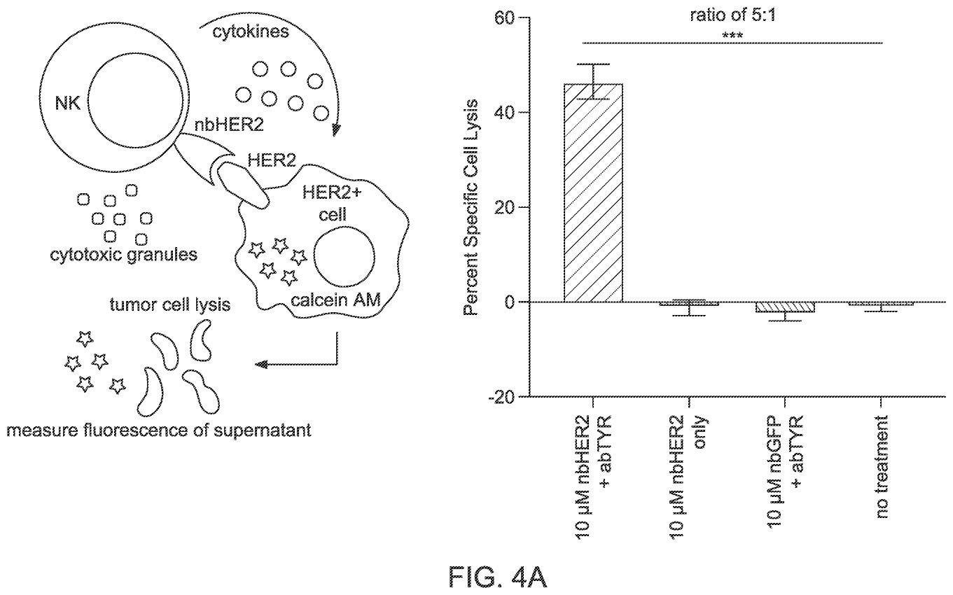

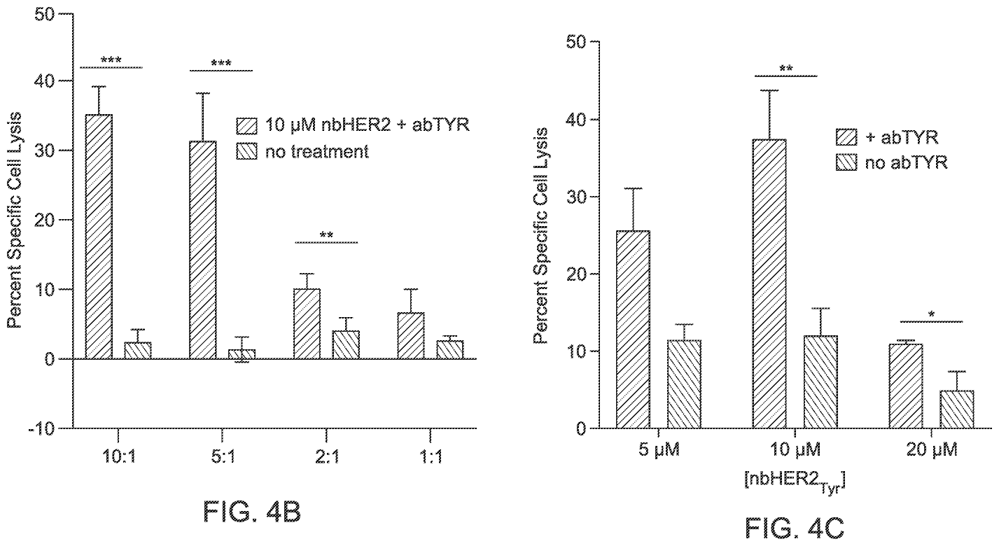

The ability of tyrosinase-synthesized nanobody-NK conjugates to perform their effector functions and elicit targeted cell death was explored. A fluorescence-based assay was used to measure NK-induced cell lysis. Briefly, HER2⁺ SK-BR-3 cells were preloaded with calcein AM dye, which becomes cell impermeable after uptake. NK-induced cell death permeabilizes the cell membrane, and leakage of the dye into the supernatant allows fluorescent determination of NK cytotoxicity (FIG. 4A).

NK-92MI cells were treated with nbHER2_{Tyr} in the presence or absence of abTYR. As an isotype control, NK cells were also treated with nbGFP_{Tyr} and abTYR. After treatment, cells were mixed with the calcein AM-labeled SK-BR-3 cells at a ratio of 5:1 (NK:SK-BR-3) and allowed to interact at 37° C., 5% CO₂. After 4 h, cells were spun down and the supernatant was harvested for fluorescence determination. A statistically significant cytotoxic response was only observed in the NK cells pretreated with both nbHER2_{Tyr} and abTYR (FIG. 4A, orange bar). Exposure of NK cells to nbHER2_{Tyr}r alone did not elicit any significant response over no treatment controls, indicating that the cytotoxic response requires direct conjugation between the nanobody and the cell surface (FIG. 4A). In addition, no significant cell death was observed in NK cells conjugated to nbGFP_{Tyr} via abTYR (FIG. 4A). This indicates that is not the o-quinone-derived linkage between the nanobody and the cell surface nor the exposure of the NK cells to abTYR itself that result in cell lysis. Upon varying the ratio of NK-nbHER2_{Tyr} conjugates to SK-BR-3 cells, it was found that the conjugates were capable of eliciting targeted cell death even at ratios as low as 2:1 (NK-conjugate:SK-BR-3, FIG. 4B).

FIG. 4A-4C. Targeted cell killing elicited by tyrosinase-synthesized nanobody-NK cell conjugates. FIG. 4A. Schematic representation of the fluorescence-based cell assay used to determine NK cytotoxicity. HER2⁺ cells (SK-BR-3) were preloaded with calcein AM dye, which is retained by the cell membrane after uptake. Lysis of HER2⁺ cells releases dye into the supernatant, providing a measurement for cell lysis. Only NK cells pretreated with both nbHER2_{Tyr}r and tyrosinase (orang bar) show statistically significant specific cell lysis over control treatments. FIG. 4B. To assess how the ratio of NK:target cell impacts specific cytotoxicity, NK-nbHER2 cells were synthesized using 10 μM nbHER2_{Tyr} and 400 nM tyrosinase and mixed with calcein AM loaded HER2⁺ cells (SK-BR-3). Statistically significant cell death was observed at ratios even as low as 2:1 (effector:target). FIG. 4C. To assess the required concentration of nbHER2_{Tyr} needed to elicit NK-mediated cell death, a variety of concentrations of nbHER2_{Tyr} were used to label NK cells with tyrosinase. Increased lysis was observed when using 5 μM and 10 μM nbHER2, while a sharp reduction of NK lytic activity was observed at the higher concentration of 20 μM nbHER2r.

The effect of different concentrations of nbHER2r, in the abTYR coupling step on the lysis ability of the resulting NK cell conjugates was examined. An increase in the ability of the NK-conjugates to elicit cell death was observed when they were exposed to nbHER2_{Tyr} concentrations up to 10 μM. At the highest nbHER_{Tyr} concentration of 20 μM, however, the sharp decrease in cell lysis ability suggests that there is an optimal density of binding proteins on the cell surfaces, beyond which the NK cells lose their ability to engage their effector functions (FIG. 4C). It is likely that very high levels of nanobody labeling preclude the binding of NK activating receptors to their cognate ligands.

One mechanism by which NK cells mediate their cytotoxic functionality is through NKG2D engagement of MICA/B on target cell surfaces. The role of the NKG2D-MICA/B axis in the cell death mediated by the synthesized NK-nbHER2_{Tyr} conjugates was explored. The same cell killing assays were performed as above, but blocked either NKG2D on the NK cell or MICA/B on the SK-BR-3 target cell using their respective antibodies. As expected, when MICA/B was blocked on the target cell, a statistically significant reduction in cell death was observed when compared to an isotype control. Blocking NKG2D had no effect compared to an isotype control. Modification of the NKG2D receptor by the conjugated nanobody may occlude the antibody epitope. However, the marked decrease in cell killing observed after blocking MICA/B indicates that the NKG2D receptor-ligand axis can still engage and plays an important role in mediating the observed cell killing.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A compound of
(i) formula (VI) or (VIA): 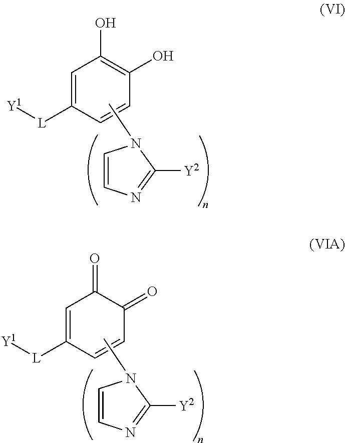
or a salt thereof, wherein:
Y¹ is a biomolecule, optionally comprising one or more moieties selected from, an active small molecule, an affinity tag, a fluorophore, and a metal-chelating agent;
L is an optional linker;
Y² is a second biomolecule; and
n is an integer from 1 to 3; or
(ii) formula (V) or (VA): 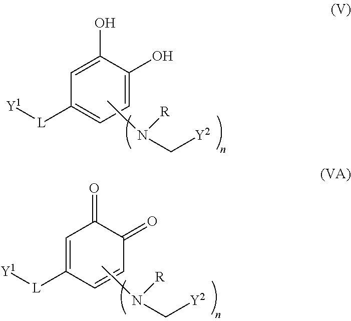
or a salt thereof, wherein:
Y¹ is a biomolecule, optionally comprising one or more moieties selected from, an active small molecule, an affinity tag, a fluorophore, and a metal-chelating agent;
L is an optional linker;
Y² is a second biomolecule;
R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
n is an integer from 1 to 3.

2.-10. (canceled)

11. The compound of claim 1, wherein:
(i) Y¹ and Y² are each a polypeptide;
(ii) Y¹ is selected from a fluorescent protein, an antibody, and an enzyme; and/or Y² is selected from a fluorescent protein, an antibody, and an enzyme;
(iii) Y¹ and/or Y² is an antibody; or
(iv) Y¹ and/or Y² is a cell.

12. The compound of claim 1, wherein Y¹ is selected from a fluorescent protein, an antibody, and an enzyme; and/or Y² is selected from a fluorescent protein, an antibody, and an enzyme.

13.-17. (canceled)

18. The compound of claim 12, wherein the antibody at Y¹ or Y² is selected from the group consisting of a single-domain antibody, an IgG1 isotype antibody or fragment thereof, an IgG2 isotype antibody or fragment thereof, an IgG3 isotype antibody or fragment thereof, an IgG4 isotype antibody or fragment thereof, an IgE isotype antibody or fragment thereof, an IgM isotype antibody or fragment thereof, and an Fc domain.

19.-20. (canceled)

21. The compound of claim 12, wherein the antibody at Y¹ or Y² is modified to (i) include a tyrosine residue within 5 amino acids of the C-terminus amino acid of the antibody or (ii) include a C-terminal tyrosine residue.

22.-32. (canceled)

33. The compound of claim 1, wherein n is 2 or 1.

34. (canceled)

35. A composition, comprising:
(i) a target molecule comprising an imidazole of formula (VII): 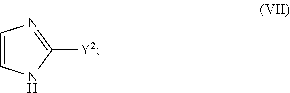
and
a biomolecule comprising a phenol moiety or a catechol moiety of formula (I): 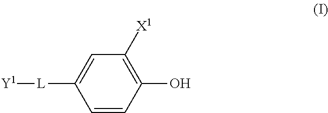
wherein:
Y¹ is a biomolecule, optionally comprising one or more moieties selected from, an active small molecule, an affinity tag, a fluorophore, and a metal-chelating agent;
X¹ is selected from hydrogen and hydroxyl;
L is an optional linker; and
Y² is a second biomolecule; or
(ii) a target molecule comprising an amine of formula (VIII): 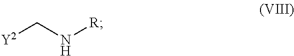
and
a biomolecule comprising a phenol moiety or a catechol moiety of formula (I): 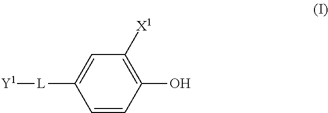
wherein:
Y¹ is a biomolecule, optionally comprising one or more moieties selected from, an active small molecule, an affinity tag, a fluorophore, and a metal-chelating agent;
X¹ is selected from hydrogen and hydroxyl;
L is an optional linker;
Y is a second biomolecule; and
R is selected from hydrogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

36. (canceled)

37. A compound comprising a first biomolecule attached to a second biomolecule, wherein the compound is produced by contacting a second biomolecule in vitro with a first biomolecule comprising a reactive moiety, wherein the second biomolecule comprises on its surface a surface molecule comprising a thiol, an amine, or an imidazole moiety; wherein the first biomolecule comprising the reactive moiety is generated by reaction of a first biomolecule comprising a phenol moiety or a catechol moiety with an enzyme capable of oxidizing the phenol or catechol moiety; and wherein said contacting is under conditions sufficient for conjugation of the surface molecule to the first biomolecule, thereby producing the compound, wherein the enzyme is a tyrosinase polypeptide.

38. The compound of claim 37, wherein the second biomolecule is a cell and comprises on its surface receptor a surface molecule comprising a thiol.

39. The compound of claim 37, wherein the first biomolecule and/or second biomolecule is a cell or an antibody.

40.-46. (canceled)

47. The compound of claim 37, wherein the antibody is modified to (i) include a tyrosine residue within 5 amino acids of the C-terminus amino acid of the antibody or (ii) include a C-terminal tyrosine residue.

48.-49. (canceled)

50. The compound of claim 37, wherein the tyrosinase polypeptide comprises (i) an amino acid sequence having at least 75% amino acid sequence identity to the abTYR amino acid sequence depicted in FIG. 9 or FIG. 10 or (ii) an amino acid sequence having at least 75% amino acid sequence identity to any one of the amino acid sequences depicted in any one of FIG. 11A-11Z and 11AA-11VV.

51. (canceled)

52. The compound of claim 37, wherein the phenol moiety is present in a tyrosine residue, the thiol moiety is present in a cysteine residue, the amine moiety is present in a lysine residue, or the imidazole moiety is present in a histidine residue.

53.-56. (canceled)

57. The compound of claim 37, wherein the reactive moiety is an orthoquinone or a semi-quinone radical, or a combination thereof.

58. The compound of claim 37, wherein the cell is an immune cell.

59.-65. (canceled)

66. A method for attachment of a first biomolecule to a second biomolecule, the method comprising:
contacting a second biomolecule in vitro with a first biomolecule comprising a reactive moiety, wherein the second biomolecule comprises on its surface a surface molecule comprising a thiol, an amine, or an imidazole moiety;
wherein the first biomolecule comprising the reactive moiety is generated by reaction of a first biomolecule comprising a phenol moiety or a catechol moiety with an enzyme capable of oxidizing the phenol or catechol moiety; and wherein said contacting is under conditions sufficient for conjugation of the surface molecule to the first biomolecule, thereby producing a modified biomolecule, wherein the enzyme is a tyrosinase polypeptide.

67. The method of claim 66, wherein the second biomolecule is a cell and comprises on its surface receptor a surface molecule comprising a thiol.

68. The method of claim 66, wherein the first biomolecule and/or second biomolecule is a cell or an antibody.

69.-80. (canceled)

81. The method of claim 66, wherein the phenol moiety is present in a tyrosine residue, the thiol moiety is present in a cysteine residue, the amine moiety is present in a lysine residue, or the imidazole moiety is present in a histidine residue.

82.-99. (canceled)


SEQUENCE LISTING (208 sequences)

SEQ ID NO: 1 - protein, 382 aa, Agricus bisporus
SLLATVGPTGGVKNRLDIVDFVRDEKFFTLYIRALQAIQDKDQSDYSSFFQLSGIHGLPFTPWAKPKDTPTVPYESGYCTHSQVLFPTWHRVYVSIYEQILQEAAKGIAKKFTVHKKEWAQAAEDLRQPYWDTGFALVPPDEIIKLEQVKITNYDGTKITVRNPILRYSFHPIDPSFNGYPNFDTWKTTVRNPDADKKENIPALIGKLDLEADSTREKTYNMLKFNANWEAFSNHGEFDDTHANSLEAVHDDIHGFVGRGAIRGHMTHALFAAFDPIFWLHHSNVDRHLSLWQALYPGVWVTQGPEREGSMGFAPGTELNKDSALEPFYETEDKPWTSVPLTDTALLNYSYPDFDKVKGGTPDLVRDYINDHIDRRYGIKKS

SEQ ID NO: 2 - protein, 391 aa, Agricus bisporus
SDKKSLMPLVGIPGEIKNRLNILDFVKNDKFFTLYVRALQVLQARDQSDYSSFFQLGGIHGLPYTEWAKAQPQLHLYKANYCTHGTVLFPTWHRAYESTWEQTLWEAAGTVAQRFTTSDQAEWIQAAKDLRQPFWDWGYWPNDPDFIGLPDQVIRDKQVEITDYNGTKIEVENPILHYKFHPIEPTFEGDFAQWQTTMRYPDVQKQENIEGMIAGIKAAAPGFREWTFNMLTKNYTWELFSNHGAVVGAHANSLEMVHNTVHFLIGRDPTLDPLVPGHMGSVPHAAFDPIFWMHHCNVDRLLALWQTMNYDVYVSEGMNREATMGLIPGQVLTEDSPLEPFYTKNQDPWQSDDLEDWETLGFSYPDFDPVKGKSKEEKSVYINDWVHKHYG

SEQ ID NO: 3 - protein, 305 aa, Bacillus megaterium
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 4 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 5 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 6 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 7 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 8 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 9 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 10 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 11 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 12 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 13 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 14 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 15 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 16 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 17 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 18 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 19 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 20 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 21 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 22 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 23 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 24 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGFINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 25 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 26 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDEQGNPSGGLKRNFGATKEAPTLPTRDDVLNALKITQYDTPPWDMTSQNSFRNQLEGAINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 27 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 28 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 29 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 30 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 31 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 32 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 33 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 34 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 35 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 36 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 37 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 38 - protein, 305 aa, synthetic construct
MSNKYRVRKNVLHLTDTEKRDFVRTVLILKEKGIYDRYIAWHGAAGKFHTPPGSDRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 39 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 40 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHRWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 41 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 42 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 43 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 44 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHHWVGGQMGVVPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 45 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHRWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 46 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHRWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 47 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 48 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGFINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 49 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHHWVGGQMGVGPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 50 - protein, 305 aa, synthetic construct
MSNKYRVRKNVLHLTDTEKRDFVRTVLILKEKGIYDRYIAWHGAAGKFHTPPGSKRNAAHMSSAFLPWHREYLLRFERDLQSINPEVTLPYWEWETDAQMQDPSQSQIWSADFMGGNGNPIKDFIVDTGPFAAGRWTTIDKQGNPSGGLKRNFGATKEAPTLPTRDDVLNALKITQYDTPPWDMTSQNSFRNQLEGAINGPQLHNRVHHWVGGQMGVFPTAPNDPVFFLHHANVDRIWAVWQIIHRNQNYQPMKNGPFGQNFRDPMYPWNTTPEDVMNHRKLGYVYDIELRKSKRSSLEHHHHHH

SEQ ID NO: 51-52 - skipped (2 entries)

SEQ ID NO: 53 - protein, 6 aa, synthetic construct
  VARIANT 2: note = Wherein X is L or Q
PXGMTS

SEQ ID NO: 54 - protein, 5 aa, synthetic construct
  VARIANT 2: note = Wherein X is L or Q
PXGMT

SEQ ID NO: 55 - protein, 7 aa, synthetic construct
ENLYTQS

SEQ ID NO: 56 - protein, 7 aa, synthetic construct
ENLYFQG

SEQ ID NO: 57 - protein, 9 aa, synthetic construct
ENLYFQGGY

SEQ ID NO: 58 - protein, 7 aa, synthetic construct
ENLYFQS

SEQ ID NO: 59 - protein, 5 aa, synthetic construct
DDDDK

SEQ ID NO: 60 - protein, 4 aa, synthetic construct
LVPR

SEQ ID NO: 61 - protein, 8 aa, synthetic construct
LEVLFQGP

SEQ ID NO: 62 - protein, 10 aa, synthetic construct
CGLVPAGSGP

SEQ ID NO: 63 - protein, 12 aa, synthetic construct
SLLKSRMVPNFN

SEQ ID NO: 64 - protein, 12 aa, synthetic construct
SLLIARRMPNFN

SEQ ID NO: 65 - protein, 12 aa, synthetic construct
SKLVQASASGVN

SEQ ID NO: 66 - protein, 12 aa, synthetic construct
SSYLKASDAPDN

SEQ ID NO: 67 - protein, 12 aa, synthetic construct
RPKPQQFFGLMN

SEQ ID NO: 68 - protein, 12 aa, synthetic construct
SLRPLALWRSFN

SEQ ID NO: 69 - protein, 12 aa, synthetic construct
SPQGIAGQRNFN

SEQ ID NO: 70 - protein, 14 aa, synthetic construct
DVDERDVRGFASFL

SEQ ID NO: 71 - protein, 12 aa, synthetic construct
SLPLGLWAPNFN

SEQ ID NO: 72 - protein, 12 aa, synthetic construct
SLLIFRSWANFN

SEQ ID NO: 73 - protein, 12 aa, synthetic construct
SGVVIATVIVIT

SEQ ID NO: 74 - protein, 12 aa, synthetic construct
SLGPQGIWGQFN

SEQ ID NO: 75 - protein, 12 aa, synthetic construct
KKSPGRVVGGSV

SEQ ID NO: 76 - protein, 12 aa, synthetic construct
PQGLLGAPGILG

SEQ ID NO: 77 - protein, 31 aa, synthetic construct
HGPEGLRVGFYESDVMGRGHARLVHVEEPHT

SEQ ID NO: 78 - protein, 12 aa, synthetic construct
GPQGLAGQRGIV

SEQ ID NO: 79 - protein, 12 aa, synthetic construct
GGSGQRGRKALE

SEQ ID NO: 80 - protein, 12 aa, synthetic construct
SLSALLSSDIFN

SEQ ID NO: 81 - protein, 12 aa, synthetic construct
SLPRFKIIGGFN

SEQ ID NO: 82 - protein, 12 aa, synthetic construct
SLLGIAVPGNFN

SEQ ID NO: 83 - protein, 12 aa, synthetic construct
FFKNIVTPRTPP

SEQ ID NO: 84 - protein, 20 aa, synthetic construct
  VARIANT 1..20: note = Wherein up to 4 copies of GGGG may be deleted
GGGGGGGGGGGGGGGGGGGG

SEQ ID NO: 85 - protein, 5 aa, synthetic construct
SGGGG

SEQ ID NO: 86 - protein, 25 aa, synthetic construct
  VARIANT 1..25: note = Wherein up to 4 copies of GGGGS may be deleted
GGGGSGGGGSGGGGSGGGGSGGGGS

SEQ ID NO: 87 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 107 aa, synthetic construct [sequence omitted]

SEQ ID NO: 90 - protein, 120 aa, synthetic construct [sequence omitted]

SEQ ID NO: 91 - protein, 11 aa, synthetic construct
RASQDVNTAVA

SEQ ID NO: 92 - protein, 6 aa, synthetic construct
SASFLY

SEQ ID NO: 93 - protein, 8 aa, synthetic construct
QQHYTTPP

SEQ ID NO: 94 - protein, 8 aa, synthetic construct
GFNIKDTY

SEQ ID NO: 95 - protein, 8 aa, synthetic construct
IYPTNGYT

SEQ ID NO: 96 - protein, 13 aa, synthetic construct
SRWGGDGFYAMDY

SEQ ID NO: 97 - protein, 242 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 98 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 107 aa, synthetic construct [sequence omitted]

SEQ ID NO: 101 - protein, 119 aa, synthetic construct [sequence omitted]

SEQ ID NO: 102 - protein, 11 aa, synthetic construct
KASQDVSIGVA

SEQ ID NO: 103 - protein, 6 aa, synthetic construct
SASYRY

SEQ ID NO: 104 - protein, 8 aa, synthetic construct
QQYYIYPY

SEQ ID NO: 105 - protein, 10 aa, synthetic construct
GFTFTDYTMD

SEQ ID NO: 106 - protein, 18 aa, synthetic construct
ADVNPNSGGSIYNQRFKG

SEQ ID NO: 107 - protein, 12 aa, synthetic construct
ARNLGPSFYFDY

SEQ ID NO: 108 - protein, 134 aa, synthetic construct
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSAAAHHHHHHSGGGGY

SEQ ID NO: 109 - protein, 128 aa, synthetic construct
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSAAASGGGGY

SEQ ID NO: 110 - protein, 127 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSAAASGGGGY

SEQ ID NO: 111 - protein, 124 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSSGGGGY

SEQ ID NO: 112 - protein, 118 aa, synthetic construct
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 113 - protein, 15 aa, synthetic construct
KASQSVDYDGDSYLN

SEQ ID NO: 114 - protein, 7 aa, synthetic construct
DASNLVS

SEQ ID NO: 115 - protein, 9 aa, synthetic construct
QQSTEDPWT

SEQ ID NO: 116 - protein, 5 aa, synthetic construct
SYWMN

SEQ ID NO: 117 - protein, 17 aa, synthetic construct
QIWPGDGDTNYNGKFKG

SEQ ID NO: 118 - protein, 15 aa, synthetic construct
RETTTVGRYYYAMDY

SEQ ID NO: 119 - protein, 249 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVS

SEQ ID NO: 120 - protein, 217 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTESS

SEQ ID NO: 121 - protein, 450 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 107 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTK

SEQ ID NO: 123 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 124 - protein, 14 aa, synthetic construct
TGTSSDIGGYNSVS

SEQ ID NO: 125 - protein, 11 aa, synthetic construct
LMIYGVNNRPS

SEQ ID NO: 126 - protein, 10 aa, synthetic construct
SSYDIESATP

SEQ ID NO: 127 - protein, 10 aa, synthetic construct
GYSFTSYWIG

SEQ ID NO: 128 - protein, 16 aa, synthetic construct
WMGIIDPGDSRTRYSP

SEQ ID NO: 129 - protein, 11 aa, synthetic construct
GQLYGGTYMDG

SEQ ID NO: 130 - protein, 244 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 131 - protein, 253 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 132 - protein, 217 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPDSKQSNNKYAASSYLSLTPEQWKSHRSYSCQVTHEGSTVEKTVAPTECS

SEQ ID NO: 133 - protein, 453 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 135 - protein, 131 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSV

SEQ ID NO: 136 - protein, 8 aa, synthetic construct
SSNIGSNT

SEQ ID NO: 137 - protein, 11 aa, synthetic construct
AAWDDSLNGWV

SEQ ID NO: 138 - protein, 8 aa, synthetic construct
GFTFGDYA

SEQ ID NO: 139 - protein, 10 aa, synthetic construct
SRSKAYGGTT

SEQ ID NO: 140 - protein, 14 aa, synthetic construct
ASSGYSSGWTPFDY

SEQ ID NO: 141 - protein, 249 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 142 - protein, 249 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 143 - protein, 11 aa, synthetic construct
SASQDISNYLN

SEQ ID NO: 144 - protein, 7 aa, synthetic construct
YTSNLHS

SEQ ID NO: 145 - protein, 9 aa, synthetic construct
QQYRKLPWT

SEQ ID NO: 146 - protein, 5 aa, synthetic construct
NYWMH

SEQ ID NO: 147 - protein, 17 aa, synthetic construct
ATYRGHSDTYYNQKFKG

SEQ ID NO: 148 - protein, 12 aa, synthetic construct
GAIYNGYDVLDN

SEQ ID NO: 149 - protein, 108 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 150 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 151 - protein, 43 aa, synthetic construct
SNIKFRPGSVVVQLTLAFREGTINVHDVETQFNQYKTEAASRY

SEQ ID NO: 152 - protein, 35 aa, synthetic construct
SVVVQLTLAFREGTINVHDVETQFNQYKTEAASRY

SEQ ID NO: 153 - protein, 20 aa, synthetic construct
LAFREGTINVHDVETQFNQY

SEQ ID NO: 154 - protein, 25 aa, synthetic construct
SNIKFRPGSVVVQLTLAAFREGTIN

SEQ ID NO: 155 - protein, 5 aa, synthetic construct
RYGMS

SEQ ID NO: 156 - protein, 17 aa, synthetic construct
TISGGGTYIYYPDSVKG

SEQ ID NO: 157 - protein, 13 aa, synthetic construct
DNYGRNYDYGMDY

SEQ ID NO: 158 - protein, 10 aa, synthetic construct
SATSSVSYIH

SEQ ID NO: 159 - protein, 7 aa, synthetic construct
STSNLAS

SEQ ID NO: 160 - protein, 9 aa, synthetic construct
QQRSSSPFT

SEQ ID NO: 161 - protein, 5 aa, synthetic construct
GYAMS

SEQ ID NO: 162 - protein, 17 aa, synthetic construct
TISSGGTYIYYPDSVKG

SEQ ID NO: 163 - protein, 12 aa, synthetic construct
LGGDNYYEYFDV

SEQ ID NO: 164 - protein, 15 aa, synthetic construct
RASKSVSTSGYSYMH

SEQ ID NO: 165 - protein, 7 aa, synthetic construct
LASNLES

SEQ ID NO: 166 - protein, 9 aa, synthetic construct
QHSRELPFT

SEQ ID NO: 167 - protein, 5 aa, synthetic construct
DYAMN

SEQ ID NO: 168 - protein, 17 aa, synthetic construct
VISTFSGNINFNQKFKG

SEQ ID NO: 169 - protein, 10 aa, synthetic construct
SDYYGPYFDY

SEQ ID NO: 170 - protein, 16 aa, synthetic construct
RSSQTIVHSNGNTYLE

SEQ ID NO: 171 - protein, 7 aa, synthetic construct
KVSNRFS

SEQ ID NO: 172 - protein, 9 aa, synthetic construct
FQGSHVPFT

SEQ ID NO: 173 - protein, 9 aa, synthetic construct
LGGDNYYEY

SEQ ID NO: 174 - protein, 15 aa, synthetic construct
TASKSVSTSGYSYMH

SEQ ID NO: 175 - protein, 7 aa, synthetic construct
LVSNLES

SEQ ID NO: 176 - protein, 10 aa, synthetic construct
QHIRELTRSE

SEQ ID NO: 177 - protein, 8 aa, synthetic construct
GFTFSNYY

SEQ ID NO: 178 - protein, 8 aa, synthetic construct
ISGRGSTI

SEQ ID NO: 179 - protein, 10 aa, synthetic construct
VKDRGGYSPY

SEQ ID NO: 180 - protein, 6 aa, synthetic construct
QSISTY

SEQ ID NO: 181 - protein, 10 aa, synthetic construct
QQSYSTPPIT

SEQ ID NO: 182 - protein, 15 aa, synthetic construct
TEDEVQSYPSKHDYV

SEQ ID NO: 183 - protein, 12 aa, synthetic construct
EVQSYPSKHDYV

SEQ ID NO: 184 - protein, 8 aa, synthetic construct
GYTFTDYS

SEQ ID NO: 185 - protein, 8 aa, synthetic construct
INTETGVP

SEQ ID NO: 186 - protein, 8 aa, synthetic construct
ARRTGFDY

SEQ ID NO: 187 - protein, 11 aa, synthetic construct
KNLLHSDGITY

SEQ ID NO: 188 - protein, 9 aa, synthetic construct
VQVLELPFT

SEQ ID NO: 189 - protein, 8 aa, synthetic construct
GFTFSSYA

SEQ ID NO: 190 - protein, 8 aa, synthetic construct
ISDGGSYS

SEQ ID NO: 191 - protein, 14 aa, synthetic construct
ARDSYYDNSYVRDY

SEQ ID NO: 192 - protein, 6 aa, synthetic construct
QDINTF

SEQ ID NO: 193 - protein, 9 aa, synthetic construct
LQYDEFPLT

SEQ ID NO: 194 - DNA, 488 bp, synthetic construct
  source 1..488: mol_type = other DNA
tgagcggataacaattcccctctagaaataattttgtttaactttaagaaggagatataccatggcagatgttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcgcagcagcacatcatcaccatcatcattcaggtggtggtggttattgactcgagcaccaccaccaccaccactgag

SEQ ID NO: 195 - protein, 132 aa, synthetic construct
MADVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSAAAHHHHHHSGGGGY

SEQ ID NO: 196 - protein, 6 aa, synthetic construct
SGGGGY

SEQ ID NO: 197 - DNA, 494 bp, synthetic construct
  source 1..494: mol_type = other DNA
tgagcggataacaattcccctctagaaataattttgtttaactttaagaaggagatataccatggaagttcagctggttgaaagcggtggtggtctggttcaggcaggcggtagcctgcgtctgagctgtgcagcaagcggtattacctttagcattaataccatgggttggtatcgtcaggcaccgggtaaacagcgtgaactggttgcactgattagcagcattggtgatacctattatgccgatagcgttaaaggtcgttttaccattagccgtgataatgccaaaaataccgtttacctgcagatgaatagcctgaaaccggaagataccgcagtgtattattgtaaacgttttcgtaccgcagcacagggcaccgattattggggtcagggcacccaggttaccgttagcagcgcagcagcacatcatcaccatcatcattcaggtggtggtggttattgactcgagcaccaccaccaccaccactgag

SEQ ID NO: 198 - DNA, 18 bp, synthetic construct
  source 1..18: mol_type = other DNA
ctcgatcccgcgaaatta

SEQ ID NO: 199 - protein, 20 aa, synthetic construct
VTSAPDTRPAPGSTAPPAHG

SEQ ID NO: 200 - protein, 5 aa, synthetic construct
GGGGS

SEQ ID NO: 201 - DNA, 38 bp, synthetic construct
  source 1..38: mol_type = other DNA
atcattcaggtggtggtggttgttgactcgagcaccac

SEQ ID NO: 202 - DNA, 38 bp, synthetic construct
  source 1..38: mol_type = other DNA
gtggtgctcgagtcaacaaccaccaccacctgaatgat

SEQ ID NO: 203 - DNA, 47 bp, synthetic construct
  source 1..47: mol_type = other DNA
gcagataaacggtattacgacaatcatcacggctgatggtaaaacga

SEQ ID NO: 204 - DNA, 47 bp, synthetic construct
  source 1..47: mol_type = other DNA
tcgttttaccatcagccgtgatgattgtcgtaataccgtttatctgc

SEQ ID NO: 205 - DNA, 396 bp, synthetic construct
  source 1..396: mol_type = other DNA
atggcagatgttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcgcagcagcacatcatcaccatcatcattcaggtggtggtggttgt

SEQ ID NO: 206 - protein, 132 aa, synthetic construct
MADVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSAAAHHHHHHSGGGGC

SEQ ID NO: 207 - DNA, 396 bp, synthetic construct
  source 1..396: mol_type = other DNA
atggcagatgttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgattgtcgtaataccgtttatctgcaaatgaatagcctgaaaccggaagataccgcagtgtattattgcaatgttaacgtgggctttgaatattggggtcagggcacccaggttaccgttagcagcgcagcagcacatcatcaccatcatcattcaggtggtggtggttat

SEQ ID NO: 208 - protein, 132 aa, synthetic construct
MADVQLVESGGALVQPGGSLRLSCAASGFPVNRYSMRWYRQAPGKEREWVAGMSSAGDRSSYEDSVKGRFTISRDDCRNTVYLQMNSLKPEDTAVYYCNVNVGFEYWGQGTQVTVSSAAAHHHHHHSGGGGY